(19) United States
(12) Patent Application Publication
(10) Pub. No.: US 2014/0088175 A1
(43) Pub. Date: Mar. 27, 2014

(54) TRANSDUCTION OF CELLS BY ADENO-ASSOCIATED VIRUS VECTORS

(71) Applicants: John A. Chiorini, Dayton, MD (US); Giovanni Di Pasquale, Kensington, MD (US)

(72) Inventors: John A. Chiorini, Dayton, MD (US); Giovanni Di Pasquale, Kensington, MD (US)

(73) Assignee: The USA, as represented by the Secretary, Dept. of Health and Human Services, Bethesda, MD (US)

(21) Appl. No.: 13/416,717

(22) Filed: Mar. 9, 2012

Related U.S. Application Data
(63) Continuation of application No. 11/574,954, filed on Mar. 8, 2007, now abandoned.
(60) Provisional application No. 60/607,854, filed on Sep. 8, 2004.

(51) Int. Cl. C12N 15/86

(52) U.S. Cl. CPC C12N 15/86
USPC 514/44 R; 435/456

(57) ABSTRACT

The present invention provides methods of transcytosing barrier epithelial cells using adeno-associated virus-4 (AAV4), adeno-associated virus-5 (AAV5), adeno-associated virus-7 (AAV7), bovine adeno-associated virus (BAAV), and vectors and particles derived therefrom. In addition, the present invention provides methods of delivering a nucleic acid across the barrier epithelia using the AAV4, AAV5, AAV7, and BAAV vectors and particles.

### CROSS-REFERENCE TO RELATED APPLICATIONS

This claims the benefit of U.S. Provisional Application No. 60/607,854, entitled “Transcytosis of Adeno-Associated Viruses”, filed Sep. 8, 2004, by Chiorini et al, which is herein incorporated by reference in its entirety.

### BACKGROUND

The adeno-associated viruses (AAV) were originally classified according to size, structure, and dependence upon a helper virus for replication. AAV is a member of the Parvoviridae, a virus family characterized by a single stranded linear DNA genome and a small icosahedral shaped capsid measuring about 20 nm in diameter. AAV was first described as a contaminant of tissue culture grown simian virus 15, a simian adeno virus and was found dependent on adenovirus for measurable replication. This led to its name, adeno-associated virus, and its classification in the genus Dependovirus. Because the majority of AAV isolates were first identified as contaminants of laboratory stocks of adenovirus, little is known about their natural tissue tropism. However in vivo experiments suggest they are effective vectors for gene transfer applications. Currently eleven full-length isolates have been cloned and their initial characterization indicates that each serotype has unique binding/cell tropism characteristics.

Transcytosis is the transport of macromolecular cargo from one side of a cell to the other within membrane-bounded carrier(s). It is a strategy used by multicellular organisms to selectively move material between two different environments while maintaining the distinct compositions of those environments. The ability of a pathogen to spread through a tissue is a critical determinate of its virulence. The process of transcytosis has been reported for a number of viruses. For example, HIV and poliovirus cross simple epithelial cells without infection and are still infectious when they cross into the submucosa. Likewise, the Epstein-Barr virus (EBV) forms a complex with mucosal immunoglobulins (IgA) that are specific for gp350, a viral surface protein that is present in latently infected people. This complex binds to the poly-immunoglobulin receptor at the basal surface of epithelial cells, and is endocytosed and delivered apically without infection. To date, there is no report of transcytosis by any AAV.

Provided herein are methods for transcytosis across barrier epithelial cells using AAV vectors. The ability of a non-pathogenic vector to transcytose barrier epithelial cells can be used to deliver genes to sub-epithelial targets. One important example includes the delivery of genes across the blood-brain-barrier without the need for direct injection into the brain. Furthermore, herein is described a method for re-directing virus that enters a cell by transcytosis to result in transduction of the cell by blocking exocytosis.

### SUMMARY

In accordance with the purpose(s) of this invention, as embodied and broadly described herein, this invention, in one aspect, relates to a method of delivering a heterologous nucleic acid across an epithelial barrier comprising delivering to the epithelial barrier an AAV vector comprising the heterologous nucleic acid. The epithelial cells can be in the gut, lung, genitourinary tract, kidney, blood vessels or brain.

In another aspect, the invention relates to a method of transcytosing epithelial cells of a human subject comprising administering to the subject a viral vector comprising a heterologous nucleic acid, wherein the viral vector is selected from a group consisting of BAAV, AAV4, AAV5, or AAV7.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate (one) several embodiment(s) of the invention and together with the description, serve to explain the principles of the invention.

FIG. 1 shows that AAV4 transcytosed in CaCo-2, MDCKI, MDCKII, Human primary immortalized epithelial endometrial, Bovine brain primary endothelia cells (BBB). AAV5 transcytosed CaCo-2 cells, whereas BAAV transcytosed in MDCKs, Endometrial, airways epithelia, and BBB. AAV6 did not transcytose in any of cell types tested. Hela cells do not form barrier epithelia and were used as a control.

FIG. 2 shows that the treatment of the basal lateral surface of Human primary airways epithelial cell (RAE) with tannic acid blocked the transcytosis of BAAV vector containing a GFP expression cassette from the apical surface to the basal lateral. Furthermore transduction dramatically increased when assayed at 24 hrs post inoculation. In contrast no change was observed in AAV2 transduction, which did not demonstrate any transcytosis activity and has limited binding activity on HAE.

FIG. 3 shows AAV7 transcytosis assay on bovine brain endothelial cells. Virus DNA extracted from basal lateral medium after 311 incubation 2×10⁹ DRP of AAV were loaded on the apical side of the cell layer. AAV5 is used as a control.

### DETAILED DESCRIPTION

The present invention may be understood more readily by reference to the following detailed description of the invention and the Examples included therein and to the Figures and their previous and following description.

Before the present compounds, compositions, articles, devices, and/or methods are disclosed and described, it is to be understood that this invention is not limited to specific synthetic methods, specific cell types, or to particular tissues, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” as used herein means that the subsequently described event or circumstance may or may not occur, and that the description includes instances where said event or circumstance occurs and instances where it does not.

### AAV Transcytosis

Disclosed is a method of delivering a heterologous nucleic acid across an epithelial barrier comprising delivering to the epithelial barrier an AAV vector comprising the heterologous nucleic acid. In one aspect of the method, the AAV is AAV4, AAV5, AAV7, or BAAV. The AAV capsid protein forming the viral particle is understood herein to confer upon the AAV particle the desired transcytosing ability. Thus, “AAV vector”, as used herein, refers to any virion, vector, or viral particle comprising or encoding at least one AAV capsid protein. As an example, an AAV4 vector can encode an AAV4 capsid protein and thus be encapsidated in said protein forming an AAV4 particle. Alternatively the AAV vector can comprise a nucleic acid encoding a modified AAV or a portion of an AAV capsid protein (a capsid protein fragment) that confers serotype-specific trancytotic activity. AAV capsids, capsid protein fragments and capsid modifications are disclosed, for example, in U.S. Patent Application No. 60/526786 (BAAV), U.S. Pat. No. 6,468,524 (AAV4), U.S. patent application Ser. No. 09/717,789 (AAV5), U.S. Patent Application 2003/0228282 (AAV7), International Application No. PCT/US04/15534, filed May 19, 2004 (AAAV), and U.S. Patent Application No. 60/676604, filed Apr. 29, 2005 (AAV-X1, AAV-X1b, AAV-X5, AAV-X19, AAV-X21, AAV-X22, AAV-X23, AAV-X24, AAV-X25, AAV-X26).

In another aspect of the method, the epithelial cells are in the gut, lung, genitourinary tract, kidney, blood vessels or brain. In another aspect of the method, the epithelial cells can be selected from a group consisting of bronchial, alveolar, tracheal or upper airway epithelial cells; absorptive enterocytes or M cells; endometrial or urinary epithelial cells; renal collecting duct or proximal tubule epithelial cells; cerebral microvascular endothelial cells or Choroidal Plexus epithelial cells.

Further disclosed is a method of transcytosing epithelial cells of a human subject comprising administering to the subject an AAV vector comprising a heterologous nucleic acid. In one aspect of the method, the vector is AAV4, AAV5, AAV7, or BAAV. In another aspect of the method, the epithelial cells are selected from a group consisting of bronchial, alveolar, tracheal or upper airway epithelial cells; absorptive enterocytes or M cells; endometrial or urinary epithelial cells; renal collecting duct or proximal tubule epithelial cells; cerebral microvascular endothelial cells or Choroidal Plexus epithelial cells.

Further contemplated are methods for the delivery of molecules across epithelial cell barriers comprising coupling the molecules to non-recombinant (wild-type) AAV capsids or particles. In one aspect, the molecules are radioligands or enzymes.

The term “adeno-associated virus (AAV)” is used herein to refer to a genus of viruses in the family Parvoviridae which are all defective viruses (unable to replicate by themselves) and depend on the co-infection of their host cell by other, nondefective viruses to help them replicate.

Transcytosis refers to the transport of macromolecular cargo from one side of a cell to the other, generally within a membrane-bounded carrier(s). Tuma and Hubbard provided a review of transcytosis (Tuma P L and Hubbard A L. 2003. Physiol Rev. 83:871-932), herein incorporated by reference for its teaching regarding the nature and uses for trancytosis. Transcytosis is a strategy used by multicellular organisms to selectively move material between two different environments while maintaining the distinct compositions of those environments. N. Simionescu was the first to coin the term transcytosis to describe the vectorial transfer of macromolecular cargo within the plasmalemmal vesicles from the circulation across capillary endothelial cells to the interstitium of tissues. During this same period, another type of transcytosis was being discovered. Immunologists comparing the different types of immunoglobulins found in various secretions (e.g., serum, milk, saliva, and the intestinal lumen) speculated that the form of IgA found in external secretions (called secretory IgA, due to the presence of an additional protein component) was selectively transported across the epithelial cell barrier. More is known about transcytosis as it is expressed in epithelial tissues, which form cellular barriers between two environments. In this polarized cell type, net movement of material can be in either direction, apical to basolateral or the reverse, depending on the cargo and particular cellular context of the process. However, transcytosis is not restricted to only epithelial cells.

Since the 19th century dye experiments of Ehrlich, the brain has been known as a “privileged” organ where access is tightly regulated so that the environment remains chemically stable. The two principal gatekeepers of the brain are the cerebral capillary endothelium and the cuboidal epithelial cells of the choroid plexus. These cellular barriers are specialized for the passage of different nutrients from the blood. The capillaries move nutrients that are required rapidly and in large quantities, such as glucose and amino acids. These small molecules are transported by membrane carriers using facilitated diffusion. The choroid plexus supplies nutrients that are required less acutely and in lower quantities. These are folate and other vitamins, ascorbate, and deoxyribonucleotides.

There are two epithelial cells that participate in transcytosis in the intestine, M cells and enterocytes (adsorptive columnar cells). These cells are very different from one another and the capillary endothelial cell. Depending on the species, M cells comprise a variable but small percentage of the epithelia overlying organized mucosal-associated lymphoid tissue, making them a very minor cell population in the gastrointestinal tract. The transcytotic route across M cells is thought to be part of the mechanism by which antigens are routinely sampled along the entire mucosal surface. Not surprisingly, numerous pathogens have evolved mechanisms to exploit the transcytotic process as a means to invade and disseminate before a strong enough immune response can be mounted.

Absorptive enterocytes are simple columnar cells with several apical features in addition to their brush borders. Clathrin-coated pits are present at the base of microvilli, and a thick glycocalyx composed of integral membrane proteins with glycosaminoglycan side chains emanates from the microvillar membrane. This latter structural feature as well as the rigidity of the microvilli are thought to prohibit microorganisms from attaching and invading enterocytes. The intracellular organization of these columnar epithelial cells is also polarized, with basally located nuclei, supranuclear Golgi, and an abundance of pleiomorphic membrane compartments underlying the terminal web of the brush border. The basolateral-to-apical length of this cell is ˜20 versus 0.2 μm for a capillary endothelial cell, making the transcytotic route across enterocytes potentially much longer. Furthermore, microtubules are an important structural element of the transcytotic pathway in enterocytes, but not in M or endothelial cells.

Transcytosis also occurs in the upper regions of the respiratory tract and has been demonstrated with two vector systems, pIgA-R and FcRn, but others could exist. Secretory IgA is a known constituent of the lung's immune defense system, with bronchial epithelial cells carrying out basolateral-to-apical transport of dIgA, which is secreted by local plasma cells in underlying lymphoid tissue. Albumin, which is found in lung fluid, is endocytosed specifically at the apical surface of airway epithelia but is then subsequently degraded. At the alveolar level, the question of whether albumin is transcytosed intact is uncertain.

The methods and compositions described herein can be used to deliver heterologous nucleic acids to certain tissues. As used herein, the term “nucleic acid” refers to single- or multiple stranded molecules which may be DNA or RNA, or any combination thereof, including modifications to those nucleic acids. The nucleic acid may represent a coding strand or its complement, or any combination thereof. Nucleic acids may be identical in sequence to the sequences which are naturally occurring for any of the novel genes discussed herein or may include alternative codons which encode the same amino acid as those provided herein, including that which is found in the naturally occurring, sequence. These nucleic acids can also be modified from their typical structure. Such modifications include, but are not limited to, methylated nucleic acids, the substitution of a non-bridging oxygen on the phosphate residue with either a sulfur (yielding phosphorothioate deoxynucleotides), selenium (yielding phosphorselenoate deoxynucleotides), or methyl groups (yielding methylphosphonate deoxynucleotides).

As used herein, the term “isolated” refers to a nucleic acid separated or significantly free from at least some of the other components of the naturally occurring organism, for example, the cell structural components or viral components commonly found associated with nucleic acids in the environment of the virus and/or other nucleic acids. The isolation of the native nucleic acids can be accomplished, for example, by techniques such as cell lysis followed by phenol plus chloroform extraction, followed by ethanol precipitation of the nucleic acids. The nucleic acids of this invention can be isolated from cells according to any of many methods well known in the art.

The AAV vectors disclose herein can comprise a heterologous nucleic acid functionally linked to the promoter. The term “heterologous” is used herein to refer to a nucleic acid which is derived from a different cell, tissue or organism. The nucleic acid can encode a polypeptide or protein or an antisense RNA, for example. By “functionally linked” is meant such that the promoter can promote expression of the heterologous nucleic acid, as is known in the art, such as appropriate orientation of the promoter relative to the heterologous nucleic acid. Furthermore, the heterologous nucleic acid preferably has all appropriate sequences for expression of the nucleic acid, as known in the art, to functionally encode, i.e., allow the nucleic acid to be expressed. The nucleic acid can include, for example, expression control sequences, such as an enhancer, and necessary information processing sites, such as ribosome binding sites, RNA splice sites, polyadenylation sites, and transcriptional terminator sequences.

The heterologous nucleic acid can encode beneficial proteins that replace missing or defective proteins required by the subject into which the vector in transferred or can encode a cytotoxic polypeptide that can be directed, e.g., to cancer cells or other cells whose death would be beneficial to the subject. The heterologous nucleic acid can also encode antisense RNAs that can bind to, and thereby inactivate, mRNAs made by the subject that encode harmful proteins. In one embodiment, antisense polynucleotides can be produced from a heterologous expression cassette in an AAV4 viral construct where the expression cassette contains a sequence that promotes cell-type specific expression (Wirak et al., 1991. EMBO 10:289). For general methods relating to antisense polynucleotides, see Antisense RNA and DNA, D. A. Melton, Ed., Cold Spring Harbor Laboratory, Cold Spring Harbor, N.Y. (1988).

Examples of heterologous nucleic acids which can be administered to a cell or subject as part of the present AAV4 vector can include, but are not limited to the following: nucleic acids encoding therapeutic agents, such as tumor necrosis factors (TNF), such as TNF-α; interferons, such as interferon-α interferon-β, and interferon-γ; interleukins, such as IL-1, IL-1β, and ILs-2 through -14; GM-CSF; adenosine deaminase; cellular growth factors, such as lymphokines; soluble CD4; Factor VIII; Factor IX; T-cell receptors; LDL receptor; ApoE; ApoC; alpha-1 antitrypsin; ornithine transcarbamylase (OTC); cystic fibrosis transmembrane receptor (CFTR); insulin; Fc receptors for antigen binding domains of antibodies, such as immunoglobulins; and antisense sequences which inhibit viral replication, such as antisense sequences which inhibit replication of hepatitis B or hepatitis non-A, non-B virus. The nucleic acid is chosen considering several factors, including the cell to be transfected. Where the target cell is a blood cell, for example, particularly useful nucleic acids to use are those which allow the blood cells to exert a therapeutic effect, such as a gene encoding a clotting factor for use in treatment of hemophilia. Furthermore, the nucleic acid can encode more than one gene product, limited only, if the nucleic acid is to be packaged in a capsid, by the size of nucleic acid that can be packaged.

The term “polypeptide” as used herein refers to a polymer of amino acids and includes full-length proteins and fragments thereof. Thus, “protein,” polypeptide,” and “peptide” are often used interchangeably herein. Substitutions can be selected by known parameters to be neutral (see, e.g., Robinson W E Jr, and Mitchell W M., 1990. AIDS 4:S151-S162). As will be appreciated by those skilled in the art, the invention also includes those polypeptides having slight variations in amino acid sequences or other properties. Such variations may arise naturally as allelic variations (e.g., due to genetic polymorphism) or may be produced by human intervention (e.g., by mutagenesis of cloned DNA sequences), such as induced point, deletion, insertion and substitution mutants. Minor changes in amino acid sequence are generally preferred, such as conservative amino acid replacements, small internal deletions or insertions, and additions or deletions at the ends of the molecules. Substitutions may be designed based on, for example, the model of Dayhoff, et al. (in Atlas of Protein Sequence and Structure 1978, Nat'l Biomed. Res. Found., Washington, D.C.). These modifications can result in changes in the amino acid sequence, provide silent mutations, modify a restriction site, or provide other specific mutations.

The term “epithelia” is used herein to refer to cells which are linked tightly together by intercellular junctions to form a planar sheet. These sheets of cells form a barrier between two compartments. Epithelia therefore line all surfaces and cavities (including skin, peritoneum, linings of the intestine, airways, genitourinary tracts, glands, and blood vessels.

An epithelium has a free or apical surface facing the environment, or lumen of a cavity, and a basal surface facing the underlying connective tissue. The boundary between the basal surface of an epithelium and the underlying connective tissue is usually very sharp, and is the site where the basal lamina (BL) is present. Most BL are too thin to be seen with the light microscope. However, the BL, together with a thin layer of connective tissue, is often times seen at the epithelial/connective tissue interface. This composite layer, visible with the light microscope, was initially called the Basement Membrane. Application of the electron microscope revealed that, in most cases, this Basement Membrane actually consisted of the true basal lamina (lamina lucida plus lamina densa), along with a layer of adherent connective tissue.

For convenience of description, epithelia are classified into different types based on the number of cell layers and the cell shape.

Epithelia which are 1 cell layer thick are called “simple” epithelia. Thus, each cell rests on the basal lamina, but also has a surface facing the lumen/outside world. Epithelia which are 2 or more cell layers thick are called “stratified” epithelia. In stratified epithelia, the basal layer of cells rests on the basal lamina, but subsequent layers do not, and are simply stacked on top of the basal layer. The cells of the most superficial layer have a free surface. “squamous” cells are very flat, like a fried egg, where the yolk is the nucleus. The nucleus is distinctly flattened, the cell is often so thin that this flattened nucleus bulges the cell surface outward. “cuboidal” cells range from true cuboidal where the cell is about as high as it is wide, to a flattened cuboidal where the cell is wider than high. In cuboidal cells the nucleus is usually round, and not flattened as in squamous. “columnar” cells are 2 or more times as high as wide. Nucleus is usually elongated in the long axis of the cell. Squamous cells form the lining of cavities such as the mouth, blood vessels, heart and lungs and make up the outer layers of the skin. Cuboidal epithelium is found in glands and in the lining of the kidney tubules as well as in the ducts of the glands. They also constitute the germinal epithelium which produces the egg cells in the female ovary and the sperm cells in the male testes. Columnar epithelium forms the lining of the stomach and intestines. Some columnar cells are specialized for sensory reception such as in the nose, ears and the taste buds of the tongue.

Ciliated columnar epithelial cells possess fine hair-like outgrowths, cilia on their free surfaces. These cilia are capable of rapid, rhythmic, wavelike beatings in a certain direction. Ciliated epithelium is usually found in the air passages like the nose. It is also found in the uterus and Fallopian tubes of females.

Columnar epithelium with goblet cells is called glandular epithelium. Some parts of the glandular epithelium consist of such a large number of goblet cells that there are only a few normal epithelial cells left. Columnar and cuboidal epithelial cells often become specialized as gland cells which are capable of synthesizing and secreting certain substances such as enzymes, hormones, milk, mucus, sweat, wax and saliva. Unicellular glands consist of single, isolated glandular cells such as the goblet cells. Sometimes a portion of the epithelial tissue becomes invaginated and a multicellular gland is formed. Multicellular glands are composed of clusters of cells. Most glands are multicellular including the salivary glands.

Where body linings have to withstand wear and tear, the epithelia are composed of several layers of cells and are then called compound or stratified epithelium. The top cells are flat and scaly and it may or may not be keratinized (i.e. containing a tough, resistant protein called keratin). The mammalian skin is an example of dry, keratinized, stratified epithelium. The lining of the mouth cavity is an example of an unkeratinized, stratified epithelium.

### In Vitro Cell Models of Transcytosis

The use of in vitro cell models to study transcytosis has many advantages over in vivo systems. First, variation among animals is eliminated, as is the confounding issue of cargo possibly being modified or endocytosed by cell types other than the one under study. Moreover, in vitro systems can be manipulated in ways not possible in vivo, allowing investigators to measure the effects of different variables (e.g., temperatures, pharmacological agents, etc.) with greater precision and to explore the molecular mechanisms of transcytosis.

The integrity of the monolayer is obviously vital to every study of transcytosis, and there are different methods for assessing it. Transepithelial electrical resistance (TER) measurements are commonly used as an indication of tight junction integrity in a monolayer, and commercial instruments are available for these measurements.

Caco-2 cells, human primary colon carcinoma cells, are a well studied model of intestinal absorptive enterocytes. They are the most commonly used intestinal cell line because they differentiate furthest along the cryptto-villus axis and are the easiest to transfect. Caco-2 cells have been especially used to model transcytosis of bacteria, which can cross barrier epithelia in the gut and brain (Zhang J R, et al., 2000. Cell 102(6):827-37), incorporated herein by reference.

There is little evidence for in vivo transcytosis of macromolecular cargo in kidney. Nonetheless, MDCK cells, which are derived from dog kidney, are the most-studied epithelial cell model and have been used extensively to study transcytosis. These cells were originally developed by nephrologists for permeability and electrical studies. Their subsequent use by cell biologists for studies of the formation of tight junctions, establishment of polarity, and vesicle traffic have popularized MDCK cells. An advantage is that MDCK cells are easily cultured, easily transfected, and become polarized 3-5 days after seeding. They were used in the now classical studies showing that enveloped viruses bud in a polarized fashion and that the newly synthesized viral membrane glycoproteins are targeted directly from the TGN to the appropriate PM domain. Furthermore, much of the current understanding of the IgA transcytotic pathway and the sorting signals in the pIgA-R comes from the elegant studies performed in MDCK cells. Two MDCK strains with very different features were identified some time ago. The MDCK I cell has a high TER and characteristics reminiscent of the renal collecting duct, whereas the more commonly used MDCK II strain, whose TER is one order of magnitude lower than that of MDCK I cells, has phenotypic features closer to those of the renal proximal tubule.

Both primary cells and cell lines, alone and in coculture with endothelial cells, are being used to study transcytosis in the lung. Clonetics bronchial/tracheal epithelial cell systems contain normal human bronchial/treacheal epithelial cells. This cell system has been used for experimental applications in cancer research, respiratory disease, cellular function and differentiation.

The Clonetics® bovine Brain Microvascular Endothelial Cell System (bMVEC-B) is a model of the “Blood Brain Barrier”. The system is designed to significantly improve a researcher's ability to study active and passive transport of drugs across the blood brain barrier, to study brain endothelial cell tight junctions, and to study the basic biology of brain microvascular endothelial cells (Schinket A H, 1999. Advanced Drug Delivery Reviews 36:179-194; Tsukita S. et al., 1998. Molecular dissection of tight junctions: occluding and ZO-1 in Introduction to the Blood—Brain Barrier. Edited by William M Partridge; Inglis et al., 2004. Brain Research 998: 218-229), each of which is incorporated by reference for its teaching of in vitro endothelial cell modeling of the blood-brain barrier.

Endometrial cells form an important barrier layer in the genitourinary tract. The cells used to model this system were developed by Kyo et al. and are derived from primary cells immortalized by the addition of the papillomiavirus E6/E7 genes and human telomerase reverse transcriptase. The isolated cells have a normal chromosomes and retain their responsiveness to sex-steriod hormones, exhibit glandular structure on three dimensional culture, and lack a transformed phenotype (Kyo S, et al. Am J Pathol., 2003. 163(6):2259-69), incorporated herein by reference for its teaching of this endometrial model.

### Methods of Use

The use of AAVs to deliver genes to the lung would be of benefit in genetic diseases like cystic fibrosis, pseudohypoaldosteronism, and immotile cilia syndrome. Furthermore, delivering genes to the lung would be of impact in several non-genetic diseases. For example, delivering genes that make antibiotic like peptides to the cells underlying the epithelia would be useful to prevent or treat bronchitis; delivering genes that make growth factors would be of value in common diseases like chronic bronchitis. Also, AAVs could be used to deliver genes that may play a role in asthma, like IL-10, or antibodies to IgE and interleukins. The use of an AAV vector to deliver genes through the alveolar epithelia would be of benefit in genetic diseases like alpha-1-antitrypsin deficiency. Furthermore, delivering genes through the alveolar epithelia would be of significance in several pulmonary non-genetic diseases. For example, delivering genes that make antibiotic like peptides would be useful to prevent or treat pneumonia (perhaps of antibiotic-resistant organisms); delivering genes that make growth factors would be of value in emphysema; delivering genes that-over-express the epithelial sodium channel or the Na-K ATPase could be used to treat cardiogenic and non-cardiogenic pulmonary edema; delivering genes that have an anti-fibrosis effect like interferon for pulmonary fibrosis would also be useful. Also, AAVs could be used to deliver genes that may have a systemic effect like anti-hypertension drugs, insulin, coagulation factors, antibiotics, growth factors, hormones and others.

The use of AAVs to deliver genes to the central nervous system (CNS)/brain would be of benefit in neurological diseases, including Alzheimer's Disease, Parkinson's Disease, Huntington's Disease, Tourette Syndrome, schizophrenia, mania, dementia, paranoia, obsessive compulsive disorder, panic disorder, learning disabilities, ALS, triplet expansions diseases, psychoses, autism, lysosomal storage diseases, Gaucher's disease, Hurler's disease, Krabbe's disease, battens disease, and altered behaviors (e.g., disorders in feeding, sleep patterns, balance, and perception).

The use of AAVs to deliver genes to the gastrointestinal system/gut would be of benefit in treatment of diseases and/or Gastrointestinal Disorders such as colon cancers, inflammatory bowel disease, diabetes, or Crohn's disease.

The use of AAVs to deliver genes to the genitourinary system would be of benefit in treatment of diseases of the female reproductive tract, molecular defects in implantation disorders, and gynecological cancers. These methods would also have contraceptive applications.

The use of AAVs to deliver genes to the kidney would be of benefit in treatment of inherited renal disorders such as polycystic kidney disease, Alport's syndrome, hereditary nephritis, primary hyperoxaluria, and cystinuria.

The use of AAVs for wide-spread delivery of genes across blood vessels into the muscle would be of benefit in neuromuscular diseases like muscular dystrophy and Cardiovascular Disorders such as heart disease, restenosis, atherosclerosis, myocarditis, stoke, angina, or thrombosis.

The use of AAVs for wide-spread delivery of genes across blood vessels into any/all tissues of a subject would be of benefit in the treatment of certain cancers (e.g., gastric, ovarian, lung, bladder, liver, and breast).

The use of AAVs for wide-spread delivery of genes across blood vessels into any/all tissues of a subject would be of benefit in the treatment of certain inflammatory disorders, including, but not limited to, adrenalitis, alveolitis, angiocholecystitis, appendicitis, balanitis, blepharitis, bronchitis, bursitis, carditis, cellulitis, cervicitis, cholecystitis, chorditis, cochlitis, colitis, conjunctivitis, cystitis, dermatitis, diverticulitis, encephalitis, endocarditis, esophagitis, eustachitis, fibrositis, folliculitis, gastritis, gastroenteritis, gingivitis, glossitis, hepatosplenitis, keratitis, labyrinthitis, laryngitis, lymphangitis, mastitis, media otitis, meningitis, metritis, mucitis, myocarditis, myosititis, myringitis, nephritis, neuritis, orchitis, osteochondritis, otitis, pericarditis, peritendonitis, peritonitis, pharyngitis, phlebitis, poliomyelitis, prostatitis, pulpitis, retinitis, rhinitis, salpingitis, scleritis, sclerochoroiditis, scrotitis, sinusitis, spondylitis, steatitis, stomatitis, synovitis, syringitis, tendonitis, tonsillitis, urethritis, and vaginitis; and disorders that are characterized by inflammation such as hepatitis, rheumatoid arthritis, gout, trauma, pancreatitis, sarcoidosis, dermatitis, renal ischemia-reperfusion injury, Grave's disease, systemic lupus erythematosus, diabetes mellitus, and allogenic transplant rejection.

The use of AAVs for wide-spread delivery of genes across blood vessels into any/all tissues of a subject would be of benefit in the treatment of other diseases, syndromes and conditions, such as adenosine deaminase deficiency, sickle cell deficiency, thalassemia, hemophilia, diabetes, phenylketonuria, growth disorders, and defects of the immune system.

### BAAV

Disclosed is a method of delivering a heterologous nucleic acid across an epithelial barrier of the lung, comprising delivering to the lung a BAAV vector comprising the nucleic acid. In one aspect of the method, the epithelial barrier comprises human bronchial, alveolar, tracheal or upper airway epithelial cells. Thus, disclosed is a method of delivering a heterologous nucleic acid across human airway epithelial cells, comprising delivering to the cells a BAAV vector comprising the nucleic acid.

Disclosed is a method of delivering a heterologous nucleic acid across an epithelial barrier in the brain, comprising delivering to the brain a BAAV vector comprising the nucleic acid. In one aspect of the method, the epithelial barrier comprises human cerebral microvascular endothelial cells or Choroidal Plexus epithelial cells of the blood brain barrier. Thus, disclosed is a method of delivering a heterologous nucleic acid across human cerebral microvascular endothelial cells, comprising delivering to the cells a BAAV vector comprising the nucleic acid.

Disclosed is a method of delivering a heterologous nucleic acid across the epithelial barrier of blood vessels into the muscle, comprising delivering to the blood stream a BAAV vector comprising the nucleic acid. In one aspect of the method, the epithelial barrier comprises human vascular endothelial cells.

Disclosed is a method of delivering a heterologous nucleic acid across an epithelial barrier in the genitourinary tract, comprising delivering to the genitourinary tract a BAAV vector comprising the nucleic acid genitourinary tract. In one aspect of the method, the epithelial barrier comprises human endometrial or urinary epithelial cells. Thus, disclosed is a method of delivering a heterologous nucleic acid across human endometrial epithelial cells, comprising delivering to the cells a BAAV vector comprising the nucleic acid.

Disclosed is a method of delivering a heterologous nucleic acid across an epithelial barrier in the kidney, comprising delivering to the genitourinary tract a BAAV vector comprising the nucleic acid genitourinary tract. In one aspect of the method, the epithelial barrier comprises human renal collecting ducts or proximal tubules. Thus, disclosed is a method of delivering a heterologous nucleic acid across human kidney epithelial cells, comprising delivering to the cells a BAAV vector comprising the nucleic acid.

Disclosed is a method of transcytosing lung epithelial cells of a subject comprising contacting the lung epithelial cells of the subject with a BAAV vector comprising a heterologous nucleic acid. In one aspect of the method, the epithelial cells are human bronchial, tracheal, or upper airway epithelial cells.

Disclosed is a method of transcytosing CNS epithelial cells of a subject comprising contacting the CNS epithelial cells of the subject with a BAAV vector comprising a heterologous nucleic acid. In one aspect of the method, the epithelial cells are human cerebral microvascular endothelial cells or Choroidal Plexus epithelial cells of the blood brain barrier.

Disclosed is a method of transcytosing vascular epithelial cells of a subject comprising contacting the vascular epithelial cells of the subject with a BAAV vector comprising a heterologous nucleic acid. In one aspect of the method, the epithelial cells are human vascular endothelial cells of the blood brain barrier.

Disclosed is a method of transcytosing genitourinary tract epithelial cells of a subject comprising contacting the genitourinary tract epithelial cells of the subject with a BAAV vector comprising a heterologous nucleic acid. In one aspect of the method, the epithelial cells are human endometrial or urinary tract epithelial cells.

Disclosed is a method of transcytosing genitourinary tract epithelial cells of a subject comprising contacting the kidney epithelial cells of the subject with a BAAV vector comprising a heterologous nucleic acid. In one aspect of the method, the epithelial cells are human renal collecting ducts or proximal tubules

### AAV5

Disclosed is a method of delivering a heterologous nucleic acid across an epithelial barrier in the gut, comprising delivering to the gut an AAV5 vector comprising the nucleic acid. In one aspect of the method, the epithelial barrier comprises human absorptive enterocytes or M cells. Thus, disclosed is a method of delivering a heterologous nucleic acid across human gut epithelial cells enterocytes, comprising delivering to the cells an AAV5 vector comprising the nucleic acid.

Disclosed is a method of transcytosing gut epithelial cells of a subject comprising contacting the gut epithelial cells of the subject with an AAV5 vector comprising a heterologous nucleic acid. In one aspect of the method, the epithelial cells are human absorptive enterocytes.

### AAV4

Disclosed is a method of delivering a heterologous nucleic acid across an epithelial barrier in the gut, comprising delivering to the gut an AAV4 vector comprising the nucleic acid. In one aspect of the method, the epithelial barrier comprises human absorptive enterocytes or M cells. Thus, disclosed is a method of delivering a heterologous nucleic acid across human gut epithelial cells enterocytes, comprising delivering to the cells an AAV4 vector comprising the nucleic acid.

Disclosed is a method of delivering a heterologous nucleic acid across an epithelial barrier in the lung, comprising delivering to the lung an AAV4 vector comprising the nucleic acid. In one aspect of the method, the epithelial barrier comprises human bronchial, tracheal, or upper airway epithelial cells. Thus, disclosed is a method of delivering a heterologous nucleic acid across human airway epithelial cells, comprising delivering to the cells an AAV4 vector comprising the nucleic acid.

Disclosed is a method of delivering a heterologous nucleic acid across an epithelial barrier in the CNS, comprising delivering to the CNS an AAV4 vector comprising the nucleic acid. In one aspect of the method, the epithelial barrier comprises human cerebral microvascular endothelial cells or Choroidal Plexus epithelial cells of the blood brain barrier. Thus, disclosed is a method of delivering a heterologous nucleic acid across human cerebral microvascular endothelial cells, comprising delivering to the cells a AAV4 vector comprising the nucleic acid.

Disclosed is a method of delivering a heterologous nucleic acid across the epithelial barrier of blood vessels into the muscle, comprising delivering to the blood stream an AAV4 vector comprising the nucleic acid. In one aspect of the method, the epithelial barrier comprises human vascular endothelial cells of the blood brain barrier.

Disclosed is a method of delivering a heterologous nucleic acid across an epithelial barrier in the genitourinary tract, comprising delivering to the genitourinary tract an AAV4 vector comprising the nucleic acid. In one aspect of the method, the epithelial barrier comprises human endometrial or urinary epithelial cells. Thus, disclosed is a method of delivering a heterologous nucleic acid across human endometrial epithelial cells, comprising delivering to the cells an AAV4 vector comprising the nucleic acid.

Disclosed is a method of delivering a heterologous nucleic acid across an epithelial barrier in the kidneys, comprising delivering to the kidneys an AAV4 vector comprising the nucleic acid. In one aspect of the method, the epithelial barrier comprises human renal collecting ducts or proximal tubules. Thus, disclosed is a method of delivering a heterologous nucleic acid across human kidney epithelial cells, comprising delivering to the cells an AAV4 vector comprising the nucleic acid.

Disclosed is a method of transcytosing lung epithelial cells of a subject comprising contacting the lung epithelial cells of the subject with an AAV4 vector comprising a heterologous nucleic acid. In one aspect of the method, the epithelial cells are human bronchial, tracheal, or upper airway epithelial cells.

Disclosed is a method of transcytosing CNS epithelial cells of a subject comprising contacting the CNS epithelial cells of the subject with an AAV4 vector comprising a heterologous nucleic acid. In one aspect of the method, the epithelial cells are human cerebral microvascular endothelial cells or Choroidal Plexus epithelial cells of the blood brain barrier.

Disclosed is a method of transcytosing vascular epithelial cells of a subject comprising contacting the vascular epithelial cells of the subject with an AAV4 vector comprising a heterologous nucleic acid. In one aspect of the method, the epithelial cells are vascular endothelial cells of the blood brain barrier.

Disclosed is a method of transcytosing genitourinary tract epithelial cells of a subject comprising contacting the genitourinary epithelial cells of the subject with an AAV4 vector comprising a heterologous nucleic acid. In one aspect of the method, the epithelial cells are human endometrial or urinary epithelial cells.

Disclosed is a method of transcytosing kidney epithelial cells of a subject comprising contacting the kidney epithelial cells of the subject with an AAV4 vector comprising a heterologous nucleic acid. In one aspect of the method, the epithelial cells are human renal collecting ducts or proximal tubules

Disclosed is a method of transcytosing gut epithelial cells of a subject comprising contacting the gut epithelial cells of the subject with an AAV4 vector comprising a heterologous nucleic acid. In one aspect of the method, the epithelial cells are human absorptive enterocytes.

### AAV7

Disclosed is a method of delivering a heterologous nucleic acid across an epithelial barrier in the CNS, comprising delivering to the CNS an AAV7 vector comprising the nucleic acid. In one aspect of the method, the epithelial barrier comprises human cerebral microvascular endothelial cells or Choroidal Plexus epithelial cells of the blood brain barrier. Thus, disclosed is a method of delivering a heterologous nucleic acid across human cerebral microvascular endothelial cells, comprising delivering to the cells an AAV7 vector comprising the nucleic acid.

Disclosed is a method of transcytosing CNS epithelial cells of a subject comprising contacting the CNS epithelial cells of the subject with an AAV7 vector comprising a heterologous nucleic acid. In one aspect of the method, the epithelial cells are human cerebral microvascular endothelial cells or Choroidal Plexus epithelial cells of the blood brain barrier.

### Inhibition of Transcytosis to Increase Transduction

Described herein is a method for re-directing virus that enters a cell by transcytosis to result in transduction of the cell by blocking exocytosis. Thus, provided is a method of improving the efficiency of nucleic acid delivery to epithelial cells, comprising delivering to the cells an inhibitor of exocytosis and an AAV vector containing the nucleic acid. Also provided is a method for transducing cells that have transcytosis activity but are normally resistant to transduction comprising administering to the cells inhibitors of exocytosis.

In one aspect of the methods, the AAV vector is derived from AAV4, AAV5, or BAAV. In a further aspect of the methods, the epithelial cell barriers are located in the kidney, gut, lung or vascular endothelium

Thus, disclosed is a method of delivering a heterologous nucleic acid to human airway epithelial cells, comprising delivering to the cells and an inhibitor of exocytosis and an AAV4 vector comprising the nucleic acid.

Further disclosed is a method of delivering a heterologous nucleic acid to human kidney epithelial cells, comprising delivering to the cells and an inhibitor of exocytosis and an AAV4 vector comprising the nucleic acid.

Further disclosed is a method of delivering a heterologous nucleic acid to human vascular endothelial cells, comprising delivering to the cells and an inhibitor of exocytosis and an AAV4 vector comprising the nucleic acid.

Further disclosed is a method of delivering a heterologous nucleic acid to human airway epithelial cells, comprising delivering to the cells and an inhibitor of exocytosis and a BAAV vector comprising the nucleic acid.

Further disclosed is a method of delivering a heterologous nucleic acid to human kidney epithelial cells, comprising delivering to the cells and an inhibitor of exocytosis and a BAAV vector comprising the nucleic acid.

Further disclosed is a method of delivering a heterologous nucleic acid to human vascular endothelial cells, comprising delivering to the cells and an inhibitor of exocytosis and a BAAV vector comprising the nucleic acid.

Further disclosed is a method of delivering a heterologous nucleic acid to human gut epithelial cells, comprising delivering to the cells and an inhibitor of exocytosis and an AAV5 vector comprising the nucleic acid.

In one aspect of the disclosed methods, the inhibitors of exocytosis are chemical modifiers. In a further aspect of the methods, the chemical modifier is tannic acid, wherein the tannic acid is delivered to the basal lateral surface of the epithelial cells.

### Compositions and Methods for Making AAV4 Vectors

Compositions and methods for making and using AAV4 vectors have been previously described in U.S. Pat. No. 6,468,524, which is hereby incorporated herein by reference for this teaching.

Provided is the nucleotide sequence of the adeno-associated virus 4 (AAV4) genome and vectors and particles derived therefrom. Specifically, provided is a nucleic acid vector comprising a pair of AAV4 inverted terminal repeats (ITRs) and a promoter between the inverted terminal repeats. The AAV4 ITRs are exemplified by the nucleotide sequence set forth in SEQ ID NO:6 and SEQ ID NO:20; however, these sequences can have minor modifications and still be contemplated to constitute AAV4 ITRs. The nucleic acid listed in SEQ ID NO:6 depicts the ITR in the “flip” orientation of the ITR. The nucleic acid listed in SEQ ID NO:20 depicts the ITR in the “flop” orientation of the ITR. Minor modifications in an ITR of either orientation are those that will not interfere with the hairpin structure formed by the AAV4 ITR as described herein and known in the art. Furthermore, to be considered within the term “AAV4 ITRs” the nucleotide sequence must retain the Rep binding site described herein and exemplified in SEQ ID NO:6 and SEQ ID NO:20, i.e., it must retain one or both features described herein that distinguish the AAV4 ITR from the AAV2 ITR: (1) four (rather than three as in AAV2) “GAGC” repeats and (2) in the AAV4 ITR Rep binding site the fourth nucleotide in the first two “GAGC” repeats is a T rather than a C.

The promoter can be any desired promoter, selected by known considerations, such as the level of expression of a nucleic acid functionally linked to the promoter and the cell type in which the vector is to be used. Promoters can be an exogenous or an endogenous promoter. Promoters can include, for example, known strong promoters such as SV40 or the inducible metallothionein promoter, or an AAV promoter, such as an AAV p5 promoter. Additional examples of promoters include promoters derived from actin genes, immunoglobulin genes, cytomegalovirus (CMV), adenovirus, bovine papilloma virus, adenoviral promoters, such as the adenoviral major late promoter, an inducible heat shock promoter, respiratory syncytial virus, Rous sarcomas virus (RSV), etc. Specifically, the promoter can be AAV2 p5 promoter or AAV4 p5 promoter. More specifically, the AAV4 p5 promoter can be about nucleotides 130 to 291 of SEQ ID NO: 1. Additionally, the p5 promoter may be enhanced by nucleotides 1-130. Furthermore, smaller fragments of p5 promoter that retain promoter activity can readily be determined by standard procedures including, for example, constructing a series of deletions in the p5 promoter, linking the deletion to a reporter gene, and determining whether the reporter gene is expressed, i.e., transcribed and/or translated.

The present invention also contemplates any unique fragment of these AAV4 nucleic acids, including the AAV4 nucleic acids set forth in SEQ ID NOs: 1, 3, 5, 6, 7, 12-15, 17 and 19. Fragments can be, for example, at least about 30, 40, 50, 75, 100, 200 or 500 nucleotides in length. The nucleic acid can be single or double stranded, depending upon the purpose for which it is intended.

The present invention further provides an AAV4 Capsid polypeptide or a unique fragment thereof. AAV4 capsid polypeptide is encoded by ORF 2 of AAV4. Specifically, provided is an AAV4 Capsid protein comprising the amino acid sequence encoded by nucleotides 2260-4464 of the nucleotide sequence set forth in SEQ ID NO:1, or a unique fragment of such protein. The present invention also provides an AAV4 Capsid protein consisting essentially of the amino acid sequence encoded by nucleotides 2260-4464 of the nucleotide sequence set forth in SEQ ID NO:1, or a unique fragment of such protein. The present invention further provides the individual AAV4 coat proteins, VP1, VP2 and VP3. Thus, provided is an isolated polypeptide having the amino acid sequence set forth in SEQ ID NO:4 (VP1). The present invention additionally provides an isolated polypeptide having the amino acid sequence set forth in SEQ ID NO:16 (VP2). The present invention also provides an isolated polypeptide having the amino acid sequence set forth in SEQ ID NO:18 (VP3). By “unique fragment thereof” is meant any smaller polypeptide fragment encoded by any AAV4 capsid gene that is of sufficient length to be unique to the AAV4 Capsid protein. Substitutions and modifications of the amino acid sequence can be made as described above and, further, can include protein processing modifications, such as glycosylation, to the polypeptide. However, an AAV4 Capsid polypeptide including all three coat proteins will have at least about 63% overall homology to the polypeptide encoded by nucleotides 2260-4464 of the sequence set forth in SEQ ID NO: 1. The protein can have about 65%, about 70%, about 75%, about 80%, about 85%, about 90%, about 95% or even 100% homology to the amino acid sequence encoded by the nucleotides 4467of the sequence set forth in SEQ ID NO:1. An AAV4 VP2 polypeptide can have at least about 58%, about 60%, about 70%, about 80%, about 90% about 95% or about 100% homology to the amino acid sequence set forth in SEQ ID NO:16. An AAV4 VP3 polypeptide can have at least about 60%, about 70%, about 80%, about 90% about 95% or about 100% homology to the amino acid sequence set forth in SEQ ID NO:18.

The herein described AAV4 nucleic acid vector can be encapsidated in an AAV particle. In particular, it can be encapsidated in an AAV1 particle, an AAV2 particle, an AAV3 particle, an AAV4 particle, or an AAV5 particle by standard methods using the appropriate capsid proteins in the encapsidation process, as long as the nucleic acid vector fits within the size limitation of the particle utilized. The encapsidation process itself is standard in the art.

An AAV4 particle is a viral particle comprising an AAV4 capsid protein. An AAV4 capsid polypeptide encoding the entire VP1, VP2, and VP3 polypeptide can overall have at least about 63% homology to the polypeptide having the amino acid sequence encoded by nucleotides 2260-4464 set forth in SEQ ID NO:1 (AAV4 capsid protein). The capsid protein can have about 70% homology, about 75% homology, 80% homology, 85% homology, 90% homology, 95% homology, 98% homology, 99% homology, or even 100% homology to the protein having the amino acid sequence encoded by nucleotides 2260-4464 set forth in SEQ ID NO:1. The particle can be a particle comprising both AAV4 and AAV2 capsid protein, i.e., a chimeric protein. Variations in the amino acid sequence of the AAV4 capsid protein are contemplated herein, as long as the resulting viral particle comprising the AAV4 capsid remains antigenically or immunologically distinct from AAV2, as can be routinely determined by standard methods. Specifically, for example, ELISA and Western blots can be used to determine whether a viral particle is antigenically or immunologically distinct from AAV2. Furthermore, the AAV4 viral particle preferably retains tissue tropism distinction from AAV2, such as that exemplified in the examples herein, though an AAV4 chimeric particle comprising at least one AAV4 coat protein may have a different tissue tropism from that of an AAV4 particle consisting only of AAV4 coat proteins.

An AAV4 particle is a viral particle comprising an AAV4 capsid protein. An AAV4 capsid polypeptide encoding the entire VP1, VP2, and VP3 polypeptide can overall have at least about 63% homology to the polypeptide having the amino acid sequence encoded by nucleotides 2260-4467 set forth in SEQ ID NO:1 (AAV4 capsid protein). The capsid protein can have about 70% homology, about 75% homology, 80% homology, 85% homology, 90% homology, 95% homology, 98% homology, 99% homology, or even 100% homology to the protein having the amino acid sequence encoded by nucleotides 2260-4467 set forth in SEQ ID NO:1. The particle can comprise only VP1 and VP3 and still stably transduce cells. The particle can be a particle comprising both AAV4 and AAV2 capsid protein, i.e., a chimeric protein. Variations in the amino acid sequence of the AAV4 capsid protein are contemplated herein, as long as the resulting viral particle comprising the AAV4 capsid remains antigenically or immunologically distinct from AAV2, as can be routinely determined by standard methods. Specifically, for example, ELISA and Western blots can be used to determine whether a viral particle is antigenically or immunologically distinct from AAV2. Furthermore, the AAV4 viral particle preferably retains tissue tropism distinction from AAV2, such as that exemplified in the examples herein, though an AAV4 chimeric particle comprising at least one AAV4 coat protein may have a different tissue tropism from that of an AAV4 particle consisting only of AAV4 coat proteins.

The invention further provides an AAV4 particle containing, i.e., encapsidating, a vector comprising a pair of AAV2 inverted terminal repeats. The nucleotide sequence of AAV2 ITRs is known in the art. Furthermore, the particle can be a particle comprising both AAV4 and AAV2 capsid protein, i.e., a chimeric protein. The vector encapsidated in the particle can further comprise an exogenous nucleic acid inserted between the inverted terminal repeats.

The present invention further provides an isolated nucleic acid comprising the nucleotide sequence set forth in SEQ ID NO:1 (AAV4 genome). This nucleic acid, or portions thereof, can be inserted into other vectors, such as plasmids, yeast artificial chromosomes, or other viral vectors, if desired, by standard cloning methods. The present invention also provides an isolated nucleic acid consisting essentially of the nucleotide sequence set forth in SEQ ID NO:1. The nucleotides of SEQ ID NO:1 can have minor modifications and still be contemplated by the present invention. For example, modifications that do not alter the amino acid encoded by any given codon (such as by modification of the third, “wobble,” position in a codon) can readily be made, and such alterations are known in the art. Furthermore, modifications that cause a resulting neutral amino acid substitution of a similar amino acid can be made in a coding region of the genome. Additionally, modifications as described herein for the AAV4 components, such as the ITRs, the p5 promoter, etc. are contemplated in this invention.

The present invention additionally provides an isolated nucleic acid that selectively hybridizes with an isolated nucleic acid consisting essentially of the nucleotide sequence set forth in SEQ ID NO:1 (AAV4 genome). The present invention further provides an isolated nucleic acid that selectively hybridizes with an isolated nucleic acid comprising the nucleotide sequence set forth in SEQ ID NO:1 (AAV4 genome). By “selectively hybridizes” as used in the claims is meant a nucleic acid that specifically hybridizes to the particular target nucleic acid under sufficient stringency conditions to selectively hybridize to the target nucleic acid without significant background hybridization to a nucleic acid encoding an unrelated protein, and particularly, without detectably hybridizing to AAV2. Thus, a nucleic acid that selectively hybridizes with a nucleic acid of the present invention will not selectively hybridize under stringent conditions with a nucleic acid encoding a different protein, and vice versa. Therefore, nucleic acids for use, for example, as primers and probes to detect or amplify the target nucleic acids are contemplated herein. Nucleic acid fragments that selectively hybridize to any given nucleic acid can be used, e.g., as primers and or probes for further hybridization or for amplification methods (e.g., polymerase chain reaction (PCR), ligase chain reaction (LCR)). Additionally, for example, a primer or probe can be designed that selectively hybridizes with both AAV4 and a gene of interest carried within the AAV4 vector (i.e., a chimeric nucleic acid).

The present invention further provides an isolated nucleic acid encoding an adeno-associated virus 4 Rep protein. The AAV4 Rep proteins are encoded by open reading frame (ORF) 1 of the AAV4 genome. The AAV4 Rep genes are exemplified by the nucleic acid set forth in SEQ ID NO:3 (AAV4 ORF1), and include a nucleic acid consisting essentially of the nucleotide sequence set forth in SEQ ID NO:3 and a nucleic acid comprising the nucleotide sequence set forth in SEQ ID NO:3. The present invention also includes a nucleic acid encoding the amino acid sequence set forth in SEQ ID NO: 2 (polypeptide encoded by AAV4 ORF1). However, the present invention includes that the Rep genes nucleic acid can include any one, two, three, or four of the four Rep proteins, in any order, in such a nucleic acid. Furthermore, minor modifications are contemplated in the nucleic acid, such as silent mutations in the coding sequences, mutations that make neutral or conservative changes in the encoded amino acid sequence, and mutations in regulatory regions that do not disrupt the expression of the gene. Examples of other minor modifications are known in the art. Further modifications can be made in the nucleic acid, such as to disrupt or alter expression of one or more of the Rep proteins in order to, for example, determine the effect of such a disruption; such as to mutate one or more of the Rep proteins to determine the resulting effect, etc. However, in general, a modified nucleic acid encoding all four Rep proteins will have at least about 90%, about 93%, about 95%, about 98% or 100% homology to the sequence set forth in SEQ ID NO:3, and the Rep polypeptide encoded therein will have overall about 93%, about 95%, about 98%, about 99% or 100% homology with the amino acid sequence set forth in SEQ ID NO:2.

The present invention also provides an isolated nucleic acid that selectively hybridizes with a nucleic acid consisting essentially of the nucleotide sequence set forth in SEQ ID NO:3 and an isolated nucleic acid that selectively hybridizes with a nucleic acid comprising the nucleotide sequence set forth in SEQ ID NO:3. “Selectively hybridizing” is defined elsewhere herein.

The present invention also provides each individual AAV4 Rep protein and the nucleic acid encoding each. Thus provided is the nucleic acid encoding a Rep 40 protein, and in particular an isolated nucleic acid comprising the nucleotide sequence set forth in SEQ ID NO:12, an isolated nucleic acid consisting essentially of the nucleotide sequence set forth in SEQ ID NO:12, and a nucleic acid encoding the adeno-associated virus 4 Rep protein having the amino acid sequence set forth in SEQ ID NO:8. The present invention also provides the nucleic acid encoding a Rep 52 protein, and in particular an isolated nucleic acid comprising the nucleotide sequence set forth in SEQ ID NO:13, an isolated nucleic acid consisting essentially of the nucleotide sequence set forth in SEQ ID NO:13, and a nucleic acid encoding the adeno-associated virus 4 Rep protein having the amino acid sequence set forth in SEQ ID NO:9. The present invention further provides the nucleic acid encoding a Rep 68 protein, and in particular an isolated nucleic acid comprising the nucleotide sequence set forth in SEQ ID NO:14, an isolated nucleic acid consisting essentially of the nucleotide sequence set forth in SEQ ID NO:14, and a nucleic acid encoding the adeno-associated virus 4 Rep protein having the amino acid sequence set forth in SEQ ID NO:10. And, further, provided is the nucleic acid encoding a Rep 78 protein, and in particular an isolated nucleic acid comprising the nucleotide sequence set forth in SEQ ID NO:15, an isolated nucleic acid consisting essentially of the nucleotide sequence set forth in SEQ ID NO:15, and a nucleic acid encoding the adeno-associated virus 4 Rep protein having the amino acid sequence set forth in SEQ ID NO:11. As described elsewhere herein, these nucleic acids can have minor modifications, including silent nucleotide substitutions, mutations causing neutral amino acid substitutions in the encoded proteins, and mutations in control regions that do not or minimally affect the encoded amino acid sequence.

The present invention further provides a nucleic acid encoding the entire AAV4 Capsid polypeptide. Specifically, provided is a nucleic acid having the nucleotide sequence set for the nucleotides 2260-4467 of SEQ ID NO:1. Furthermore, provided is a nucleic acid encoding each of the three AAV4 coat proteins, VP1, VP2, and VP3. Thus, provided is a nucleic acid encoding AAV4 VP1, a nucleic acid encoding AAV4 VP2, and a nucleic acid encoding AAV4 VP3. Thus, provided is a nucleic acid encoding the amino acid sequence set forth in SEQ ID NO:4 (VP1); a nucleic acid encoding the amino acid sequence set forth in SEQ ID NO:16 (VP2), and a nucleic acid encoding the amino acid sequence set forth in SEQ ID NO:18 (VP3). The present invention also specifically provides a nucleic acid comprising SEQ ID NO:5 (VP1 gene); a nucleic acid comprising SEQ ID NO:17 (VP2 gene); and a nucleic acid comprising SEQ ID NO:19 (VP3 gene). The present invention also specifically provides a nucleic acid consisting essentially of SEQ ID NO:5 (VP1 gene), a nucleic acid consisting essentially of SEQ ID NO:17 (VP2 gene), and a nucleic acid consisting essentially of SEQ ID NO:19 (VP3 gene). Furthermore, a nucleic acid encoding an AAV4 capsid protein VP1 is set forth as nucleotides 2260-4467 of SEQ ID NO:1; a nucleic acid encoding an AAV4 capsid protein VP2 is set forth as nucleotides 2668-4467 of SEQ ID NO:1; and a nucleic acid encoding an AAV4 capsid protein VP3 is set forth as nucleotides 2848-4467 of SEQ ID NO:1. Minor modifications in the nucleotide sequences encoding the capsid, or coat, proteins are contemplated, as described above for other AAV4 nucleic acids.

Provided is an isolated AAV4 Rep protein. AAV4 Rep polypeptide is encoded by ORF1 of AAV4. Specifically, provided is an AAV4 Rep polypeptide comprising the amino acid sequence set forth in SEQ ID NO:2, or a unique fragment thereof. The present invention also provides an AAV4 Rep polypeptide consisting essentially of the amino acid sequence set forth in SEQ ID NO:2, or a unique fragment thereof. Additionally, nucleotides 291-2306 of the AAV4 genome, which genome is set forth in SEQ ID NO:1, encode the AAV4 Rep polypeptide. The present invention also provides each AAV4 Rep protein. Thus provided is AAV4 Rep 40, or a unique fragment thereof. The present invention particularly provides Rep 40 having the amino acid sequence set forth in SEQ ID NO:8. Provided is AAV4 Rep 52, or a unique fragment thereof. The present invention particularly provides Rep 52 having the amino acid sequence set forth in SEQ ID NO:9. Provided is AAV4 Rep 68, or a unique fragment thereof. The present invention particularly provides Rep 68 having the amino acid sequence set forth in SEQ ID NO:10. Provided is AAV4 Rep 78, or a unique fragment thereof. The present invention particularly provides Rep 78 having the amino acid sequence set forth in SEQ ID NO:11. By “unique fragment thereof” is meant any smaller polypeptide fragment encoded by AAV rep gene that is of sufficient length to be unique to the Rep polypeptide. Substitutions and modifications of the amino acid sequence can be made as described above and, further, can include protein processing modifications, such as glycosylation, to the polypeptide. However, a polypeptide including all four Rep proteins will encode a polypeptide having at least about 91% overall homology to the sequence set forth in SEQ ID NO:2, and it can have about 93%, about 95%, about 98%, about 99% or 100% homology with the amino acid sequence set forth in SEQ ID NO:2.

The present invention further provides an AAV4 Capsid polypeptide or a unique fragment thereof. AAV4 capsid polypeptide is encoded by ORF 2 of AAV4. Specifically, provided is an AAV4 Capsid protein comprising the amino acid sequence encoded by nucleotides 2260-4467 of the nucleotide sequence set forth in SEQ ID NO:1, or a unique fragment of such protein. The present invention also provides an AAV4 Capsid protein consisting essentially of the amino acid sequence encoded by nucleotides 2260-4467 of the nucleotide sequence set forth in SEQ ID NO:1, or a unique fragment of such protein. The present invention further provides the individual AAV4 coat proteins, VP1, VP2 and VP3. Thus, provided is an isolated polypeptide having the amino acid sequence set forth in SEQ ID NO:4 (VP1). The present invention additionally provides an isolated polypeptide having the amino acid sequence set forth in SEQ ID NO:16 (VP2). The present invention also provides an isolated polypeptide having the amino acid sequence set forth in SEQ ID NO:18 (VP3). By “unique fragment thereof” is meant any smaller polypeptide fragment encoded by any AAV4 capsid gene that is of sufficient length to be unique to the AAV4 Capsid protein. Substitutions and modifications of the amino acid sequence can be made as described above and, further, can include protein processing modifications, such as glycosylation, to the polypeptide. However, an AAV4 Capsid polypeptide including all three coat proteins will have at least about 63% overall homology to the polypeptide encoded by nucleotides 2260-4467 of the sequence set forth in SEQ ID NO: 1. The protein can have about 65%, about 70%, about 75%, about 80%, about 85%, about 90%, about 95% or even 100% homology to the amino acid sequence encoded by the nucleotides 2260-4467 of the sequence set forth in SEQ ID NO:4. An AAV4 VP2 polypeptide can have at least about 58%, about 60%, about 70%, about 80%, about 90% about 95% or about 100% homology to the amino acid sequence set forth in SEQ ID NO:16. An AAV4 VP3 polypeptide can have at least about 60%, about 70%, about 80%, about 90% about 95% or about 100% homology to the amino acid sequence set forth in SEQ ID NO:18.

The AAV inverted terminal repeats in the vector for the herein described delivery methods can be AAV4 inverted terminal repeats. Specifically, they can comprise the nucleic acid whose nucleotide sequence is set forth in SEQ ID NO:6 or the nucleic acid whose nucleotide sequence is set forth in SEQ ID NO:20, or any fragment thereof demonstrated to have ITR functioning. The ITRs can also consist essentially of the nucleic acid whose nucleotide sequence is set forth in SEQ ID NO:6 or the nucleic acid whose nucleotide sequence is set forth in SEQ ID NO:20. Furthermore, the AAV inverted terminal repeats in the vector for the herein described nucleic acid delivery methods can also comprise AAV2 inverted terminal repeats. Additionally, the AAV inverted terminal repeats in the vector for this delivery method can also consist essentially of AAV2 inverted terminal repeats.

### Compositions and Methods for Making AAV5 Vectors

Compositions and methods for making and using AAV5 vectors have been previously described in U.S. patent application Ser. No. 09/717,789, filed Nov. 21, 2000, and in U.S. Pat. No. 6,855,314, which are hereby incorporated herein by reference for this teaching.

The present application provides a recombinant adeno-associated virus 5 (AAV5). This virus has one or more of the characteristics described below. The compositions of the present invention do not include wild-type AAV5. The methods of the present invention can use either wild-type AAV5 or recombinant AAV5-based delivery.

Provided are novel AAV5 particles, recombinant AAV5 vectors, recombinant AAV5 virions and novel AAV5 nucleic acids and polypeptides. An AAV5 particle is a viral particle comprising an AAV5 capsid protein. A recombinant AAV5 vector is a nucleic acid construct that comprises at least one unique nucleic acid of AAV5. A recombinant AAV5 virion is a particle containing a recombinant AAV5 vector, wherein the particle can be either an AAV5 particle as described herein or a non-AAV5 particle. Alternatively, the recombinant AAV5 virion is an AAV5 particle containing a recombinant vector, wherein the vector can be either an AAV5 vector as described herein or a non-AAV5 vector. These vectors, particles, virions, nucleic acids and polypeptides are described below.

Provided is the nucleotide sequence of the AAV5 genome and vectors and particles derived therefrom. Specifically, provided is a nucleic acid vector comprising a pair of AAV5 inverted terminal repeats (ITRs) and a promoter between the inverted terminal repeats. While the rep proteins of AAV2 and AAV5 will bind to either a type 2 ITR or a type 5 ITR, efficient genome replication only occurs when type 2 Rep replicates a type 2 ITR and a type 5 Rep replicates a type 5 ITR. This specificity is the result of a difference in DNA cleavage specificity of the two Reps which is necessary for replication. AAV5 Rep cleaves at CGGT̂GTGA (SEQ ID NO: 43) and AAV2 Rep cleaves at CGGTATGAG (SEQ ID NO: 44) (Chiorini et al., 1999. J. Virol. 73 (5) 4293-4298). Mapping of the AAV5 ITR terminal resolution site (TRS) identified this distinct cleavage site, CGGT̂GTGA, which is absent from the ITRs of other AAV serotypes. Therefore, the minimum sequence necessary to distinguish AAV5 from AAV2 is the TRS site where Rep cleaves in order to replicate the virus. Examples of the type 5 ITRs are shown in SEQ ID NO: 41 and SEQ ID NO: 42, AAV5 ITR “flip” and AAV5 “flop”, respectively. Minor modifications in an ITR of either orientation are contemplated and are those that will not interfere with the hairpin structure formed by the AAV5 ITR as described herein. Furthermore, to be considered within the term “AAV5 ITR” the nucleotide sequence must retain one or more features described herein that distinguish the AAV5 ITR from the ITRs of other serotypes, e.g. it must retain the Rep binding site described herein.

The D− region of the AAV5 ITR (SEQ ID NO: 45), a single stranded region of the ITR, inboard of the TRS site, has been shown to bind a factor which depending on its phosphorylation state correlates with the conversion of the AAV from a single stranded genome to a transcriptionally active form that allows for expression of the viral DNA. This region is conserved between AAV2, 3, 4,and 6 but is divergent in AAV5. The D+ region is the reverse complement of the D− region.

The promoter can be any desired promoter, selected by known considerations, such as the level of expression of a nucleic acid functionally linked to the promoter and the cell type in which the vector is to be used. That is, the promoter can be tissue/cell-specific. Promoters can be prokaryotic, eukaryotic, fungal, nuclear, mitochondrial, viral or plant promoters. Promoters can be exogenous or endogenous to the cell type being transduced by the vector. Promoters can include, for example, bacterial promoters, known strong promoters such as SV40 or the inducible metallothionein promoter, or an AAV promoter, such as an AAV p5 promoter. Additionally, chimeric regulatory promoters for targeted gene expression can be utilized. Examples of these regulatory systems, which are known in the art, include the tetracycline based regulatory system which utilizes the tet transactivator protein (tTA), a chimeric protein containing the VP16 activation domain fused to the tet repressor of Escherichia coli, the IPTG based regulatory system, the CID based regulatory system, and the Ecdysone based regulatory system. Other promoters include promoters derived from actin genes, immunoglobulin genes, cytomegalovirus (CMV), adenovirus, bovine papilloma virus, adenoviral promoters, such as the adenoviral major late promoter, an inducible heat shock promoter, respiratory syncytial virus, Rous sarcomas virus (RSV), etc., specifically, the promoter can be AAV2 p5 promoter or AAV5 p5 promoter. More specifically, the AAV5 p5 promoter can be about same location in SEQ ID NO: 23 as the AAV2 p5 promoter, in the corresponding AAV2 published sequence. An example of an AAV5 p5 promoter is nucleotides 220-338 of SEQ ID NO: 23. Additionally, the p5 promoter may be enhanced by nucleotides 1-130 of SEQ ID NO: 23. Furthermore, smaller fragments of p5 promoter that retain promoter activity can readily be determined by standard procedures including, for example, constructing a series of deletions in the p5 promoter, linking the deletion to a reporter gene, and determining whether the reporter gene is expressed, i.e., transcribed and/or translated. The promoter can be the promoter of any of the AAV serotypes, and can be the p19 promoter (SEQ ID NO: 38) or the p40 promoter set forth in the sequence listing as SEQ ID NO: 39.

It should be recognized that any errors in any of the nucleotide sequences disclosed herein can be corrected, for example, by using the hybridization procedure described below with various probes derived from the described sequences such that the coding sequence can be reisolated and resequenced. Rapid screening for point mutations can also be achieved with the use of polymerase chain reaction single strand conformation polymorphism (PCR SSCP). The corresponding amino acid sequence can then be corrected accordingly.

The AAV5-derived vector can include any normally occurring AAV5 sequences in addition to an ITR and promoter. Examples of vector constructs are provided below.

The present vector or AAV5 particle or recombinant AAV5 virion can utilize any unique fragment of the present AAV5 nucleic acids, including the AAV5 nucleic acids set forth in SEQ ID NOS: 23 and 29-33, 35, 37, 38, 39 and 40. To be unique, the fragment must be of sufficient size to distinguish it from other known sequences, most readily determined by comparing any nucleic acid fragment to the nucleotide sequences of nucleic acids in computer databases, such as GenBank. Such comparative searches are standard in the art. Typically, a unique fragment useful as a primer or probe will be at least about 8 or 10, preferable at least 20 or 25 nucleotides in length, depending upon the specific nucleotide content of the sequence. Additionally, fragments can be, for example, at least about 30, 40, 50, 75, 100, 200 or 500 nucleotides in length and can encode polypeptides or be probes. The nucleic acid can be single or double stranded, depending upon the purpose for which it is intended. Where desired, the nucleic acid can be RNA.

The present invention further provides an isolated AAV5 capsid protein to contain the vector. In particular, provided is not only a polypeptide comprising all three AAV5 coat proteins, i.e., VP1, VP2 and VP3, but also a polypeptide comprising each AAV5 coat protein individually, SEQ ID NOS: 26, 27, and 28, respectively. Thus an AAV5 particle comprising an AAV5 capsid protein comprises at least one AAV5 coat protein VP1, VP2 or VP3. An AAV5 particle comprising an AAV5 capsid protein can be utilized to deliver a nucleic acid vector to a cell, tissue or subject. For example, the herein described AAV5 vectors can be encapsidated in an AAV5 capsid-derived particle and utilized in a gene delivery method. Furthermore, other viral nucleic acids can be encapsidated in the AAV5 particle and utilized in such delivery methods. For example, an AAV1, 2, 3, 4, or 6 vector (e.g. AAV1, 2, 3, 4, or 6 ITR and nucleic acid of interest)can be encapsidated in an AAV5 particle and administered. Furthermore, an AAV5 chimeric capsid incorporating both AAV2 capsid and AAV5 capsid sequences can be generated, by standard cloning methods, selecting regions from the known sequences of each protein as desired. For example, particularly antigenic regions of the AAV2 capsid protein can be replaced with the corresponding region of the AAV5 capsid protein. In addition to chimeric capsids incorporating AAV2 capsid sequences, chimeric capsids incorporating AAV1, 3, 4, or 6 and AAV5 capsid sequences can be generated, by standard cloning methods, selecting regions from the known sequences of each protein as desired. The particle can also comprise only VP1 and VP3 capsid proteins.

The capsids can also be modified to alter their specific tropism by genetically altering the capsid to encode a specific ligand to a cell surface receptor. Alternatively, the capsid can be chemically modified by conjugating a ligand to a cell surface receptor. By genetically or chemically altering the capsids, the tropism can be modified to direct AAV5 to a particular cell or population of cells. The capsids can also be altered immunologically by conjugating the capsid to an antibody that recognizes a specific protein on the target cell or population of cells.

The capsids can also be assembled into empty particles by expression in mammalian, bacterial, fungal or insect cells. For example, AAV2 particles are known to be made from VP3 and VP2 capsid proteins in baculovirus. The same basic protocol can produce an empty AAV5 particle comprising an AAV5 capsid protein.

The herein described recombinant AAV5 nucleic acid derived vector can be encapsidated in an AAV particle. In particular, it can be encapsidated in an AAV1 particle, an AAV2 particle, an AAV3 particle, an AAV4 particle, an AAV5 particle or an AAV6 particle, a portion of any of these capsids, or a chimeric capsid particle as described above, by standard methods using the appropriate capsid proteins in the encapsidation process, as long as the nucleic acid vector fits within the size limitation of the particle utilized. The encapsidation process itself is standard in the art. The AAV5 replication machinery, i.e. the rep initiator proteins and other functions required for replication, can be utilized to produce the AAV5 genome that can be packaged in an AAV1, 2, 3, 4, 5 or 6 capsid.

The recombinant AAV5 virion containing a vector can also be produced by recombinant methods utilizing multiple plasmids. In one example, the AAV5 rep nucleic acid would be cloned into one plasmid, the AAV5 ITR nucleic acid would be cloned into another plasmid and the AAV1, 2, 3, 4, 5 or 6 capsid nucleic acid would be cloned on another plasmid. These plasmids would then be introduced into cells. The cells that were efficiently transduced by all three plasmids, would exhibit specific integration as well as the ability to produce recombinant AAV5 virion. Additionally, two plasmids could be used where the AAV5 rep nucleic acid would be cloned into one plasmid and the AAV5 ITR and AAV5 capsid would be cloned into another plasmid. These plasmids would then be introduced into cells. The cells that were efficiently transduced by both plasmids, would exhibit specific integration as well as the ability to produce recombinant AAV5 virion.

An AAV5 capsid polypeptide encoding the entire VP1, VP2, and VP3 polypeptide can have greater than 56% overall homology to the polypeptide having the amino acid sequence encoded by nucleotides in SEQ ID NOS: 29, 30, 31, as shown in FIGS. 4 and 5. The capsid protein can have about 70% homology, about 75% homology, 80% homology, 85% homology, 90% homology, 95% homology, 98% homology, 99% homology, or even 100% homology to the protein having the amino acid sequence encoded by the nucleotides set forth in SEQ ID NOS: 29, 30, or 31. The percent homology used to identify proteins herein, can be based on a nucleotide-by-nucleotide comparison or more preferable is based on a computerized algorithm as described herein. Variations in the amino acid sequence of the AAV5 capsid protein are contemplated herein, as long as the resulting particle comprising an AAV5 capsid protein remains antigenic ally or immunologically distinct from AAV1, AAV2, AAV3, AAV4 or AAV6 capsid, as can be routinely determined by standard methods. Specifically, for example, ELISA and Western blots can be used to determine whether a viral particle is antigenically or immunologically distinct from AAV2 or the other serotypes. Furthermore, the AAV5 particle preferably retains tissue tropism distinction from AAV2, such as that exemplified in the examples herein. An AAV5 chimeric particle comprising at least one AAV5 coat protein may have a different tissue tropism from that of an AAV5 particle consisting only of AAV5 coat proteins, but is still distinct from the tropism of an AAV2 particle, in that it will infect some cells not infected by AAV2 or an AAV2 particle.

The invention further provides a recombinant AAV5 virion, comprising an AAV5 particle containing, i.e., encapsidating, a vector comprising a pair of AAV5 inverted terminal repeats. The recombinant vector can further comprise an AAV5 Rep-encoding nucleic acid. The vector encapsidated in the particle can further comprise an exogenous nucleic acid inserted between the inverted terminal repeats. AAV5 Rep confers targeted integration and efficient replication, thus production of recombinant AAV5, comprising AAV5 Rep, yields more particles than production of recombinant AAV2. Since AAV5 is more efficient at replicating and packaging its genome, the exogenous nucleic acid inserted, or in the AAV5 capsids of the present invention, between the inverted terminal repeats can be packaged in the AAV1, 2, 3, 4, or 6 capsids to achieve the specific tissue tropism conferred by the capsid proteins.

The invention further contemplates chimeric recombinant ITRs that contains a rep binding site and a TRS site recognized by that Rep protein. By “Rep protein” is meant all four of the Rep proteins, Rep 40, Rep 78, Rep 52, Rep 68. Alternatively, “Rep protein” could be one or more of the Rep proteins described herein. One example of a chimeric ITR would consist of an AAV5 D region (SEQ ID NO: 45), an AAV5 TRS site (SEQ ID NO: 43), an AAV2 hairpin and an AAV2 binding site. Another example would be an AAVS D region, an AAVS TRS site, an AAV3 hairpin and an AAV3 binding site. In these chimeric ITRs, the D region can be from AAV1, 2, 3, 4, 5 or 6. The hairpin can be derived from AAV 1, 2 3, 4, 5, 6. The binding site can be derived from any of AAV1, 2, 3, 4, 5 or 6. Preferably, the D region and the TRS are from the same serotype.

The chimeric ITRs can be combined with AAV5 Rep protein and any of the AAV serotype capsids to obtain recombinant virion. For example, recombinant virion can be produced by an AAV5 D region, an AAV5 TRS site, an AAV2 hairpin, an AAV2 binding site, AAV5 Rep protein and AAV1 capsid. This recombinant virion would possess the cellular tropism conferred by the AAV1 capsid protein and would possess the efficient replication conferred by the AAV5 Rep.

Other examples of the ITR, Rep protein and Capsids that will produce recombinant virion are provided in the list below:

5ITR+5Rep+5Cap=virion

5ITR+5Rep+1Cap=virion

5ITR+5Rep+2Cap=virion

5ITR+5Rep+3Cap=virion

5ITR+5Rep+4Cap=virion

5ITR+5Rep+6Cap=virion

1ITR+1Rep+5Cap=virion

2ITR+2Rep+5Cap=virion

3ITR+3Rep+5Cap=virion

4ITR+4Rep+5Cap=virion

6ITR+6Rep+5Cap=virion

In any of the constructs described herein, inclusion of a promoter is preferred. As used in the constructs herein, unless otherwise specified, Cap (capsid) refers to any of AAV5 VP1, AAV5 VP2, AAV5 VP3, combinations thereof, functional fragments of any of VP1, VP2 or VP3, or chimeric capsids as described herein. The ITRs of the constructs described herein, can be chimeric recombinant ITRs as described elsewhere in the application.

Conjugates of recombinant or wild-type AAV5 virions and nucleic acids or proteins can be used to deliver those molecules to a cell. For example, the purified AAV5 can be used as a vehicle for delivering DNA bound to the exterior of the virus. Examples of this are to conjugate the DNA to the virion by a bridge using poly L lysine or other charged molecule. Also contemplated are virosomes that contain AAV5 structural proteins (AAV5 capsid proteins), lipids such as DOTAP, and nucleic acids that are complexed via charge interaction to introduce DNA into cells.

Also provided by this invention are conjugates that utilize the AAV5 capsid or a unique region of the AAV5 capsid protein (e.g. VP1, VP2 or VP3 or combinations thereof) to introduce DNA into cells. For example, the type 5 VP3 protein or fragment thereof, can be conjugated to a DNA on a plasmid that is conjugated to a lipid. Cells can be infected using the targeting ability of the VP3 capsid protein to achieve the desired tissue tropism, specific to AAV5. Type 5 VP1 and VP2 proteins can also be utilized to introduce DNA or other molecules into cells. By further incorporating the Rep protein and the AAV TRS into the DNA-containing conjugate, cells can be transduced and targeted integration can be achieved. For example, if AAV5 specific targeted integration is desired, a conjugate composed of the AAV5 VP3 capsid, AAV5 rep or a fragment of AAV5 rep, AAV5 TRS, the rep binding site, the heterologous DNA of interest, and a lipid, can be utilized to achieve AAV5 specific tropism and AAV5 specific targeted integration in the genome.

Further provided by this invention are chimeric viruses where AAV5 can be combined with herpes virus, herpes virus amplicons, baculovirus or other viruses to achieve a desired tropism associated with another virus. For example, the AAV5 ITRs could be inserted in the herpes virus and cells could be infected. Post-infection, the ITRs of AAV5 could be acted on by AAV5 rep provided in the system or in a separate vehicle to rescue AAV5 from the genome. Therefore, the cellular tropism of the herpes simplex virus can be combined with AAV5 rep mediated targeted integration. Other viruses that could be utilized to construct chimeric viruses include, lentivirus, retrovirus, pseudotyped retroviral vectors, and adenoviral vectors.

The present invention further provides isolated nucleic acids of AAV5. For example, provided is an isolated nucleic acid comprising the nucleotide sequence set forth in SEQ ID NO: 23 (AAV5 genome). This nucleic acid, or portions thereof, can be inserted into vectors, such as plasmids, yeast artificial chromosomes, or other viral vector (particle), if desired, by standard cloning methods. The present invention also provides an isolated nucleic acid consisting essentially of the nucleotide sequence set forth in SEQ ID NO: 23. The nucleotides of SEQ ID NO: 23 can have minor modifications and still be contemplated by the present invention. For example, modifications that do not alter the amino acid encoded by any given codon (such as by modification of the third, “wobble,” position in a codon) can readily be made, and such alterations are known in the art. Furthermore, modifications that cause a resulting neutral (conserved) amino acid substitution of a similar amino acid can be made in a coding region of the genome. Additionally, modifications as described herein for the AAV5 components, such as the ITRs, the p5 promoter, etc. are contemplated in this invention. Furthermore, modifications to regions of SEQ ID NO: 23 other than in the ITR, TRS Rep binding site and hairpin are likely to be tolerated without serious impact on the function of the nucleic acid as a recombinant vector.

The present invention additionally provides an isolated nucleic acid that selectively hybridizes with any nucleic acid disclosed herein, including the entire AAV5 genome and any unique fragment thereof, including the Rep and capsid encoding sequences (e.g. SEQ ID NOS: 23, 29, 30, 31, 32, 33, 35, 36, 37, 38, 39, 40, 41, 42, 43, 44, and 45). Specifically, the nucleic acid can selectively or specifically hybridize to an isolated nucleic acid consisting of the nucleotide sequence set forth in SEQ ID NO: 23 (AAV5 genome). The present invention further provides an isolated nucleic acid that selectively or specifically hybridizes with an isolated nucleic acid comprising the nucleotide sequence set forth in SEQ ID NO: 23 (AAV5 genome). By “selectively hybridizes” as used herein is meant a nucleic acid that hybridizes to one of the disclosed nucleic acids under sufficient stringency conditions without significant hybridization to a nucleic acid encoding an unrelated protein, and particularly, without detectably hybridizing to nucleic acids of AAV2. Thus, a nucleic acid that selectively hybridizes with a nucleic acid of the present invention will not selectively hybridize under stringent conditions with a nucleic acid encoding a different protein or the corresponding protein from a different serotype of the virus, and vice versa. A “specifically hybridizing” nucleic acid is one that hybridizes under stringent conditions to only a nucleic acid found in AAV5. Therefore, nucleic acids for use, for example, as primers and probes to detect or amplify the target nucleic acids are contemplated herein. Nucleic acid fragments that selectively hybridize to any given nucleic acid can be used, e.g., as primers and or probes for further hybridization or for amplification methods (e.g., polymerase chain reaction (PCR), ligase chain reaction (LCR)). Additionally, for example, a primer or probe can be designed that selectively hybridizes with both AAV5 and a gene of interest carried within the AAV5 vector (i.e., a chimeric nucleic acid).

A nucleic acid that selectively hybridizes to any portion of the AAV5 genome is contemplated herein. Therefore, a nucleic acid that selectively hybridizes to AAV5 can be of longer length than the AAV5 genome, it can be about the same length as the AAV5 genome or it can be shorter than the AAV5 genome. The length of the nucleic acid is limited on the shorter end of the size range only by its specificity for hybridization to AAV5, i.e., once it is too short, typically less than about 5 to 7 nucleotides in length, it will no longer bind specifically to AAV5, but rather will hybridize to numerous background nucleic acids. Additionally contemplated by this invention is a nucleic acid that has a portion that specifically hybridizes to AAV5 and a portion that specifically hybridizes to a gene of interest inserted within AAV5.

The present invention further provides an isolated nucleic acid encoding an adeno-associated virus 5 Rep protein. The AAV5 Rep proteins are encoded by open reading frame (ORF) 1 of the AAV5 genome. Examples of the AAV5 Rep genes are shown in the nucleic acid set forth in SEQ ID NO: 23, and include nucleic acids consisting essentially of the nucleotide sequences set forth in SEQ ID NOS: 32 (Rep52), 33 (Rep78), 35 (Rep40), and 37 (Rep68), and nucleic acids comprising the nucleotide sequences set forth in SEQ ID NOS: 32, 33, 35, and 37. However, the present invention contemplates that the Rep nucleic acid can include any one, two, three, or four of the four Rep proteins, in any order, in such a nucleic acid. Furthermore, minor modifications are contemplated in the nucleic acid, such as silent mutations in the coding sequences, mutations that make neutral or conservative changes in the encoded amino acid sequence, and mutations in regulatory regions that do not disrupt the expression of the gene. Examples of other minor modifications are known in the art. Further modifications can be made in the nucleic acid, such as to disrupt or alter expression of one or more of the Rep proteins in order to, for example, determine the effect of such a disruption; such as to mutate one or more of the Rep proteins to determine the resulting effect, etc. However, in general, a modified nucleic acid encoding a Rep protein will have at least about 85%, about 90%, about 93%, about 95%, about 98% or 100% homology to the Rep nucleic sequences described herein e.g., SEQ ID NOS:, 11, 13 and 15 32, 33, 35 and 37, and the Rep polypeptide encoded therein will have overall about 93%, about 95%, about 98%, about 99% or 100% homology with the amino acid sequence described herein, e.g., SEQ ID NOS: 24, 25, 34 and 36. Percent homology is determined by the techniques described herein.

The present invention also provides an isolated nucleic acid that selectively or specifically hybridizes with a nucleic acid consisting essentially of the nucleotide sequence set forth in SEQ ID NOS: 32, 33, 35 and 37, and an isolated nucleic acid that selectively hybridizes with a nucleic acid comprising the nucleotide sequence set forth in SEQ ID NOS: 32, 33, 35 and 37. “Selectively hybridizing” and “stringency of hybridization” is defined elsewhere herein.

As described above, provided is the nucleic acid encoding a Rep 40 protein and, in particular an isolated nucleic acid comprising the nucleotide sequence set forth in SEQ ID NO: 35, an isolated nucleic acid consisting essentially of the nucleotide sequence set forth in SEQ ID NO: 35, and a nucleic acid encoding the adeno-associated virus 5 protein having the amino acid sequence set forth in SEQ ID NO: 34. The present invention also provides the nucleic acid encoding a Rep 52 protein, and in particular an isolated nucleic acid comprising the nucleotide sequence set forth in SEQ ID NO: 32, an isolated nucleic acid consisting essentially of the nucleotide sequence set forth in SEQ ID NO: 32, and a nucleic acid encoding the adeno-associated virus 5 Rep protein having the amino acid sequence set forth in SEQ ID NO: 24. The present invention further provides the nucleic acid encoding a Rep 68 protein and, in particular an isolated nucleic acid comprising the nucleotide sequence set forth in SEQ ID NO: 37, an isolated nucleic acid consisting essentially of the nucleotide sequence set forth in SEQ ID NO: 37, and a nucleic acid encoding the adeno-associated virus 5 protein having the amino acid sequence set forth in SEQ ID NO: 36. And, further, provided is the nucleic acid encoding a Rep 78 protein, and in particular an isolated nucleic acid comprising the nucleotide sequence set forth in SEQ ID NO: 33, an isolated nucleic acid consisting essentially of the nucleotide sequence set forth in SEQ ID NO: 33, and a nucleic acid encoding the adeno-associated virus 5 Rep protein having the amino acid sequence set forth in SEQ ID NO: 25. As described elsewhere herein, these nucleic acids can have minor modifications, including silent nucleotide substitutions, mutations causing conservative amino acid substitutions in the encoded proteins, and mutations in control regions that do not or minimally affect the encoded amino acid sequence.

The present invention further provides a nucleic acid encoding the entire AAV5 Capsid polypeptide. Furthermore, provided is a nucleic acid encoding each of the three AAV5 coat proteins, VP1, VP2, and VP3. Thus, provided is a nucleic acid encoding AAV5 VP1, a nucleic acid encoding AAV5 VP2, and a nucleic acid encoding AAV5 VP3. Thus, provided is a nucleic acid encoding the amino acid sequence set forth in SEQ ID NO: 26 (VP1); a nucleic acid encoding the amino acid sequence set forth in SEQ ID NO: 27(VP2), and a nucleic acid encoding the amino acid sequence set forth in SEQ ID NO: 28 (VP3). The present invention also specifically provides a nucleic acid comprising SEQ ID NO: 29 (VP1 gene); a nucleic acid comprising SEQ ID NO: 30 (VP2 gene); and a nucleic acid comprising SEQ ID NO: 31 (VP3 gene). The present invention also specifically provides a nucleic acid consisting essentially of SEQ ID NO: 29 (VP1 gene), a nucleic acid consisting essentially of SEQ ID NO: 30 (VP2 gene), and a nucleic acid consisting essentially of SEQ ID NO: 31 (VP3 gene). Minor modifications in the nucleotide sequences encoding the capsid, or coat, proteins are contemplated, as described above for other AAV5 nucleic acids. However, in general, a modified nucleic acid encoding a capsid protein will have at least about 85%, about 90%, about 93%, about 95%, about 98% or 100% homology to the capsid nucleic sequences described herein e.g., SEQ ID NOS: 29, 30 and 31, and the capsid polypeptide encoded therein will have overall about 93%, about 95%, about 98%, about 99% or 100% homology with the amino acid sequence described herein, e.g., SEQ ID NOS: 26, 27, and 28. Nucleic acids that selectively hybridize with the nucleic acids of SEQ ID NOS: 29, 30, and 31 under the conditions described above are also provided.

Provided is an isolated AAV5 Rep protein. An AAV5 Rep polypeptide is encoded by ORF1 of AAV5. The present invention also provides each individual AAV5 Rep protein. Thus provided is AAV5 Rep 40 (e.g., SEQ ID NO: 34), or a unique fragment thereof. Provided is AAV5 Rep 52 (e.g., SEQ ID NO: 24), or a unique fragment thereof. Provided is AAV5 Rep 68 (e.g., SEQ ID NO: 36), or a unique fragment thereof. Provided is an example of AAV5 Rep 78 (e.g., SEQ ID NO: 25), or a unique fragment thereof. By “unique fragment thereof” is meant any smaller polypeptide fragment encoded by an AAV5 rep gene that is of sufficient length to be found only in the Rep polypeptide. Substitutions and modifications of the amino acid sequence can be made as described above and, further, can include protein processing modifications, such as glycosylation, to the polypeptide.

The present invention further provides an AAV5 Capsid polypeptide or a unique fragment thereof. AAV5 capsid polypeptide is encoded by ORF 2 of AAV5. The present invention further provides the individual AAV5 capsid proteins, VP1, VP2 and VP3 or unique fragments thereof. Thus, provided is an isolated polypeptide having the amino acid sequence set forth in SEQ ID NO: 26 (VP1). The present invention additionally provides an isolated polypeptide having the amino acid sequence set forth in SEQ ID NO: 27 (VP2). The present invention also provides an isolated polypeptide having the amino acid sequence set forth in SEQ ID NO: 28 (VP3). By “unique fragment thereof” is meant any smaller polypeptide fragment encoded by any AAV5 capsid gene that is of sufficient length to be found only in the AAV5 capsid protein. Substitutions and modifications of the amino acid sequence can be made as described above and, further, can include protein processing modifications, such as glycosylation, to the polypeptide. However, an AAV5 Capsid polypeptide including all three coat proteins will have greater than about 56% overall homology to the polypeptide encoded by the nucleotides set forth in SEQ ID NOS: 26, 27, or 28. The protein can have about 65%, about 70%, about 75%, about 80%, about 85%, about 90%, 93%, 95%, 97% or even 100% homology to the amino acid sequence encoded by the nucleotides set forth in SEQ ID NOS: 26, 27 or 28. An AAV5 VPI polypeptide can have at least about 58%, about 60%, about 70%, about 80%, about 90%, 93%, 95%, 97% or about 100% homology to the amino acid sequence set forth in SEQ ID NO: 26. An AAV5 VP2 polypeptide can have at least about 58%, about 60%, about 70%, about 80%, about 90%, 93%, 95%, 97% or about 100% homology to the amino acid sequence set forth in SEQ ID NO: 27. An AAV5 VP3 polypeptide can have at least about 60%, about 70%, about 80%, about 90%, 93%, 95%, 97% or about 100% homology to the amino acid sequence set forth in SEQ ID NO: 28.

The AAV ITRs in the vector for the herein described delivery methods can be AAV5 ITRs (SEQ ID NOS: 41 and 42). Furthermore, the AAV ITRs in the vector for the herein described nucleic acid delivery methods can also comprise AAV1, AAV2, AAV3, AAV4, or AAV6 inverted terminal repeats.

### Compositions and Methods for Making BAAV Vectors

Compositions and methods for making and using BAAV vectors have been previously described in U.S. Patent Application No. 60/526,786, filed Dec. 4, 2003, and in International Patent Application No. PCT/US04/40825, filed Dec. 6, 2004, which are hereby incorporated herein by reference for this teaching.

Provided is a recombinant bovine adeno-associated virus (BAAV). This virus has one or more of the characteristics described below. The compositions of the present invention do not include wild-type BAAV. The methods of the present invention can use either wild-type BAAV or recombinant BAAV-based delivery.

Provided are novel BAAV particles, recombinant BAAV vectors and recombinant BAAV virions. An BAAV particle is a viral particle comprising an BAAV capsid protein. A recombinant BAAV vector is a nucleic acid construct that comprises at least one unique nucleic acid of BAAV. A recombinant BAAV virion is a particle containing a recombinant BAAV vector, wherin the particle can be either an BAAV particle as described herein or a non-BAAV particle. Alternatively, the recombinant BAAV virion is an BAAV particle containing a recombinant vector, wherein the vector can be either an BAAV vector as described herein or a non-BAAV vector. These vectors, particles, virions, nucleic acids and polypeptides are described below.

Provided is the nucleotide sequence of the BAAV genome and vectors and particles derived therefrom. Specifically, provided is a nucleic acid vector comprising a pair of BAAV inverted terminal repeats (ITRs) and a promoter between the inverted terminal repeats. The rep proteins of AAV5 and BAAV will bind to the BAAV ITR and it can function as an origin of replication for packaging of recombinant AAV particles. The minimum sequence necessary for this activity is the TRS site where Rep cleaves in order to replicate the virus. Minor modifications in an ITR are contemplated and are those that will not interfere with the hairpin structure formed by the ITR as described herein and known in the art. Furthermore, to be considered within the term e.g. it must retain the Rep binding site described herein.

The D− region of the AAV2 ITR, a single stranded region of the ITR, inboard of the TRS site, has been shown to bind a factor which depending on its phosphorylation state correlates with the conversion of the AAV from a single stranded genome to a transcriptionally active form that allows for expression of the viral DNA. This region is conserved between AAV2, 3, 4,and 6 but is divergent in AAV5 and BAAV (SEQ ID NO: 59). The D+ region is the reverse complement of the D− region.

The promoter can be any desired promoter, selected by known considerations, such as the level of expression of a nucleic acid functionally linked to the promoter and the cell type in which the vector is to be used. That is, the promoter can be tissue/cell-specific. Promoters can be prokaryotic, eukaryotic, fungal, nuclear, mitochondrial, viral or plant promoters. Promoters can be exogenous or endogenous to the cell type being transduced by the vector. Promoters can include, for example, bacterial promoters, known strong promoters such as SV40 or the inducible metallothionein promoter, or an AAV promoter, such as an AAV p5 promoter. Additionally, chimeric regulatory promoters for targeted gene expression can be utilized. Examples of these regulatory systems, which are known in the art, include the tetracycline based regulatory system which utilizes the tet transactivator protein (tTA), a chimeric protein containing the VP16 activation domain fused to the tet repressor of Escherichia coli, the IPTG based regulatory system, the CID based regulatory system, and the Ecdysone based regulatory system. Other promoters include promoters derived from actin genes, immunoglobulin genes, cytomegalovirus (CMV), adenovirus, bovine papilloma virus, adenoviral promoters, such as the adenoviral major late promoter, an inducible heat shock promoter, respiratory syncytial virus, Rous sarcomas virus (RSV), etc., specifically, the promoter can be AAV2 p5 promoter or AAV5 p5 promoter or BAAV p5 promoter. More specifically, the BAAV p5 promoter can be in about the same location in SEQ ID NO: 47 as the AAV2 p5 promoter, in the corresponding AAV2 published sequence. Additionally, the p5 promoter may be enhanced by nucleotides 1-173 of SEQ ID NO: 47. Furthermore, smaller fragments of p5 promoter that retain promoter activity can readily be determined by standard procedures including, for example, constructing a series of deletions in the p5 promoter, linking the deletion to a reporter gene, and determining whether the reporter gene is expressed, i.e., transcribed and/or translated. The promoter can be the promoter of any of the AAV serotypes, and can be the p19 promoter (SEQ ID NO: 62) or the p40 promoter set forth in the sequence listing as SEQ ID NO: 63.

It should be recognized that any errors in any of the nucleotide sequences disclosed herein can be corrected, for example, by using the hybridization procedure described below with various probes derived from the described sequences such that the coding sequence can be reisolated and resequenced. Rapid screening for point mutations can also be achieved with the use of polymerase chain reaction single strand conformation polymorphism (PCR SSCP). The corresponding amino acid sequence can then be corrected accordingly.

The BAAV-derived vector can include any normally occurring BAAV nucleic acid sequences in addition to an ITR and promoter. The BAAV-derived vector can also include sequences that are at least 60%, 70%, 80%, 90%, 95%, 96%, 97%, 98%, or 99% identical to the BAAV nucleic acids set forth herein. Examples of vector constructs are provided below.

The present vector or BAAV particle or recombinant BAAV virion can utilize any unique fragment of these present BAAV nucleic acids, including the BAAV nucleic acids set forth in SEQ ID NOS: 47, 48, 50, 52, 54, 56 and 58-63. To be unique, the fragment must be of sufficient size to distinguish it from other known sequences, most readily determined by comparing any nucleic acid fragment to the nucleotide sequences of nucleic acids in computer databases, such as GenBank. Such comparative searches are standard in the art. Typically, a unique fragment useful as a primer or probe will be at least about 8 or 10, preferable at least 20 or 25 nucleotides in length, depending upon the specific nucleotide content of the sequence. Additionally, fragments can be, for example, at least about 30, 40, 50, 75, 100, 200 or 500 nucleotides in length and can encode polypeptides or be probes. The nucleic acid can be single or double stranded, depending upon the purpose for which it is intended. Where desired, the nucleic acid can be RNA.

The present invention further provides a BAAV capsid protein to contain the vector. In particular, provided is not only a polypeptide comprising all three BAAV coat proteins, i.e., VP1, VP2 and VP3, but also a polypeptide comprising each BAAV coat protein individually, SEQ ID NOS: 53, 55, and 57, respectively. Thus, an BAAV particle comprising an BAAV capsid protein comprises at least one BAAV coat protein VP1, VP2 or VP3. A BAAV particle comprising an BAAV capsid protein can be utilized to deliver a nucleic acid vector to a cell, tissue or subject. For example, the herein described BAAV vectors can be encapsidated in an AAV5 capsid-derived particle and utilized in a gene delivery method. Furthermore, other viral nucleic acids can be encapsidated in the BAAV particle and utilized in such delivery methods. For example, an AAV1-8 or AAAV vector (e.g. AAV1-8 or AAAV ITR and nucleic acid of interest) can be encapsidated in an BAAV particle and administered. Furthermore, a BAAV chimeric capsid incorporating both AAV1-8 or AAAV capsid and BAAV capsid sequences can be generated, by standard cloning methods, selecting regions from the known sequences of each protein as desired. For example, particularly antigenic regions of the BAAV capsid protein can be replaced with the corresponding region of the BAAV capsid protein. In addition to chimeric capsids incorporating AAV2 capsid sequences, chimeric capsids incorporating AAV1, 3-8, and AAV5 capsid sequences can be generated, by standard cloning methods, selecting regions from the known sequences of each protein as desired. Alternatively a chimeric capsid can be made by the addition of a plasmid that expresses AAV1-8 capsid proteins at a ratio with the BAAV capsid expression plasmid that allows only a few capsid proteins to be incorporated into the BAAV particle. Thus, for example, a chimeric particle may be constructed that contains 6 AAV2 capsid proteins and 54 BAAV capsid proteins if the complete capsid contains 60 capsid proteins.

The capsids can also be modified to alter their specific tropism by genetically altering the capsid to encode a specific ligand to a cell surface receptor. Alternatively, the capsid can be chemically modified by conjugating a ligand to a cell surface receptor. By genetically or chemically altering the capsids, the tropism can be modified to direct BAAV to a particular cell or population of cells. The capsids can also be altered immunologically by conjugating the capsid to an antibody that recognizes a specific protein on the target cell or population of cells.

It has been recently reported that insertion of foreign epitopes (RGD motif, LH receptor targeting epitope) in certain regions of AAV2 capsid can redirect viral tropism. However, AAV2 naturally infects a wide variety of cell types and complete retargeting of rAAV2 would be difficult to achieve. Provided are two regions in the capsid of BAAV that are on the virus surface and could tolerate substitution. These two regions are aa 257-264 (GSSNASDT, SEQ ID NO:67) and aa 444-457 (TTSGGTLNQGNSAT, SEQ ID NO:68). Other regions of the BAAV capsid could also accommodate the substitution of amino acids that would allow for epitope presentation on the surface of the virus. All of these regions would have in common 1) Surface exposure 2) able to support a substitution of sequence to insert the epitope 3) still allow for capsid assembly.

Because of the symmetry of the AAV particles, a substitution in one subunit of the capsid will appear multiple times on the capsid surface. For example the capsid is made of approximately 55 VP3 proteins. Therefore an epitope incorporated in the VP3 protein could be expressed 55 times on the surface of each particle increasing the likelihood of the epitope forming a stable interaction with its target. In some cases this may be too high of a ligand density for functional binding or this high density of epitope may interfere with capsid formation. The epitope density could be lowered by introducing another plasmid into the packaging system for production of recombinant particles and the ratio between the packaging plasmid with the modified VP3 protein and the wt VP3 protein altered to balance the epitope density on the virus surface.

Epitopes could be incorporated into the virus capsid for the purpose of 1) altering the tropism of the virus 2) blocking an immune response direct at the virus 3) developing a host immune response to the epitope for the purpose of vaccination.

Examples of epitopes that could be added to BAAV capsids include but are not limited to:

LH receptor binding epitope

RGD integrin binding epitope

CD13 binding epitope NGRAHA SEQ ID NO:69

The Retanef polyprotein vaccine candidate for HIV-1

single chain antibody fragments directed against tumor cells

Endothelial cell binding epitope SIGYPLP SEQ ID NO:70

serpin receptor ligand, KFNKPFVFLI SEQ ID NO:71

protective B-cell epitope hemagglutinin (HA) 91-108 from influenza HA

NDV B-cell immunodominant epitope (IDE) spanning residues 447 to 455

Major immunogenic epitope for parvovirus B19 (NISLDNPLENPSSLFDLVARIK, SEQ ID NO:72) that can elicit protective antibody titers.

The capsids can also be assembled into empty particles by expression in mammalian, bacterial, fungal or insect cells. For example, AAV2 particles are known to be made from VP3 and VP2 capsid proteins in baculovirus. The same basic protocol can produce an empty BAAV particle comprising BAAV capsid proteins and also full particles.

The herein described recombinant BAAV nucleic acid derived vector can be encapsidated in an AAV particle. In particular, it can be encapsidated in an AAV1 particle, an AAV2 particle, an AAV3 particle, an AAV4 particle, an AAVS particle or an AAV6 or AAV7 or an AAV8 or AAAV particle, a portion of any of these capsids, or a chimeric capsid particle as described above, by standard methods using the appropriate capsid proteins in the encapsidation process, as long as the nucleic acid vector fits within the size limitation of the particle utilized. The encapsidation process itself is standard in the art. The BAAV replication machinery, i.e. the rep initiator proteins and other functions required for replication, can be utilized to produce the BAAV genome that can be packaged in an AAV1-8 or AAAV capsid.

The recombinant BAAV virion containing a vector can also be produced by recombinant methods utilizing multiple plasmids. In one example, the BAAV rep nucleic acid would be cloned into one plasmid, the BAAV int nucleic acid would be cloned into another plasmid and the AAV1-8 capsid nucleic acid would be cloned on another plasmid. These plasmids would then be introduced into cells. The cells that were efficiently transduced by all three plasmids, would exhibit specific integration as well as the ability to produce BAAV recombinant virus. Additionally, two plasmids could be used where the BAAV rep nucleic acid would be cloned into one plasmid and the BAAV ITR and BAAV capsid would be cloned into another plasmid. These plasmids would then be introduced into cells. The cells that were efficiently transduced by both plasmids, would exhibit specific integration as well as the ability to produce BAAV recombinant virus.

An BAAV capsid polypeptide encoding the entire VP1, VP2, and VP3 polypeptide can overall have greater than 56% homology to the polypeptide having the amino acid sequence encoded by nucleotides in SEQ ID NOS: 52, 54 and 56. The capsid protein can have about 70% homology, about 75% homology, 80% homology, 85% homology, 90% homology, 95% homology, 98% homology, 99% homology, or even 100% homology to the protein having the amino acid sequence encoded by the nucleotides set forth in SEQ ID NOS: 52, 54 and 56. The percent homology used to identify proteins herein, can be based on a nucleotide-by-nucleotide comparison or more preferable is based on a computerized algorithm as described herein. Variations in the amino acid sequence of the BAAV capsid protein are contemplated herein, as long as the resulting particle comprising an BAAV capsid protein remains antigenically or immunologically distinct from AAV1-8 or AAAV capsid, as can be routinely determined by standard methods. Specifically, for example, ELISA and Western blots can be used to determine whether a viral particle is antigenically or immunologically distinct from AAV2 or the other serotypes. Furthermore, the BAAV particle preferably retains tissue tropism distinction from other AAVs, such as that exemplified in the examples herein. A BAAV chimeric particle comprising at least one BAAV coat protein may have a different tissue tropism from that of an BAAV particle consisting only of BAAV coat proteins, but is still distinct from the tropism of an AAV2 particle.

The invention further provides a recombinant BAAV virion, comprising a BAAV particle containing, i.e., encapsidating, a vector comprising a pair of BAAV inverted terminal repeats. The recombinant vector can further comprise a BAAV Rep-encoding nucleic acid. The vector encapsidated in the particle can further comprise an exogenous nucleic acid inserted between the inverted terminal repeats.

The invention further contemplates chimeric recombinant ITRs that contain a rep binding site and a TRS site recognized by that Rep protein. By “Rep protein” is meant all four of the Rep proteins, Rep 40, Rep 78, Rep 52, Rep 68. Alternatively, “Rep protein” could be one or more of the Rep proteins described herein. One example of a chimeric ITR would consist of an BAAV D region (SEQ ID NO: 59), an BAAV TRS site (SEQ ID NO: 60), an AAV2 hairpin and an AAV2 Rep binding site. Another example would be a BAAV D region, an BAAV TRS site, an AAV3 hairpin and an AAV3 Rep binding site. In these chimeric ITRs, the D region can be from AAV1-8 or AAAV. The hairpin can be derived from AAV 1-8 or AAAV. The binding site can be derived from any of AAV1-8 or AAAV. Preferably, the D region and the TRS are from the same serotype.

The chimeric ITRs can be combined with BAAV Rep protein and any of the AAV serotype capsids to obtain recombinant virion. For example, recombinant virion can be produced by a BAAV D region, an BAAV TRS site, an AAV2 hairpin, an AAV2 binding site, BAAV Rep protein and AAV1 capsid. This recombinant virion would possess the cellular tropism conferred by the AAV1 capsid protein and would possess the efficient replication conferred by the BAAV Rep.

Other examples of the ITR, Rep protein and Capsids that will produce recombinant virus are provided in the list below but not limited to:

BAAV ITR+BAAV Rep+BAAV Cap=virus

AAV5 ITR+BAAV Rep+BAAV Cap=virus

AAV5 ITR+BAAV Rep+AAV1 Cap=virus

AAV5 ITR+BAAV Rep+AAV2 Cap=virus

AAV5 ITR+BAAV Rep+AAV3 Cap=virus

AAV5 ITR+BAAV Rep+AAV4 Cap=virus

AAV5 ITR+BAAV Rep+AAV5 Cap=virus

AAV5 ITR+BAAV Rep+AAV6 Cap=virus

AAV5 ITR+BAAV Rep+AAV7 Cap=virus

AAV5 ITR+BAAV Rep+AAV8 Cap=virus

BAAV ITR+AAV5 Rep+BAAV Cap=virus

BAAV ITR+AAV5 Rep+AAV1 Cap=virus

BAAV ITR+AAV5 Rep+AAV2 Cap=virus

BAAV ITR+AAV5 Rep+AAV3 Cap=virus

BAAV ITR+AAV5 Rep+AAV4 Cap=virus

BAAV ITR+AAV5 Rep+AAV5 Cap=virus

BAAV ITR+AAV5 Rep+AAV6 Cap=virus

BAAV ITR+AAV5 Rep+AAV7 Cap=virus

BAAV ITR+AAV5 Rep+AAV8 Cap=virus

AAV5 ITR+AAV5 Rep+BAAV Cap=virus

AAV1 ITR+AAV1 Rep+BAAV Cap=virus

AAV2 ITR+AAV2 Rep+BAAV Cap=virus

AAV3 ITR+AAV3 Rep+BAAV Cap=virus

AAV4 ITR+AAV4 Rep+BAAV Cap=virus

AAV5 ITR+AAV5 Rep+BAAV Cap=virus

AAV6 ITR+AAV6 Rep+BAAV Cap=virus

AAV7 ITR+AAV7 Rep+BAAV Cap=virus

AAV8 ITR+AAV8 Rep+BAAV Cap=virus

In any of the constructs described herein, inclusion of a promoter is preferred. As used in the constructs herein, unless otherwise specified, Cap (capsid) refers to any of BAAV VP1, BAAV VP2, BAAV VP3, combinations thereof, functional fragments of any of VP1, VP2 or VP3, or chimeric capsids as described herein. The ITRs of the constructs described herein, can be chimeric recombinant ITRs as described elsewhere in the application.

Conjugates of recombinant or wild-type BAAV virions and nucleic acids or proteins can be used to deliver those molecules to a cell. For example, the purified BAAV can be used as a vehicle for delivering DNA bound to the exterior of the virus. Examples of this are to conjugate the DNA to the virion by a bridge using poly L lysine or other charged molecule. Also contemplated are virosomes that contain BAAV structural proteins (BAAV capsid proteins), lipids such as DOTAP, and nucleic acids that are complexed via charge interaction to introduce DNA into cells.

Also provided by this invention are conjugates that utilize the BAAV capsid or a unique region of the BAAV capsid protein (e.g. VP1, VP2 or VP3 or combinations thereof) to introduce DNA into cells. For example, the BAAV VP3 protein or fragment thereof, can be conjugated to a DNA on a plasmid that is conjugated to a lipid. Cells can be infected using the targeting ability of the VP3 capsid protein to achieve the desired tissue tropism, specific to BAAV. BAAV VP1 and VP2 proteins can also be utilized to introduce DNA or other molecules into cells. By further incorporating the Rep protein and the AAV TRS into the DNA-containing conjugate, cells can be transduced and targeted integration can be achieved. For example, if BAAV specific targeted integration is desired, a conjugate composed of the BAAV VP3 capsid, BAAV rep or a fragment of BAAV rep, BAAV TRS, the rep binding site, the heterologous DNA of interest, and a lipid, can be utilized to achieve BAAV specific tropism and BAAV specific targeted integration in the genome.

Further provided by this invention are chimeric viruses where BAAV can be combined with herpes virus, baculovirus or other viruses to achieve a desired tropism associated with another virus. For example, the BAAV ITRs could be inserted in the herpes virus and cells could be infected. Post-infection, the ITRs of BAAV could be acted on by BAAV rep provided in the system or in a separate vehicle to rescue BAAV from the genome. Therefore, the cellular tropism of the herpes simplex virus can be combined with BAAV rep mediated targeted integration. Other viruses that could be utilized to construct chimeric viruses include lentivirus, retrovirus, pseudotyped retroviral vectors, and adenoviral vectors.

The present invention further provides isolated nucleic acids of BAAV. For example, provided is an isolated nucleic acid comprising the nucleotide sequence set forth in SEQ ID NO: 47 (BAAV genome). This nucleic acid, or portions thereof, can be inserted into vectors, such as plasmids, yeast artificial chromosomes, or other viral vector (particle), if desired, by standard cloning methods. The present invention also provides an isolated nucleic acid consisting essentially of the nucleotide sequence set forth in SEQ ID NO: 47. The nucleotides of SEQ ID NO: 47 can have minor modifications and still be contemplated by the present invention. For example, modifications that do not alter the amino acid encoded by any given codon (such as by modification of the third, “wobble,” position in a codon) can readily be made, and such alterations are known in the art. Furthermore, modifications that cause a resulting neutral (conserved) amino acid substitution of a similar amino acid can be made in a coding region of the genome. Additionally, modifications as described herein for the BAAV components, such as the ITRs, the p5 promoter, etc. are contemplated in this invention. Furthermore, modifications to regions of SEQ ID NO:4 47 other than in the ITR, TRS, Rep binding site and hairpin are likely to be tolerated without serious impact on the function of the nucleic acid as a recombinant vector.

The present invention additionally provides an isolated nucleic acid that selectively hybridizes with any nucleic acid disclosed herein, including the entire BAAV genome and any unique fragment thereof, including the Rep and capsid encoding sequences (e.g. SEQ ID NOS: 47, 48, 50, 52, 54, 56, 58, 59, 60, 61, 62, 63). Specifically, the nucleic acid can selectively or specifically hybridize to an isolated nucleic acid consisting of the nucleotide sequence set forth in SEQ ID NO: 47 (BAAV genome). The present invention further provides an isolated nucleic acid that selectively or specifically hybridizes with an isolated nucleic acid comprising the nucleotide sequence set forth in SEQ ID NO: 47 (BAAV genome). By “selectively hybridizes” as used herein is meant a nucleic acid that hybridizes to one of the disclosed nucleic acids under sufficient stringency conditions without significant hybridization to a nucleic acid encoding an unrelated protein, and particularly, without detectably hybridizing to nucleic acids of AAV2. Thus, a nucleic acid that selectively hybridizes with a nucleic acid of the present invention will not selectively hybridize under stringent conditions with a nucleic acid encoding a different protein or the corresponding protein from a different serotype of the virus, and vice versa. A “specifically hybridizing” nucleic acid is one that hybridizes under stringent conditions to only a nucleic acid found in BAAV. Therefore, nucleic acids for use, for example, as primers and probes to detect or amplify the target nucleic acids are contemplated herein. Nucleic acid fragments that selectively hybridize to any given nucleic acid can be used, e.g., as primers and or probes for further hybridization or for amplification methods (e.g., polymerase chain reaction (PCR), ligase chain reaction (LCR)). Additionally, for example, a primer or probe can be designed that selectively hybridizes with both BAAV and a gene of interest carried within the BAAV vector (i.e., a chimeric nucleic acid).

A nucleic acid that selectively hybridizes to any portion of the BAAV genome is contemplated herein. Therefore, a nucleic acid that selectively hybridizes to BAAV can be of longer length than the BAAV genome, it can be about the same length as the BAAV genome or it can be shorter than the BAAV genome. The length of the nucleic acid is limited on the shorter end of the size range only by its specificity for hybridization to BAAV, i.e., once it is too short, typically less than about 5 to 7 nucleotides in length, it will no longer bind specifically to BAAV, but rather will hybridize to numerous background nucleic acids. Additionally contemplated by this invention is a nucleic acid that has a portion that specifically hybridizes to BAAV and a portion that specifically hybridizes to a gene of interest inserted within BAAV.

The present invention further provides an isolated nucleic acid encoding a bovine adeno-associated virus Rep protein. The BAAV Rep proteins are encoded by open reading frame (ORF) 1 of the BAAV genome. Examples of the BAAV Rep genes are shown in the nucleic acid set forth in SEQ ID NO: 47, and include nucleic acids consisting essentially of the nucleotide sequences set forth in SEQ ID NOS: 48 (rep78), 4(rep52) and nucleic acids comprising the nucleotide sequences set forth in SEQ ID NOS: 48 and 50. However, the present invention contemplates that the Rep nucleic acid can include any one, two, three, or four of the four Rep proteins, in any order, in such a nucleic acid. Furthermore, minor modifications are contemplated in the nucleic acid, such as silent mutations in the coding sequences, mutations that make neutral or conservative changes in the encoded amino acid sequence, and mutations in regulatory regions that do not disrupt the expression of the gene. Examples of other minor modifications are known in the art. Further modifications can be made in the nucleic acid, such as to disrupt or alter expression of one or more of the Rep proteins in order to, for example, determine the effect of such a disruption; such as to mutate one or more of the Rep proteins to determine the resulting effect, etc. However, in general, a modified nucleic acid encoding a Rep protein will have at least about 85%, about 90%, about 93%, about 95%, about 98% or 100% homology to the Rep nucleic sequences described herein e.g., SEQ ID NOS: 48 and 50, and the Rep polypeptide encoded therein will have overall about 93%, about 95%, about 98%, about 99% or 100% homology with the amino acid sequence described herein, e.g., SEQ ID NOS: 49 and 51. Percent homology is determined by the techniques described herein.

The present invention also provides an isolated nucleic acid that selectively or specifically hybridizes with a nucleic acid consisting essentially of the nucleotide sequence set forth in SEQ ID NOS: 48 and 50, and an isolated nucleic acid that selectively hybridizes with a nucleic acid comprising the nucleotide sequence set forth in SEQ ID NOS: 48 and 50. “Selectively hybridizing” and “stringency of hybridization” is defined elsewhere herein.

As described above, provided is the nucleic acid encoding a Rep 78 protein and, in particular an isolated nucleic acid comprising the nucleotide sequence set forth in SEQ ID NO: 48, an isolated nucleic acid consisting essentially of the nucleotide sequence set forth in SEQ ID NO: 48, and a nucleic acid encoding the bovine adeno-associated virus protein having the amino acid sequence set forth in SEQ ID NO: 49. The present invention also provides the nucleic acid encoding a Rep 52 protein, and in particular an isolated nucleic acid comprising the nucleotide sequence set forth in SEQ ID NO: 50, an isolated nucleic acid consisting essentially of the nucleotide sequence set forth in SEQ ID NO: 50, and a nucleic acid encoding the bovine adeno-associated virus Rep 52 protein having the amino acid sequence set forth in SEQ ID NO: 51. As described elsewhere herein, these nucleic acids can have minor modifications, including silent nucleotide substitutions, mutations causing conservative amino acid substitutions in the encoded proteins, and mutations in control regions that do not or minimally affect the encoded amino acid sequence.

The present invention further provides a nucleic acid encoding the entire BAAV Capsid polypeptide. Furthermore, provided is a nucleic acid encoding each of the three BAAV coat proteins, VP1, VP2, and VP3. Thus, provided is a nucleic acid encoding BAAV VP1, a nucleic acid encoding BAAV VP2, and a nucleic acid encoding BAAV VP3. Thus, provided is a nucleic acid encoding the amino acid sequence set forth in SEQ ID NO: 53 (VP1); a nucleic acid encoding the amino acid sequence set forth in SEQ ID NO: 55 (VP2), and a nucleic acid encoding the amino acid sequence set forth in SEQ ID NO: 57 (VP3). The present invention also specifically provides a nucleic acid comprising SEQ ID NO: 52 (VP1 gene); a nucleic acid comprising SEQ ID NO: 54 (VP2 gene); and a nucleic acid comprising SEQ ID NO: 56 (VP3 gene). The present invention also specifically provides a nucleic acid consisting essentially of SEQ ID NO: 52 (VP1 gene), a nucleic acid consisting essentially of SEQ ID NO: 54 (VP2 gene), and a nucleic acid consisting essentially of SEQ ID NO: 56 (VP3 gene). Minor modifications in the nucleotide sequences encoding the capsid, or coat, proteins are contemplated, as described above for other BAAV nucleic acids. However, in general, a modified nucleic acid encoding a capsid protein will have at least about 85%, about 90%, about 93%, about 95%, about 98% or 100% homology to the capsid nucleic sequences described herein e.g., SEQ ID NOS: 52, 54 and 56, and the capsid polypeptide encoded therein will have overall about 93%, about 95%, about 98%, about 99% or 100% homology with the amino acid sequence described herein, e.g., SEQ ID NOS: 53, 55 and 57. Nucleic acids that selectively hybridize with the nucleic acids of SEQ ID NOS: 52, 54 and 56 under the conditions described above are also provided.

Provided is an isolated BAAV Rep protein. An BAAV Rep polypeptide is encoded by ORF1 of BAAV. The present invention also provides each individual BAAV Rep protein. Thus provided is BAAV Rep 52 (e.g., SEQ ID NO: 50), or a unique fragment thereof. Provided is BAAV Rep 78 (e.g., SEQ ID NO: 48), or a unique fragment thereof. By “unique fragment thereof” is meant any smaller polypeptide fragment encoded by an BAAV rep gene that is of sufficient length to be found only in the Rep polypeptide. Substitutions and modifications of the amino acid sequence can be made as described above and, further, can include protein processing modifications, such as glycosylation, to the polypeptide.

The present invention further provides a BAAV Capsid polypeptide or a unique fragment thereof. BAAV capsid polypeptide is encoded by ORF 2 of BAAV. The present invention further provides the individual BAAV capsid proteins, VP1, VP2 and VP3 or unique fragments thereof. Thus, provided is an isolated polypeptide having the amino acid sequence set forth in SEQ ID NO:52 (VP1). The present invention additionally provides an isolated polypeptide having the amino acid sequence set forth in SEQ ID NO: 54 (VP2). The present invention also provides an isolated polypeptide having the amino acid sequence set forth in SEQ ID NO:56 (VP3). By “unique fragment thereof” is meant any smaller polypeptide fragment encoded by any BAAV capsid gene that is of sufficient length to be found only in the BAAV capsid protein. Substitutions and modifications of the amino acid sequence can be made as described above and, further, can include protein processing modifications, such as glycosylation, to the polypeptide. However, an BAAV Capsid polypeptide including all three coat proteins will have greater than about 56% overall homology to the polypeptide encoded by the nucleotides set forth in SEQ ID NOS: 52, 54 or 56. The protein can have about 65%, about 70%, about 75%, about 80%, about 85%, about 90%, 93%, 95%, 97% or even 100% homology to the amino acid sequence encoded by the nucleotides set forth in SEQ ID NOS: 52, 54 or 56. An BAAV VP1 polypeptide can have at least about 58%, about 60%, about 70%, about 80%, about 90%, 93%, 95%, 97% or about 100% homology to the amino acid sequence set forth in SEQ ID NO: 53. An BAAV VP2 polypeptide can have at least about 58%, about 60%, about 70%, about 80%, about 90%, 93%, 95%, 97% or about 100% homology to the amino acid sequence set forth in SEQ ID NO: 55. An BAAV VP3 polypeptide can have at least about 60%, about 70%, about 80%, about 90%, 93%, 95%, 97% or about 100% homology to the amino acid sequence set forth in SEQ ID NO: 57.

The present invention also provides a method of producing the BAAV virus by transducing a cell with the nucleic acid encoding the virus.

The present method further provides a method of delivering an exogenous (heterologous) nucleic acid to a cell comprising administering to the cell an BAAV particle containing a vector comprising the nucleic acid inserted between a pair of AAV inverted terminal repeats, thereby delivering the nucleic acid to the cell.

The AAV ITRs in the vector for the herein described delivery methods can be AAV ITRs (SEQ ID NOS: 58). Furthermore, the AAV ITRs in the vector for the herein described nucleic acid delivery methods can also comprise AAV1-8 or AAAV inverted terminal repeats.

### Compositions and Methods for Making AAV7 Vectors

Compositions and methods for making and using AAV7 vectors have been previously described in Gao GP, et al. Proc Natl Acad Sci USA. 2002 Sep. 3; 99(18):11854-9; U.S. Patent Application 2003/0228282; and International Patent Application No. PCT/US02/33630, which are hereby incorporated by reference herein for the teaching of compositions and method for making and using AAV7 virions, vectors, and particles. Provided is a recombinant adeno-associated virus-7 (AAV7). This virus has one or more of the characteristics described below. The compositions of the present invention do not include wild-type AAV7. The methods of the present invention can use either wild-type AAV7 or recombinant AAV7-based delivery.

Provided are AAV7 particles, recombinant AAV7 vectors and recombinant AAV7 virions. An AAV7 particle is a viral particle comprising an AAV7 capsid protein. A recombinant AAV7 vector is a nucleic acid construct that comprises at least one unique nucleic acid of AAV7. A recombinant AAV7 virion is a particle containing a recombinant AAV7 vector, wherein the particle can be either an AAV7 particle as described herein or a non-AAV7 particle. Alternatively, the recombinant AAV7 virion is an AAV7 particle containing a recombinant vector, wherein the vector can be either an AAV7 vector as described herein or a non-AAV7 vector. These vectors, particles, virions, nucleic acids and polypeptides are described below.

The AAV7-derived vector can include any normally occurring AAV7 nucleic acid sequences. The AAV7-derived vector can also include sequences that are at least 60%, 70%, 80%, 90%, 95%, 96%, 97%, 98%, or 99% identical to the AAV7 nucleic acids set forth herein. Examples of vector constructs are provided below.

The present vector or AAV7 particle or recombinant AAV7 virion can utilize any unique fragment of the present AAV7 nucleic acids, including the AAV7 nucleic acids set forth in SEQ ID NO:64. Fragments can be, for example, at least about 30, 40, 50, 75, 100, 200 or 500 nucleotides in length. The nucleic acid can be single or double stranded, depending upon the purpose for which it is intended.

The present invention further provides an AAV7 capsid protein to contain the vector. In particular, provided is a polypeptide comprising AAV7 capsid protein, SEQ ID NO:66. An AAV7 particle comprising an AAV7 capsid protein can be utilized to deliver a nucleic acid vector to a cell, tissue or subject. For example, the herein described AAV7 vectors can be encapsidated in an AAV5 capsid-derived particle and utilized in a gene delivery method. Furthermore, other viral nucleic acids can be encapsidated in the AAV7 particle and utilized in such delivery methods. For example, an AAV1-6, 8, BAAV or AAAV vector (e.g. AAV1-6, 8, BAAV or AAAV ITR and nucleic acid of interest) can be encapsidated in an AAV7 particle and administered. Furthermore, a AAV7 chimeric capsid incorporating AAV1-6, 8, BAAV or AAAV capsid, and AAV7 capsid sequences can be generated, by standard cloning methods, selecting regions from the known sequences of each protein as desired. For example, particularly antigenic regions of the AAV2 capsid protein can be replaced with the corresponding region of the AAV7 capsid protein. In addition to chimeric capsids incorporating AAV2 capsid sequences, chimeric capsids incorporating AAV1, 3-6, 8, BAAV and AAV5 capsid sequences can be generated, by standard cloning methods, selecting regions from the known sequences of each protein as desired. Alternatively a chimeric capsid can be made by the addition of a plasmid that expresses AAV1, 3-6, 8, BAAV or AAV5 capsid proteins at a ratio with the AAV7 capsid expression plasmid that allows only a few capsid proteins to be incorporated into the AAV7 particle. Thus, for example, a chimeric particle may be constructed that contains 6 AAV2 capsid proteins and 54 AAV7 capsid proteins if the complete capsid contains 60 capsid proteins.

The capsids can also be assembled into empty particles by expression in mammalian, bacterial, fungal or insect cells. For example, AAV2 particles are known to be made from VP3 and VP2 capsid proteins in baculovirus. The same basic protocol can produce an empty AAV7 particle comprising AAV7 capsid proteins and also full particles.

The herein described recombinant AAV7 nucleic acid derived vector can be encapsidated in an AAV particle. In particular, it can be encapsidated in an AAV1 particle, an AAV2 particle, an AAV3 particle, an AAV4 particle, an AAV5 particle, an AAV6, an AAV8, a BAAV particle or AAAV particle, a portion of any of these capsids, or a chimeric capsid particle as described above, by standard methods using the appropriate capsid proteins in the encapsidation process, as long as the nucleic acid vector fits within the size limitation of the particle utilized. The encapsidation process itself is standard in the art. The AAV7 replication machinery, i.e. the rep initiator proteins and other functions required for replication, can be utilized to produce the AAV7 genome that can be packaged in an AAV1-6, 8, BAAV or AAAV capsid.

The recombinant AAV7 virion containing a vector can also be produced by recombinant methods utilizing multiple plasmids. In one example, the AAV7 rep nucleic acid would be cloned into one plasmid, the AAV2 ITR nucleic acid would be cloned into another plasmid and the AAV7 capsid nucleic acid would be cloned on another plasmid. These plasmids would then be introduced into cells. The cells that were efficiently transduced by all three plasmids, would exhibit specific integration as well as the ability to produce AAV7 recombinant virus. Additionally, two plasmids could be used where the AAV7 rep nucleic acid would be cloned into one plasmid and the AAV7 ITR and AAV7 capsid would be cloned into another plasmid. These plasmids would then be introduced into cells. The cells that were efficiently transduced by both plasmids, would exhibit specific integration as well as the ability to produce AAV7 recombinant virus.

An AAV7 capsid polypeptide encoding the entire VP1 polypeptide can overall have greater than 56% homology to the polypeptide having the amino acid sequence encoded by nucleotides in SEQ ID NO:66. The capsid protein can have about 70% homology, about 75% homology, 80% homology, 85% homology, 90% homology, 95% homology, 98% homology, 99% homology, or even 100% homology to the protein having the amino acid sequence encoded by the nucleotides set forth in SEQ ID NO:66. The percent homology used to identify proteins herein, can be based on a nucleotide-by-nucleotide comparison or more preferable is based on a computerized algorithm as described herein. Variations in the amino acid sequence of the AAV7 capsid protein are contemplated herein, as long as the resulting particle comprising an AAV7 capsid protein remains antigenically or immunologically distinct from AAV1-6, 8, BAAV or AAAV capsid, as can be routinely determined by standard methods. Specifically, for example, ELISA and Western blots can be used to determine whether a viral particle is antigenically or immunologically distinct from AAV2 or the other serotypes. Furthermore, the AAV7 particle preferably retains tissue tropism distinction from other AAVs. An AAV7 chimeric particle comprising at least one AAV7 coat protein may have a different tissue tropism from that of an AAV7 particle consisting only of AAV7 coat proteins, but is still distinct from the tropism of an AAV2 particle.

The invention further provides a recombinant AAV7 virion, comprising an AAV7 particle containing, i.e., encapsidating, a vector comprising a pair of AAV7 inverted terminal repeats. The recombinant vector can further comprise an AAV7 Rep-encoding nucleic acid. The vector encapsidated in the particle can further comprise an exogenous nucleic acid inserted between the inverted terminal repeats.

For example, recombinant virion can be produced by a AAV2 ITR, AAV2 Rep protein and AAV7 capsid. This recombinant virion would possess the cellular tropism conferred by the AAV7 capsid protein and would possess the efficient replication conferred by the AAV2 Rep.

Other examples of the ITR, Rep protein and Capsids that will produce recombinant virus are provided in the list below but not limited to:

AAV5 ITR+AAV7 Rep+AAV1 Cap=virus

AAV5 ITR+AAV7 Rep+AAV2 Cap=virus

AAV5 ITR+AAV7 Rep+AAV3 Cap=virus

AAV5 ITR+AAV7 Rep+AAV4 Cap=virus

AAV5 ITR+AAV7 Rep+AAV5 Cap=virus

AAV5 ITR+AAV7 Rep+AAV6 Cap=virus

AAV5 ITR+AAV7 Rep+AAV7 Cap=virus

AAV5 ITR+AAV7 Rep+AAV8 Cap=virus

AAV5 ITR+AAV7 Rep+BAAV Cap=virus

AAV5 ITR+AAV7 Rep+AAAV Cap=virus

AAV1 ITR+AAV1 Rep+AAV7 Cap=virus

AAV2 ITR+AAV2 Rep+AAV7 Cap=virus

AAV3 ITR+AAV3 Rep+AAV7 Cap=virus

AAV4 ITR+AAV4 Rep+AAV7 Cap=virus

AAV5 ITR+AAV5 Rep+AAV7 Cap=virus

AAV6 ITR+AAV6 Rep+AAV7 Cap=virus

AAV8 ITR+AAV8 Rep+AAV7 Cap=virus

BAAV+BAAV Rep+AAV7 Cap=virus

AAAV ITR+AAAV Rep+AAV7 Cap=virus

In any of the constructs described herein, inclusion of a promoter is preferred. As used in the constructs herein, unless otherwise specified, Cap (capsid) refers to any of AAV7 VP1, AAV7 VP2, AAV7 VP3, combinations thereof, functional fragments of any of VP1, VP2 or VP3, or chimeric capsids as described herein. The ITRs of the constructs described herein, can be chimeric recombinant ITRs as described elsewhere in the application.

Conjugates of recombinant or wild-type AAV7 virions and nucleic acids or proteins can be used to deliver those molecules to a cell. For example, the purified AAV7 can be used as a vehicle for delivering DNA bound to the exterior of the virus. Examples of this are to conjugate the DNA to the virion by a bridge using poly L lysine or other charged molecule. Also contemplated are virosomes that contain AAV7 structural proteins (AAV7 capsid proteins), lipids such as DOTAP, and nucleic acids that are complexed via charge interaction to introduce DNA into cells.

Also provided by this invention are conjugates that utilize the AAV7 capsid or a unique region of the AAV7 capsid protein (e.g. VP1, VP2 or VP3 or combinations thereof) to introduce DNA into cells. By “unique” is meant any smaller polypeptide fragment encoded by any AAV7 capsid gene that is of sufficient length to be unique to the AAV7 Capsid protein. For example, the AAV7 VP1 protein or fragment thereof, can be conjugated to a DNA on a plasmid that is conjugated to a lipid. Cells can be infected using the targeting ability of the VP1 capsid protein to achieve the desired tissue tropism, specific to AAV7. AAV7 VP1 proteins can also be utilized to introduce DNA or other molecules into cells. By further incorporating an AAV Rep protein and an AAV TRS into the DNA-containing conjugate, cells can be transduced and targeted integration can be achieved.

The present invention further provides isolated nucleic acids of AAV7. For example, provided is an isolated nucleic acid comprising the nucleotide sequence set forth in SEQ ID NO:64. This nucleic acid, or portions thereof, can be inserted into vectors, such as plasmids, yeast artificial chromosomes, or other viral vector (particle), if desired, by standard cloning methods. The present invention also provides an isolated nucleic acid consisting essentially of the nucleotide sequence set forth in SEQ ID NO:64. The nucleotides of SEQ ID NO:64 can have minor modifications and still be contemplated by the present invention. For example, modifications that do not alter the amino acid encoded by any given codon (such as by modification of the third, “wobble,” position in a codon) can readily be made, and such alterations are known in the art. Furthermore, modifications that cause a resulting neutral (conserved) amino acid substitution of a similar amino acid can be made in a coding region of the genome.

The present invention also provides an isolated nucleic acid that selectively or specifically hybridizes with a nucleic acid consisting essentially of the nucleotide sequence set forth in SEQ ID NO:64, and an isolated nucleic acid that selectively hybridizes with a nucleic acid comprising the nucleotide sequence set forth in SEQ ID NO:64. “Selectively hybridizing” and “stringency of hybridization” is defined elsewhere herein.

The present invention further provides an isolated nucleic acid encoding a AAV7 Rep protein. The AAV7 Rep proteins are encoded by open reading frame (ORF) 1 of the AAV7 genome. Examples of the AAV7 Rep genes are shown in the nucleic acid set forth in nucleotides 334-2205 of SEQ ID NO:64, and include nucleic acids consisting essentially of the nucleotide sequences set forth in 334-2205 of SEQ ID NO:64 (rep78). Minor modifications are contemplated in the nucleic acid, such as silent mutations in the coding sequences, mutations that make neutral or conservative changes in the encoded amino acid sequence, and mutations in regulatory regions that do not disrupt the expression of the gene. Examples of other minor modifications are known in the art. Further modifications can be made in the nucleic acid, such as to disrupt or alter expression of one or more of the Rep proteins in order to, for example, determine the effect of such a disruption; such as to mutate one or more of the Rep proteins to determine the resulting effect, etc. However, in general, a modified nucleic acid encoding a Rep protein will have at least about 85%, about 90%, about 93%, about 95%, about 98% or 100% homology to the Rep nucleic sequences described herein e.g., SEQ ID NOS:65, and the Rep polypeptide encoded therein will have overall about 93%, about 95%, about 98%, about 99% or 100% homology with the amino acid sequence described in SEQ ID NO:65. Percent homology is determined by the techniques described herein.

The present invention further provides a nucleic acid encoding the entire AAV7 Capsid polypeptide. Thus, provided is a nucleic acid encoding the amino acid sequence set forth in nucleotides 2222-4435 of SEQ ID NO:64 (VP1). Minor modifications in the nucleotide sequences encoding the capsid, or coat, proteins are contemplated, as described above for other AAV7 nucleic acids. However, in general, a modified nucleic acid encoding a capsid protein will have at least about 85%, about 90%, about 93%, about 95%, about 98% or 100% homology to the capsid nucleic sequences described herein e.g., nucleotides 2222-4435 of SEQ ID NO:64, and the capsid polypeptide encoded therein will have overall about 93%, about 95%, about 98%, about 99% or 100% homology with the amino acid sequence described herein, e.g., SEQ ID NO:66.

### AAV Vector Generation

It is understood that as discussed herein the use of the terms “homology” and “identity” mean the same thing as similarity. Thus, for example, if the use of the word homology is used to refer to two non-natural sequences, it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related.

In general, it is understood that one way to define any known variants and derivatives or those that might arise, of the disclosed nucleic acids and polypeptides herein, is through defining the variants and derivatives in terms of homology to specific known sequences. In general, variants of nucleic acids and polypeptides herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two polypeptides or nucleic acids. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.; the BLAST algorithm of Tatusova and Madden FEMS Microbiol. Lett. 174: 247-250 (1999) available from the National Center for Biotechnology Information (http://www.ncbi.nlm.nih.gov/blast/b12seq/b12.html)), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

Stringency of hybridization is controlled by both temperature and salt concentration of either or both of the hybridization and washing steps. Typically, the stringency of hybridization to achieve selective hybridization involves hybridization in high ionic strength solution (6×SSC or 6× SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The washing temperatures can be used as described above to achieve selective stringency, as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987: 154:367, 1987). A preferable stringent hybridization condition for a DNA:DNA hybridization can be at about 68° C. (in aqueous solution) in 6×SSC or 6× SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

In vivo administration to a human subject or an animal model can be by any, of many standard means for administering viruses, depending upon the target organ, tissue or cell. Virus particles can be administered orally, parenterally (e.g., intravenously), by intramuscular injection, intrarectally, by direct tissue or organ injection, by intraperitoneal injection, topically, transdermally, via aerosol delivery, via the mucosa or the like. Viral nucleic acids (non-encapsidated) can also be administered, e.g., as a complex with cationic liposomes, or encapsulated in anionic liposomes. The present compositions can include various amounts of the selected viral particle or non-encapsidated viral nucleic acid in combination with a pharmaceutically acceptable carrier and, in addition, if desired, may include other medicinal agents, pharmaceutical agents, carriers, adjuvants, diluents, etc. Parental administration, if used, is generally characterized by injection. Injectables can be prepared in conventional forms, either as liquid solutions or suspensions, solid forms suitable for solution or suspension in liquid prior to injection, or as emulsions. Dosages will depend upon the mode of administration, the disease or condition to be treated, and the individual subject's condition, but will be that dosage typical for and used in administration of other AAV vectors, such as AAV2 vectors. Often a single dose can be sufficient; however, the dose can be repeated if desirable.

Administration of a recombinant AAV virion to the cell can be accomplished by any means, including simply contacting the particle, optionally contained in a desired liquid such as tissue culture medium, or a buffered saline solution, with the cells. The virion can be allowed to remain in contact with the cells for any desired length of time, and typically the virion is administered and allowed to remain indefinitely. For such in vitro methods, the virion can be administered to the cell by standard viral transduction methods, as known in the art and as exemplified herein. Titers of virus to administer can vary, particularly depending upon the cell type, but will be typical of that used for AAV transduction in general which is well known in the art. Additionally the titers used to transduce the particular cells in the present examples can be utilized.

The cells that can be transduced by the present recombinant AAV virions can include any desired cell, such as the following cells and cells derived from the following tissues, human as well as other mammalian tissues, such as primate, horse, sheep, goat, pig, dog, rat, and mouse and avian species: Adipocytes, Adenocyte, Adrenal cortex, Amnion, Aorta, Ascites, Astrocyte, Bladder, Bone, Bone marrow, Brain, Breast, Bronchus, Cardiac muscle, Cecum, Cervix, Chorion, Cochlear, Colon, Conjunctiva, Connective tissue, Cornea, Dermis, Duodenum, Embryonic stem cells, Endometrium, Endothelium, Endothelial cells, Epithelial tissue, Epithelial cells, Epidermis, Esophagus, Eye, Fascia, Fibroblasts, Foreskin, Gastric, Glial cells, Glioblast, Gonad, Hepatic cells, Histocyte, Hair cells in the inner ear, Ileum, Intestine, small Intestine, Jejunum, Keratinocytes, Kidney, Larynx, Leukocytes, Lipocyte, Liver, Lung, Lymph node, Lymphoblast, Lymphocytes, Macrophages, Mammary alveolar nodule, Mammary gland, Mastocyte, Maxilla, Melanocytes, Mesenchymal, Monocytes, Mouth, Myelin, Myoblasts Nervous tissue, Neuroblast, Neurons, Neuroglia, Osteoblasts, Osteogenic cells, Ovary, Palate, Pancreas, Papilloma, Peritoneum, Pituicytes, Pharynx, Placenta, Plasma cells, Pleura, Prostate, Rectum, Salivary gland, Skeletal muscle, Skin, Smooth muscle, Somatic, Spleen, Squamous, Stem cells, Stomach, Submandibular gland, Submaxillary gland, Synoviocytes, Testis, Thymus, Thyroid, Trabeculae, Trachea, Turbinate, Umbilical cord, Ureter, Uterus, and vestibular hair cells.

Stringency of hybridization is controlled by both temperature and salt concentration of either or both of the hybridization and washing steps. Typically, the stringency of hybridization to achieve selective hybridization involves hybridization in high ionic strength solution (6×SSC or 6× SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The washing temperatures can be used as described above to achieve selective stringency, as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987: 154:367, 1987). A preferable stringent hybridization condition for a DNA:DNA hybridization can be at about 68° C. (in aqueous solution) in 6×SSC or 6× SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

By the “suitability of an AAV vector for administration to a subject” is meant a determination of whether the AAV vector will elicit a neutralizing immune response upon administration to a particular subject. A vector that does not elicit a significant immune response is a potentially suitable vector, whereas a vector that elicits a significant, neutralizing immune response (e.g. at least 90%) is thus likely to be unsuitable for use in that subject. Significance of any detectable immune response is a standard parameter understood by the skilled artisan in the field. For example, one can incubate the subject's serum with the virus, then determine whether that virus retains its ability to transduce cells in culture. If such virus cannot transduce cells in culture, the vector likely has elicited a significant immune response.

Alternatively, or additionally, one skilled in the art could determine whether or not AAV administration would be suitable for a particular cell type of a subject. For example, the artisan could culture muscle cells in vitro and transduce the cells with AAV in the presence or absence of the subject's serum. If there is a reduction in transduction efficiency, this could indicate the presence of a neutralizing antibody or other factors that may inhibit transduction. Normally, greater than 90% inhibition would have to be observed in order to rule out the use of AAV-5 as a vector. However, this limitation could be overcome by treating the subject with an immunosuppressant that could block the factors inhibiting transduction.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

Previous research had demonstrated that Caco-2 and MDCK cells are model cell lines for the study of macromolecular transport via transcytosis. Furthermore these cell lines have been used to demonstrate transcytosis of both viruses and proteins. Therefore, to test if AAV can spread through tissue by transcytosis, 2×10⁸ DNA resistant particles of recombinant AAV2 (rAAV2) AAV4, AAV5, AAV6, BAAV suspended in 50 ul of medium were placed in the upper (apical) side of the transwell polycarbonate filter over a monolayer of cells each of the following cells Caco-2, MDCKI, MDCKII, Human primary airways epithelia cells (Airway), Human primary immortalized epithelial endometrial, Bovine brain primary endothelia cells (BBB), or HeLa. All cultures had TERs indicating the formation of tight junctions and polarized phenotype. After 3 hours of incubation the medium in the basal side of the transwell was collected and tested for the presence of transcytosed rAAV DNA. Viral DNA was extracted from 200 ul of basal medium and quantified by qPCR.

In these cell lines, transcytosis was observed with several AAV serotypes and appeared to be both serotype and tissue-specific (FIG. 1). Three hours after the addition of AAV to the apical surface of the cells, over 800,000 particles of AAV5 were present in the media on the basal lateral side of the trans-well insert of CaCo-2 cells, but not the MDCK, airway epithelia, endometrial, or BBB cells (FIG. 1). Similarly BAAV particles were detected in the media on the basal lateral side of the MDCK, airways epithelia, endometrial, and BBB cells but not the Caco-2 cells. Interestingly, AAV4 was detected in the basal lateral media of all cell types. No virus was detected in the basal lateral media when AAV2 was added to the apical surface in either cell type. AAV6 did not transcytose in any of cell types tested, and was not tested on airway epithelia or BBB. HeLa cells do not form barrier epithelia and were used as a control.

### Example 2

Previous work has demonstrated that transcytosis is a temperature dependent process than can be inhibited at 4° C. Transcytosis can also be inhibited by the addition of agents that selectively fix the plasma membrane. Recently the addition of tannic acid, a mild fixative agent, to the basal lateral surface blocked the transcytosis of GPI-anchored proteins to the apical surface (Polishchuk R, Nat Cell Biol. 2004. 6(4):297-307). Therefore the ability of this agent to block the transcytosis of AAV was tested. Treatment of the basal lateral surface of either Caco-2 or MDCK cells prior to virus addition to the apical surface blocked the accumulation of AAV5 or BAAV in the basal lateral media. Furthermore, quantification of the intracellular virus demonstrated inhibition of exocytosis by tannic acid treatment dramatically increase the amount of AAV DNA in the cell suggesting the viral particles detected in the basal lateral media are the result of an intracellular transport process and not a paracellular route.

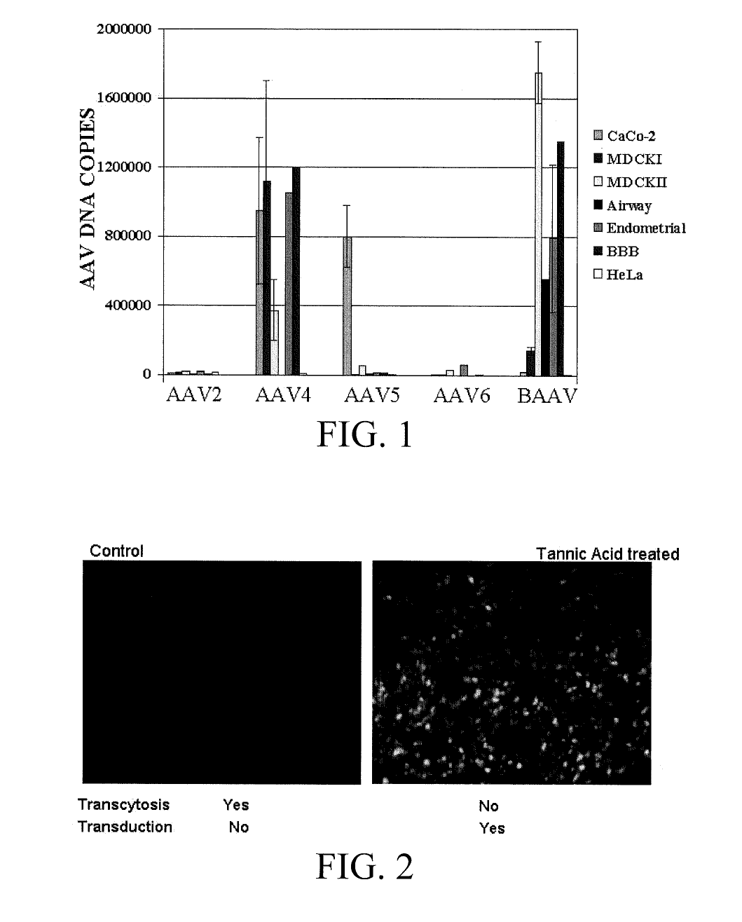

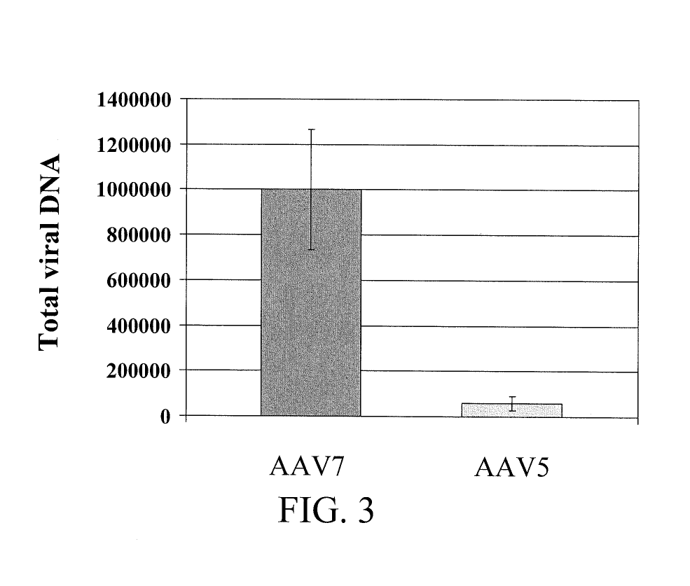

Treatment of the basal lateral surface of Human primary airways epithelial cell (HAE) with tannic acid blocked the transcytosis of BAAV or AAV4 vector containing a GFP expression cassette from the apical surface to the basal lateral (FIG. 2). Furthermore transduction dramatically increased when assayed at 24 hrs post inoculation. In contrast no change was observed in AAV2 transduction, which did not demonstrate any transcytosis activity and has limited binding activity on HAE.

### Example 3

To confirm the DNA detected in the basal lateral media was indeed extracted from intact virus, the material was tested for DNase resistance after treatment with heat, ionic detergent or protease. The addition of DNase alone or in combination with the ionic detergent deoxycholine had no effect on the viral DNA present in the media suggesting it was not free DNA or complexed in lipid vesicles. However, heating to 95° C. prior to treatment with DNAase completely degraded the viral DNA present in the media. This profile is identical to that of the input AAV particles and suggests the viral DNA is still encapsulated. Titration of the DNase resistant virus in the basal lateral media on Cos cells gave a similar particle to infectivity ratio to the input AAV particles.

While it would appear the AAV DNA detected in the basal lateral media is contained in intact particles, its presence on the basal lateral surface could be the result of lyses of the cells or disruption of the monolayer. Therefore the TER was carefully monitored throughout the course of these experiments and was not observed to decrease. To further confirm the integrity of the cell monolayer, mixing experiments were studied in which two viruses with different gene cassettes were added to the apical surface at the same time and three hours post addition the amount of each virus in the basal lateral media was quantified using QPCR specific for each cassette. Both BAAV and AAV5 were able to pass from the apical to the basal lateral surface of MDCK or Caco cells respectively but the AAV2 did not. Therefore the presence of viral particles in the basal lateral media does not appear to be the result of a disruption in the cell monolayer.

Taken together this data suggest that dependoviruses particles are capable of passing through barrier epithelia via transcytosis and the process is both serotype and cell type specific.

### Example 4

To further characterize the transcytosis activity observed with AAV5 and BAAV, transcytosis was quantified as both a time and concentration dependent event. After the addition of particles to the apical surface, samples were removed from the basal lateral media at different time points and the amount of virus was quantified by QPCR of the extracted DNA. Viral genomes could be detected as soon as 30 minutes after addition and steadily increased with time By 24 hrs, over ⅓ of the input recombinant AAV5, BAAVvirus added to Caco or MDCK cells respectively had been transported to the basal lateral surface. In contrast, none of the input AAV2 or adenovirus was detected on the basal lateral side after 24 hrs.

If transcytosis is an activity used by AAV to spread through tissue, this finding would help explain the lack of transduction of barrier epithelia reported with some isolates of AAV. Primary human bronchial airway epithelia (HAE) are known to transport albumin from the apical to the basal lateral surface by receptor-mediated transcytosis in vivo. While the interaction of BAAV with primary HAE has not been investigated, AAV4, 5 are reported to bind to HAE, however, for AAV4, this interaction does not result in transduction. Because of the interaction of AAV4 with O-link sialic acid, it was proposed, and has been demonstrated, that mucins, which contained large amounts of O-linked sialic acid and are expressed on the apical surface of RAE, can block AAV4 transduction. Alternatively the lack of transduction could be the result of transcytosis of the virus through the tissue.

To test this hypothesis, AAV2, 4, 5, BAAV were added to the apical surface of confluent monolayer cultures of primary human bronchial airway and transcytosis to the basal lateral surface was measured by QPCR after 3 hrs. All cultures had high TERs and expressed ciliated structures on their apical surface. Highly differentiated HAE cultures in contrast to immature cultures are resistant to transduction by adenoviral vectors due to a lack of integrin expression that is necessary for adenovirus entry.

Of the 4 AAVs tested for transcytosis, AAV4 and BAAV were detected in the basal lateral media. No transport of AAV2 or AAV5 was detected. As a control, adenovirus also was tested for transcytosis activity in the HAE cultures, but no transport was detected.

### Example 5

Epithelial cells that line the genitourinary tract form an important epithelial barrier layer and can transport proteins by transcytosis. AAV2, 4, 5 or BAAV were therefore tested to determine for the ability to penetrate this barrier epithelial layer by transcytosis. A well-characterized model of endometrial cells has been reported by Kyo et al. Following addition of the 4 AAVs to the apical surface, BAAV and AAV4 could be detected in the basal lateral media when assayed at 3hrs post inoculation (FIG. 1).

### Example 6

Most AAVs were identified originally as contaminants of laboratory stocks of adenovirus, thus our understanding of their natural biology, cell tropism, and knowledge the cellular components required for virus entry is limited. For AAV5, in addition to N-linked sialic acid, the platelet derived growth factor (PDGF) receptors were identified as protein receptors for AAV5 (Di Pasquale et al., Nat Med. 2003 October; 9(10):1306-12). This interaction was confirmed by modulation of PDGFR expression by transfection of expression plasmids, inhibitor treatment, or competition experiments with the extracellular domain of PDGFRa. Likewise AAV5 transduction could be blocked with sialolactosamine conjugates kaludov et al 2001.

Previous research had demonstrated that transcytosis is actin dependent and occurs by a caviolin mediated pathway. Furthermore transcytosis can be blocked by treatment with tannic acid. Therefore to better characterize the transcytosis pathway utilized by AAV5 in Caco cells the cells were treated with a panel of agents known to block either transcytosis in other systems or AAV5 mediated transduction. It was noted that AAV5 transcytosis could be inhibited by filipin and nocozodol as well as treatment with tannic acid.

Caco cells, which actively transcytosis AAV5, are not reported to express PDGFR and are not transduced by AAV5. In agreement, competition experiments with sPDGFRa had little effect on AAV5 transcytosis. Furthermore, competition experiments with 200 ug/ml sialolactosamine or 200 ug/ml heparin did not inhibited AAV5 transcytosis.

Both BSA and transferrin are reported to transcytosis through Caco cells via distinct receptor mediated pathways. However competition with either agent did not inhibit AAV5 transcytosis suggesting the AAV5 could use a distinct pathway.

In addition to confirming the intracellular nature of AAV5 transcytosis in Caco cells, the above experiments suggest that AAV5 transcytosis is occurring by a pathway independent of the one described for transduction. To confirm this Caco cells were stably transfected with PDGFRa and assayed for both transcytosis and transduction activity. Caco cells were not permissive for AAV5 transduction, however transduction dramatically increase following stable expression of PDGFRa. In contrast only a minor increase in transcytosis activity was detected in the Caco/PDGFRa cells.

Throughout this application, various publications are referenced. The disclosures of these publications in their entireties are hereby incorporated by reference into this application in order to more fully describe the state of the art to which this invention pertains.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1-68. (canceled)

69. A method to transduce an epithelial cell, the method comprising administering to an epithelial cell an inhibitor of exocytosis and an AAV vector comprising a heterologous nucleic acid, wherein the AAV vector transcytoses the epithelial cell in the absence of an exocytosis inhibitor, and wherein the epithelial cell is resistant to transduction by the AAV vector in the absence of an exocytosis inhibitor.

70. The method of claim 69, wherein the AAV vector is selected from the group consisting of an AAV4 vector, an AAV5 vector, and a BAAV vector.

71. The method of claim 69, wherein the epithelial cell is selected from the group consisting of a gut epithelial cell, a kidney epithelial cell, a lung epithelial cell, and a vascular endothelial cell.

72. The method of claim 69, wherein the epithelial cell is selected from the group consisting of a gut epithelial cell and a lung epithelial cell.

73. The method of claim 69, wherein the AAV vector is an AAV5 vector and wherein the epithelial cell is a gut epithelial cell.

74. The method of claim 69, wherein the AAV vector is an AAV5 vector and wherein the epithelial cell is a human gut epithelial cell.

75. The method of claim 73, wherein the AAV5 vector is administered orally to a patient.

76. The method of claim 69, wherein the AAV vector is a BAAV vector, and wherein the epithelial cell is an airway epithelial cell.

77. The method of claim 69, wherein the AAV vector is a BAAV vector and wherein the epithelial cell is a human airway epithelial cell.

78. The method of claim 76, wherein the BAAV vector is administered via aerosol delivery to a patient.

79. The method of claim 69, wherein the AAV vector is a BAAV vector and wherein the epithelial cell is a kidney epithelial cell.

80. The method of claim 69, wherein the AAV vector is a BAAV vector and wherein the epithelial cell is a human kidney epithelial cell.

81. The method of claim 69, wherein the AAV vector is a BAAV vector and wherein the epithelial cell is a vascular endothelial cell.

82. The method of claim 69, wherein the AAV vector is a BAAV vector and wherein the epithelial cell is a human vascular endothelial cell.

83. The method of claim 69, wherein the AAV vector is an AAV4 vector, and wherein the epithelial cell is an airway epithelial cell.

84. The method of claim 69, wherein the AAV vector is an AAV4 vector and wherein the epithelial cell is a human airway epithelial cell.

85. The method of claim 83, wherein the AAV4 vector is administered via aerosol delivery to a patient.

86. The method of claim 69, wherein the AAV vector is a AAV4 vector and wherein the epithelial cell is a kidney epithelial cell.

87. The method of claim 69, wherein the AAV vector is a AAV4 vector and wherein the epithelial cell is a human kidney epithelial cell.

88. The method of claim 69, wherein the AAV vector is an AAV4 vector and wherein the epithelial cell is a vascular endothelial cell.

89. The method of claim 69, wherein the AAV vector is an AAV4 vector and wherein the epithelial cell is a human vascular endothelial cell.

90. The method of claim 69, wherein the inhibitor of exocytosis is a chemical modifier.

91. The method of claim 69, wherein the inhibitor of exocytosis is selected from the group consisting of tannic acid, filipin and nocodazole.

92. The method of claim 69, wherein the inhibitor of exocytosis is tannic acid.

93. A method of delivering a heterologous nucleic acid to human gut epithelial cells, comprising delivering to the cells an inhibitor of exocytosis and an AAV5 vector comprising the heterologous nucleic acid.

94. A method of delivering a heterologous nucleic acid to human epithelial cells, comprising delivering to the cells an inhibitor of exocytosis and a BAAV vector comprising the heterologous nucleic acid, wherein the cells are selected from the group consisting of human airway epithelial cells, human kidney epithelial cells, and human vascular endothelial cells.

95. A method of delivering a heterologous nucleic acid to human epithelial cells, comprising delivering to the cells an inhibitor of exocytosis and an AAV4 vector comprising the heterologous nucleic acid, wherein the cells are selected from the group consisting of human airway epithelial cells, human kidney epithelial cells, and human vascular endothelial cells.


SEQUENCE LISTING (72 sequences)

SEQ ID NO: 1 - DNA, 4768 bp, Artificial Sequence
  Description of Artificial Sequence:  note = synthetic construct
  misc_feature (3009)..(3009): n = A,T,C or G
ttggccactccctctatgcgcgctcgctcactcactcggccctggagaccaaaggtctccagactgccggcctctggccggcagggccgagtgagtgagcgagcgcgcatagagggagtggccaactccatcatctaggtttgcccactgacgtcaatgtgacgtcctagggttagggaggtccctgtattagcagtcacgtgagtgtcgtatttcgcggagcgtagcggagcgcataccaagctgccacgtcacagccacgtggtccgtttgcgacagtttgcgacaccatgtggtcaggagggtatataaccgcgagtgagccagcgaggagctccattttgcccgcgaattttgaacgagcagcagccatgccggggttctacgagatcgtgctgaaggtgcccagcgacctggacgagcacctgcccggcatttctgactcttttgtgagctgggtggccgagaaggaatgggagctgccgccggattctgacatggacttgaatctgattgagcaggcacccctgaccgtggccgaaaagctgcaacgcgagttcctggtcgagtggcgccgcgtgagtaaggccccggaggccctcttctttgtccagttcgagaagggggacagctacttccacctgcacatcctggtggagaccgtgggcgtcaaatccatggtggtgggccgctacgtgagccagattaaagagaagctggtgacccgcatctaccgcggggtcgagccgcagcttccgaactggttcgcggtgaccaagacgcgtaatggcgccggaggcgggaacaaggtggtggacgactgctacatccccaactacctgctccccaagacccagcccgagctccagtgggcgtggactaacatggaccagtatataagcgcctgtttgaatctcgcggagcgtaaacggctggtggcgcagcatctgacgcacgtgtcgcagacgcaggagcagaacaaggaaaaccagaaccccaattctgacgcgccggtcatcaggtcaaaaacctccgccaggtacatggagctggtcgggtggctggtggaccgcgggatcacgtcagaaaagcaatggatccaggaggaccaggcgtcctacatctccttcaacgccgcctccaactcgcggtcacaaatcaaggccgcgctggacaatgcctccaaaatcatgagcctgacaaagacggctccggactacctggtgggccagaacccgccggaggacatttccagcaaccgcatctaccgaatcctcgagatgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtgaactggaccaatgagaactttccgttcaacgattgcgtcgacaagatggtgatctggtgggaggagggcaagatgacggccaaggtcgtagagagcgccaaggccatcctgggcggaagcaaggtgcgcgtggaccaaaagtgcaagtcatcggcccagatcgacccaactcccgtgatcgtcacctccaacaccaacatgtgcgcggtcatcgacggaaactcgaccaccttcgagcaccaacaaccactccaggaccggatgttcaagttcgagctcaccaagcgcctggagcacgactttggcaaggtcaccaagcaggaagtcaaagactttttccggtgggcgtcagatcacgtgaccgaggtgactcacgagttttacgtcagaaagggtggagctagaaagaggcccgcccccaatgacgcagatataagtgagcccaagcgggcctgtccgtcagttgcgcagccatcgacgtcagacgcggaagctccggtggactacgcggacaggtaccaaaacaaatgttctcgtcacgtgggtatgaatctgatgctttttccctgccggcaatgcgagagaatgaatcagaatgtggacatttgcttcacgcacggggtcatggactgtgccgagtgcttccccgtgtcagaatctcaacccgtgtctgtcgtcagaaagcggacgtatcagaaactgtgtccgattcatcacatcatggggagggcgcccgaggtggcctgctcggcctgcgaactggccaatgtggacttggatgactgtgacatggaacaataaatgactcaaaccagatatgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacaccgtttgggggcaacctcggcagagcagtcttccaggccaaaaagagggttcttgaacctcttggtctggttgagcaagcgggtgagacggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggsggacaaggtgccgatggagtgggtaatgcctcgggtgattggcattgcgattccacctggtctgagggccacgtcacgaccaccagcaccagaacctgggtcttgcccacctacaacaaccacctntacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctgtaataacctgttaatcaataaaccggtttattcgtttcagttgaactttggtctccgtgtccttcttatcttatctcgtttccatggctactgcgtacataagcagcggcctgcggcgcttgcgcttcgcggtttacaactgccggttaatcagtaacttctggcaaaccatgatgatggagttggccactccctctatgcgcgctcgctcactcactcggccctggagaccaaaggtctccagactgccggcctctggccggcagggccgagtgagtgagcgagcgcgcatagagggagtggccaa

SEQ ID NO: 2 - protein, 623 aa, Artificial Sequence
  Description of Artificial Sequence:  note = synthetic construct
MPGFYEIVLKVPSDLDEHLPGISDSFVSWVAEKEWELPPDSDMDLNLIEQAPLTVAEKLQREFLVEWRRVSKAPEALFFVQFEKGDSYFHLHILVETVGVKSMVVGRYVSQIKEKLVTRIYRGVEPQLPNWFAVTKTRNGAGGGNKVVDDCYIPNYLLPKTQPELQWAWTNMDQYISACLNLAERKRLVAQHLTHVSQTQEQNKENQNPNSDAPVIRSKTSARYMELVGWLVDRGITSEKQWIQEDQASYISFNAASNSRSQIKAALDNASKIMSLTKTAPDYLVGQNPPEDISSNRIYRILEMNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTKRLEHDFGKVTKQEVKDFFRWASDHVTEVTHEFYVRKGGARKRPAPNDADISEPKRACPSVAQPSTSDAEAPVDYADRYQNKCSRHVGMNLMLFPCRQCERMNQNVDICFTHGVMDCAECFPVSESQPVSVVRKRTYQKLCPIHHIMGRAPEVACSACELANVDLDDCDMEQ

SEQ ID NO: 3 - protein, 2495 aa, Artificial Sequence
  Description of Artificial Sequence:  note = synthetic construct
ATGCCGGGGTTCTACGAGATCGTGCTGAAGGTGCCCAGCGACCTGGACMPGFYEIVLKVPSDLDGAGCACCTGCCCGGCATTTCTGACTCTTTTGTGAGCTGGGTGGCCGAGEHLPGISDSFVSWVAEAAGGAATGGGAGCTGCCGCCGGATTCTGACATGGACTTGAATCTGATTKEWELPPDSDMDLNLIGAGCAGGCACCCCTGACCGTGGCCGAAAAGCTGCAACGCGAGTTCCTGEQAPLTVAEKLQREFLGTCGAGTGGCGCCGCGTGAGTAAGGCCCCGGAGGCCCTCTTCTTTGTCVEWRRVSKAPEALFFVCAGTTCGAGAAGGGGGACAGCTACTTCCACCTGCACATCCTGGTGGAGQFEKGDSYFHLHILVEACCGTGGGCGTCAAATCCATGGTGGTGGGCCGCTACGTGAGCCAGATTTVGVKSMVVGRYVSQIAAAGAGAAGCTGGTGACCCGCATCTACCGCGGGGTCGAGCCGCAGCTTKEKLVTRIYRGVEPQLCCGAACTGGTTCGCGGTGACCAAGACGCGTAATGGCGCCGGAGGCGGGPNWFAVTKTRNGAGGGAACAAGGTGGTGGACGACTGCTACATCCCCAACTACCTGCTCCCCAAGNKVVDDCYIPNYLLPKACCCAGCCCGAGCTCCAGTGGGCGTGGACTAACATGGACCAGTATATATQPELQWAWTNMDQYIAGCGCCTGTTTGAATCTCGCGGAGCGTAAACGGCTGGTGGCGCAGCATSACLNLAERKRLVAQHCTGACGCACGTGTCGCAGACGCAGGAGCAGAACAAGGAAAACCAGAACLTHVSQTQEQNKENQNCCCAATTCTGACGCGCCGGTCATCAGGTCAAAAACCTCCGCCAGGTACPNSDAPVIRSKTSARYATGGAGCTGGTCGGGTGGCTGGTGGACCGCGGGATCACGTCAGAAAAGMELVGWLVDRGITSEKCAATGGATCCAGGAGGACCAGGCGTCCTACATCTCCTTCAACGCCGCCQWIQEDQASYISFNAATCCAACTCGCGGTCACAAATCAAGGCCGCGCTGGACAATGCCTCCAAASNSRSQIKAALDNASKATCATGAGCCTGACAAAGACGGCTCCGGACTACCTGGTGGGCCAGAACIMSLTKTAPDYLVGQNCCGCCGGAGGACATTTCCAGCAACCGCATCTACCGAATCCTCGAGATGPPEDISSNRIYRILEMAACGGGTACGATCCGCAGTACGCGGCCTCCGTCTTCCTGGGCTGGGCGNGYDPQYAASVFLGWACAAAAGAAGTTCGGGAAGAGGAACACCATCTGGCTCTTTGGGCCGGCCQKKFGKRNTIWLFGPAACGACGGGTAAAACCAACATCGCGGAAGCCATCGCCCACGCCGTGCCCTTGKTNIAEAIAHAVPTTCTACGGCTGCGTGAACTGGACCAATGAGAACTTTCCGTTCAACGATFYGCVNWTNENFPFNDTGCGTCGACAAGATGGTGATCTGGTGGGAGGAGGGCAAGATGACGGCCCVDKMVIWWEEGKMTAAAGGTCGTAGAGAGCGCCAAGGCCATCCTGGGCGGAAGCAAGGTGCGCKVVESAKAILGGSKVRGTGGACCAAAAGTGCAAGTCATCGGCCCAGATCGACCCAACTCCCGTGVDQKCKSSAQIDPTPVATCGTCACCTCCAACACCAACATGTGCGCGGTCATCGACGGAAACTCGIVTSNTNMCAVIDGNSACCACCTTCGAGCACCAACAACCACTCCAGGACCGGATGTTCAAGTTCTTFEHQQPLQDRMFKFGAGCTCACCAAGCGCCTGGAGCACGACTTTGGCAAGGTCACCAAGCAGELTKRLEHDFGKVTKQGAAGTCAAAGACTTTTTCCGGTGGGCGTCAGATCACGTGACCGAGGTGEVKDFFRWASDHVTEVACTCACGAGTTTTACGTCAGAAAGGGTGGAGCTAGAAAGAGGCCCGCCTHEFYVRKGGARKRPACCCAATGACGCAGATATAAGTGAGCCCAAGCGGGCCTGTCCGTCAGTTPNDADISEPKRACPSVGCGCAGCCATCGACGTCAGACGCGGAAGCTCCGGTGGACTACGCGGACAQPSTSDAEAPVDYADAGGTACCAAAACAAATGTTCTCGTCACGTGGGTATGAATCTGATGCTTRYQNKCSRHVGMNLMLTTTCCCTGCCGGCAATGCGAGAGAATGAATCAGAATGTGGACATTTGCFPCRQCERMNQNVDICTTCACGCACGGGGTCATGGACTGTGCCGAGTGCTTCCCCGTGTCAGAAFTHGVMDCAECFPVSETCTCAACCCGTGTCTGTCGTCAGAAAGCGGACGTATCAGAAACTGTGTSQPVSVVRKRTYQKLCCCGATTCATCACATCATGGGGAGGGCGCCCGAGGTGGCCTGCTCGGCCPIHHIMGRAPEVACSATGCGAACTGGCCAATGTGGACTTGGATGACTGTGACATGGAACAATAACELANVDLDDCDMEQ

SEQ ID NO: 4 - protein, 734 aa, Artificial Sequence
  Description of Artificial Sequence:  note = synthetic construct
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 5 - DNA, 2208 bp, Artificial Sequence
  Description of Artificial Sequence:  note = synthetic construct
  misc_feature (750)..(750): n = A,T,C or G
atgactgacggttaccttccagattggctagaggacaacctctctgaaggcgttcgagagtggtgggcgctgcaacctggagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggsggacaaggtgccgatggagtgggtaatgcctcgggtgattggcattgcgattccacctggtctgagggccacgtcacgaccaccagcaccagaacctgggtcttgcccacctacaacaaccacctntacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctgtaataa

SEQ ID NO: 6 - DNA, 125 bp, Artificial Sequence
  Description of Artificial Sequence:  note = synthetic construct
ttggccactccctctatgcgcgctcgctcactcactcggccctggagaccaaaggtctccagactgccggcctctggccggcagggccgagtgagtgagcgagcgcgcatagagggagtggccaa

SEQ ID NO: 7 - DNA, 245 bp, Artificial Sequence
  Description of Artificial Sequence:  note = synthetic construct
ctccatcatctaggtttgcccactgacgtcaatgtgacgtcctagggttagggaggtccctgtattagcagtcacgtgagtgtcgtatttcgcggagcgtagcggagcgcataccaagctgccacgtcacagccacgtggtccgtttgcgacagtttgcgacaccatgtggtcaggagggtatataaccgcgagtgagccagcgaggagctccattttgcccgcgaattttgaacgagcagcagc

SEQ ID NO: 8 - protein, 313 aa, Artificial Sequence
  Description of Artificial Sequence:  note = synthetic construct
  VARIANT (313)..(313): Xaa = Any Amino Acid
MELVGWLVDRGITSEKQWIQEDQASYISFNAASNSRSQIKAALDNASKIMSLTKTAPDYLVGQNPPEDISSNRIYRILEMNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTKRLEHDFGKVTKQEVKDFFRWASDHVTEVTHEFYVRKGGARKRPAPNDADISEPKRACPSVAQPSTSDAEAPVDYADRLARGQPLX

SEQ ID NO: 9 - protein, 399 aa, Artificial Sequence
  Description of Artificial Sequence:  note = synthetic construct
MELVGWLVDRGITSEKQWIQEDQASYISFNAASNSRSQIKAALDNASKIMSLTKTAPDYLVGQNPPEDISSNRIYRILEMNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTKRLEHDFGKVTKQEVKDFFRWASDHVTEVTHEFYVRKGGARKRPAPNDADISEPKRACPSVAQPSTSDAEAPVDYADRYQNKCSRHVGMNLMLFPCRQCERMNQNVDICFTHGVMDCAECFPVSESQPVSVVRKRTYQKLCPIHHIMGRAPEVACSACELANVDLDDCDMEQ

SEQ ID NO: 10 - protein, 537 aa, Artificial Sequence
  Description of Artificial Sequence:  note = synthetic construct
  VARIANT (537)..(537): Xaa = Any Amino Acid
MPGFYEIVLKVPSDLDEHLPGISDSFVSWVAEKEWELPPDSDMDLNLIEQAPLTVAEKLQREFLVEWRRVSKAPEALFFVQFEKGDSYFHLHILVETVGVKSMVVGRYVSQIKEKLVTRIYRGVEPQLPNWFAVTKTRNGAGGGNKVVDDCYIPNYLLPKTQPELQWAWTNMDQYISACLNLAERKRLVAQHLTHVSQTQEQNKENQNPNSDAPVIRSKTSARYMELVGWLVDRGITSEKQWIQEDQASYISFNAASNSRSQIKAALDNASKIMSLTKTAPDYLVGQNPPEDISSNRIYRILEMNGYDPQYAASVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTKRLEHDFGKVTKQEVKDFFRWASDHVTEVTHEFYVRKGGARKRPAPNDADISEPKRACPSVAQPSTSDAEAPVDYADRLARGQPLX

SEQ ID NO: 11 - protein, 623 aa, Artificial Sequence; identical to SEQ ID NO: 2
  Description of Artificial Sequence:  note = synthetic construct

SEQ ID NO: 12 - DNA, 939 bp, Artificial Sequence
  Description of Artificial Sequence:  note = synthetic construct
atggagctggtcgggtggctggtggaccgcgggatcacgtcagaaaagcaatggatccaggaggaccaggcgtcctacatctccttcaacgccgcctccaactcgcggtcacaaatcaaggccgcgctggacaatgcctccaaaatcatgagcctgacaaagacggctccggactacctggtgggccagaacccgccggaggacatttccagcaaccgcatctaccgaatcctcgagatgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtgaactggaccaatgagaactttccgttcaacgattgcgtcgacaagatggtgatctggtgggaggagggcaagatgacggccaaggtcgtagagagcgccaaggccatcctgggcggaagcaaggtgcgcgtggaccaaaagtgcaagtcatcggcccagatcgacccaactcccgtgatcgtcacctccaacaccaacatgtgcgcggtcatcgacggaaactcgaccaccttcgagcaccaacaaccactccaggaccggatgttcaagttcgagctcaccaagcgcctggagcacgactttggcaaggtcaccaagcaggaagtcaaagactttttccggtgggcgtcagatcacgtgaccgaggtgactcacgagttttacgtcagaaagggtggagctagaaagaggcccgcccccaatgacgcagatataagtgagcccaagcgggcctgtccgtcagttgcgcagccatcgacgtcagacgcggaagctccggtggactacgcggacagattggctagaggacaacctctctga

SEQ ID NO: 13 - DNA, 1197 bp, Artificial Sequence
  Description of Artificial Sequence:  note = synthetic construct
atggagctggtcgggtggctggtggaccgcgggatcacgtcagaaaagcaatggatccaggaggaccaggcgtcctacatctccttcaacgccgcctccaactcgcggtcacaaatcaaggccgcgctggacaatgcctccaaaatcatgagcctgacaaagacggctccggactacctggtgggccagaacccgccggaggacatttccagcaaccgcatctaccgaatcctcgagatgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtgaactggaccaatgagaactttccgttcaacgattgcgtcgacaagatggtgatctggtgggaggagggcaagatgacggccaaggtcgtagagagcgccaaggccatcctgggcggaagcaaggtgcgcgtggaccaaaagtgcaagtcatcggcccagatcgacccaactcccgtgatcgtcacctccaacaccaacatgtgcgcggtcatcgacggaaactcgaccaccttcgagcaccaacaaccactccaggaccggatgttcaagttcgagctcaccaagcgcctggagcacgactttggcaaggtcaccaagcaggaagtcaaagactttttccggtgggcgtcagatcacgtgaccgaggtgactcacgagttttacgtcagaaagggtggagctagaaagaggcccgcccccaatgacgcagatataagtgagcccaagcgggcctgtccgtcagttgcgcagccatcgacgtcagacgcggaagctccggtggactacgcggacaggtaccaaaacaaatgttctcgtcacgtgggtatgaatctgatgctttttccctgccggcaatgcgagagaatgaatcagaatgtggacatttgcttcacgcacggggtcatggactgtgccgagtgcttccccgtgtcagaatctcaacccgtgtctgtcgtcagaaagcggacgtatcagaaactgtgtccgattcatcacatcatggggagggcgcccgaggtggcctgctcggcctgcgaactggccaatgtggacttggatgactgtgacatggaacaa

SEQ ID NO: 14 - DNA, 1611 bp, Artificial Sequence
  Description of Artificial Sequence:  note = synthetic construct
atgccggggttctacgagatcgtgctgaaggtgcccagcgacctggacgagcacctgcccggcatttctgactcttttgtgagctgggtggccgagaaggaatgggagctgccgccggattctgacatggacttgaatctgattgagcaggcacccctgaccgtggccgaaaagctgcaacgcgagttcctggtcgagtggcgccgcgtgagtaaggccccggaggccctcttctttgtccagttcgagaagggggacagctacttccacctgcacatcctggtggagaccgtgggcgtcaaatccatggtggtgggccgctacgtgagccagattaaagagaagctggtgacccgcatctaccgcggggtcgagccgcagcttccgaactggttcgcggtgaccaagacgcgtaatggcgccggaggcgggaacaaggtggtggacgactgctacatccccaactacctgctccccaagacccagcccgagctccagtgggcgtggactaacatggaccagtatataagcgcctgtttgaatctcgcggagcgtaaacggctggtggcgcagcatctgacgcacgtgtcgcagacgcaggagcagaacaaggaaaaccagaaccccaattctgacgcgccggtcatcaggtcaaaaacctccgccaggtacatggagctggtcgggtggctggtggaccgcgggatcacgtcagaaaagcaatggatccaggaggaccaggcgtcctacatctccttcaacgccgcctccaactcgcggtcacaaatcaaggccgcgctggacaatgcctccaaaatcatgagcctgacaaagacggctccggactacctggtgggccagaacccgccggaggacatttccagcaaccgcatctaccgaatcctcgagatgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtgaactggaccaatgagaactttccgttcaacgattgcgtcgacaagatggtgatctggtgggaggagggcaagatgacggccaaggtcgtagagagcgccaaggccatcctgggcggaagcaaggtgcgcgtggaccaaaagtgcaagtcatcggcccagatcgacccaactcccgtgatcgtcacctccaacaccaacatgtgcgcggtcatcgacggaaactcgaccaccttcgagcaccaacaaccactccaggaccggatgttcaagttcgagctcaccaagcgcctggagcacgactttggcaaggtcaccaagcaggaagtcaaagactttttccggtgggcgtcagatcacgtgaccgaggtgactcacgagttttacgtcagaaagggtggagctagaaagaggcccgcccccaatgacgcagatataagtgagcccaagcgggcctgtccgtcagttgcgcagccatcgacgtcagacgcggaagctccggtggactacgcggacagattggctagaggacaacctctctga

SEQ ID NO: 15 - DNA, 1872 bp, Artificial Sequence
  Description of Artificial Sequence:  note = synthetic construct
atgccggggttctacgagatcgtgctgaaggtgcccagcgacctggacgagcacctgcccggcatttctgactcttttgtgagctgggtggccgagaaggaatgggagctgccgccggattctgacatggacttgaatctgattgagcaggcacccctgaccgtggccgaaaagctgcaacgcgagttcctggtcgagtggcgccgcgtgagtaaggccccggaggccctcttctttgtccagttcgagaagggggacagctacttccacctgcacatcctggtggagaccgtgggcgtcaaatccatggtggtgggccgctacgtgagccagattaaagagaagctggtgacccgcatctaccgcggggtcgagccgcagcttccgaactggttcgcggtgaccaagacgcgtaatggcgccggaggcgggaacaaggtggtggacgactgctacatccccaactacctgctccccaagacccagcccgagctccagtgggcgtggactaacatggaccagtatataagcgcctgtttgaatctcgcggagcgtaaacggctggtggcgcagcatctgacgcacgtgtcgcagacgcaggagcagaacaaggaaaaccagaaccccaattctgacgcgccggtcatcaggtcaaaaacctccgccaggtacatggagctggtcgggtggctggtggaccgcgggatcacgtcagaaaagcaatggatccaggaggaccaggcgtcctacatctccttcaacgccgcctccaactcgcggtcacaaatcaaggccgcgctggacaatgcctccaaaatcatgagcctgacaaagacggctccggactacctggtgggccagaacccgccggaggacatttccagcaaccgcatctaccgaatcctcgagatgaacgggtacgatccgcagtacgcggcctccgtcttcctgggctgggcgcaaaagaagttcgggaagaggaacaccatctggctctttgggccggccacgacgggtaaaaccaacatcgcggaagccatcgcccacgccgtgcccttctacggctgcgtgaactggaccaatgagaactttccgttcaacgattgcgtcgacaagatggtgatctggtgggaggagggcaagatgacggccaaggtcgtagagagcgccaaggccatcctgggcggaagcaaggtgcgcgtggaccaaaagtgcaagtcatcggcccagatcgacccaactcccgtgatcgtcacctccaacaccaacatgtgcgcggtcatcgacggaaactcgaccaccttcgagcaccaacaaccactccaggaccggatgttcaagttcgagctcaccaagcgcctggagcacgactttggcaaggtcaccaagcaggaagtcaaagactttttccggtgggcgtcagatcacgtgaccgaggtgactcacgagttttacgtcagaaagggtggagctagaaagaggcccgcccccaatgacgcagatataagtgagcccaagcgggcctgtccgtcagttgcgcagccatcgacgtcagacgcggaagctccggtggactacgcggacaggtaccaaaacaaatgttctcgtcacgtgggtatgaatctgatgctttttccctgccggcaatgcgagagaatgaatcagaatgtggacatttgcttcacgcacggggtcatggactgtgccgagtgcttccccgtgtcagaatctcaacccgtgtctgtcgtcagaaagcggacgtatcagaaactgtgtccgattcatcacatcatggggagggcgcccgaggtggcctgctcggcctgcgaactggccaatgtggacttggatgactgtgacatggaacaataa

SEQ ID NO: 16 - protein, 598 aa, Artificial Sequence
  Description of Artificial Sequence:  note = synthetic construct
TAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 17 - DNA, 1800 bp, Artificial Sequence
  Description of Artificial Sequence:  note = synthetic construct
  misc_feature (342)..(342): n = A,T,C or G
acggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggsggacaaggtgccgatggagtgggtaatgcctcgggtgattggcattgcgattccacctggtctgagggccacgtcacgaccaccagcaccagaacctgggtcttgcccacctacaacaaccacctntacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctgtaataa

SEQ ID NO: 18 - protein, 544 aa, Artificial Sequence
  Description of Artificial Sequence:  note = synthetic construct
MSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 19 - DNA, 1617 bp, Artificial Sequence
  Description of Artificial Sequence:  note = synthetic construct
  misc_feature (162)..(162): n = A,T,C or G
atgcgtgcagcagctggcggagctgcagtcgagggsggacaaggtgccgatggagtgggtaatgcctcgggtgattggcattgcgattccacctggtctgagggccacgtcacgaccaccagcaccagaacctgggtcttgcccacctacaacaaccacctntacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctgtaa

SEQ ID NO: 20 - DNA, 129 bp, Artificial Sequence
  Description of Artificial Sequence:  note = synthetic construct
ttggccactccctctatgcgcgctcgctcactcactcggccctgcggccagaggccggcagtctggagacctttggtgtccagggcagggccgagtgagtgagcgagcgcgcatagagggagtggccaa

SEQ ID NO: 21 - DNA, 35 bp, Artificial Sequence
  Description of Artificial Sequence:  note = synthetic construct
tctagtctagacttggccactccctctctgcgcgc

SEQ ID NO: 22 - DNA, 34 bp, Artificial Sequence
  Description of Artificial Sequence:  note = synthetic construct
aggccttaagagcagtcgtccaccaccttgttcc

SEQ ID NO: 23 - DNA, 4652 bp, Artificial Sequence
  Description of Artificial Sequence:  note = synthetic construct
tggcactctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgccacactctcaagcaagggggttttgtaagcagtgatgtcataatgatgtaatgcttattgtcacgcgatagttaatgattaacagtcatgtgatgtgttttatccaataggaagaaagcgcgcgtatgagttctcgcgagacttccggggtataaaagaccgagtgaacgagcccgccgccattctttgctctggactgctagaggaccctcgctgccatggctaccttctatgaagtcattgttcgcgtcccatttgacgtggaggaacatctgcctggaatttctgacagctttgtggactgggtaactggtcaaatttgggagctgcctccagagtcagatttaaatttgactctggttgaacagcctcagttgacggtggctgatagaattcgccgcgtgttcctgtacgagtggaacaaattttccaagcaggagtccaaattctttgtgcagtttgaaaagggatctgaatattttcatctgcacacgcttgtggagacctccggcatctcttccatggtcctcggccgctacgtgagtcagattcgcgcccagctggtgaaagtggtcttccagggaattgaaccccagatcaacgactgggtcgccatcaccaaggtaaagaagggcggagccaataaggtggtggattctgggtatattcccgcctacctgctgccgaaggtccaaccggagcttcagtgggcgtggacaaacctggacgagtataaattggccgccctgaatctggaggagcgcaaacggctcgtcgcgcagtttctggcagaatcctcgcagcgctcgcaggaggcggcttcgcagcgtgagttctcggctgacccggtcatcaaaagcaagacttcccagaaatacatggcgctcgtcaactggctcgtggagcacggcatcacttccgagaagcagtggatccaggaaaatcaggagagctacctctccttcaactccaccggcaactctcggagccagatcaaggccgcgctcgacaacgcgaccaaaattatgagtctgacaaaaagcgcggtggactacctcgtggggagctccgttcccgaggacatttcaaaaaacagaatctggcaaatttttgagatgaatggctacgacccggcctacgcgggatccatcctctacggctggtgtcagcgctccttcaacaagaggaacaccgtctggctctacggacccgccacgaccggcaagaccaacatcgcggaggccatcgcccacactgtgcccttttacggctgcgtgaactggaccaatgaaaactttccctttaatgactgtgtggacaaaatgctcatttggtgggaggagggaaagatgaccaacaaggtggttgaatccgccaaggccatcctggggggctcaaaggtgcgggtcgatcagaaatgtaaatcctctgttcaaattgattctacccctgtcattgtaacttccaatacaaacatgtgtgtggtggtggatgggaattccacgacctttgaacaccagcagccgctggaggaccgcatgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctcaattggaattcaaggtatgattgcaaatgtgactatcatgctcaatttgacaacatttctaacaaatgtgatgaatgtgaatatttgaatcggggcaaaaatggatgtatctgtcacaatgtaactcactgtcaaatttgtcatgggattcccccctgggaaaaggaaaacttgtcagattttggggattttgacgatgccaataaagaacagtaaataaagcgagtagtcatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaacccattcatgtcgcataccctcaataaaccgtgtattcgtgtcagtaaaatactgcctcttgtggtcattcaatgaataacagcttacaacatctacaaaacctccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgcca

SEQ ID NO: 24 - protein, 390 aa, Artificial Sequence
  Description of Artificial Sequence:  note = synthetic construct
MALVNWLVEHGITSEKQWIQENQESYLSFNSTGNSRSQIKAALDNATKIMSLTKSAVDYLVGSSVPEDISKNRIWQIFEMNGYDPAYAGSILYGWCQRSFNKRNTVWLYGPATTGKTNIAEAIAHTVPFYGCVNWTNENFPFNDCVDKMLIWWEEGKMTNKVVESAKAILGGSKVRVDQKCKSSVQIDSTPVIVTSNTNMCVVVDGNSTTFEHQQPLEDRMFKFELTKRLPPDFGKITKQEVKDFFAWAKVNQVPVTHEFKVPRELAGTKGAEKSLKRPLGDVTNTSYKSLEKRARLSFVPETPRSSDVTVDPAPLRPLNWNSRYDCKCDYHAQFDNISNKCDECEYLNRGKNGCICHNVTHCQICHGIPPWEKENLSDFGDFDDANKEQ

SEQ ID NO: 25 - protein, 594 aa, Artificial Sequence
  Description of Artificial Sequence:  note = synthetic construct
EHLPGISDSFVDWVTGQIWELPPESDLNLTLVEQPQLTVADRIRRVFLYEWNKFSKQESKFFVQFEKGSEYFHLHTLVETSGISSMVLGRYVSQIRAQLVKVVFQGIEPQINDWVAITKVKKGGANKVVDSGYIPAYLLPKVQPELQWAWTNLDEYKLAALNLEERKRLVAQFLAESSQRSQEAASQREFSADPVIKSKTSQKYMALVNWLVEHGITSEKQWIQENQESYLSFNSTGNSRSQIKAALDNATKIMSLTKSAVDYLVGSSVPEDISKNRIWQIFEMNGYDPAYAGSILYGWCQRSFNKRNTVWLYGPATTGKTNIAEAIAHTVPFYGCVNWTNENFPFNDCVDKMLIWWEEGKMTNKVVESAKAILGGSKVRVDQKCKSSVQIDSTPVIVTSNTNMCVVVDGNSTTFEHQQPLEDRMFKFELTKRLPPDFGKITKQEVKDFFAWAKVNQVPVTHEFKVPRELAGTKGAEKSLKRPLGDVTNTSYKSLEKRARLSFVPETPRSSDVTVDPAPLRPLNWNSRYDCKCDYHAQFDNISNKCDECEYLNRGKNGCICHNVTHCQICHGIPPWEKENLSDFGDFDDANKEQ

SEQ ID NO: 26 - protein, 724 aa, Artificial Sequence
  Description of Artificial Sequence:  note = synthetic construct
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 27 - protein, 588 aa, Artificial Sequence
  Description of Artificial Sequence:  note = synthetic construct
TAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 28 - protein, 532 aa, Artificial Sequence
  Description of Artificial Sequence:  note = synthetic construct
MSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 29 - DNA, 2307 bp, Artificial Sequence
  Description of Artificial Sequence:  note = synthetic construct
aggctctcatttgttcccgagacgcctcgcagttcagacgtgactgttgatcccgctcctctgcgaccgctcaattggaattcaagtaaataaagcgagtagtcatgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaacccattcatgtcgcataccctcaataaa

SEQ ID NO: 30 - DNA, 2264 bp, Artificial Sequence
  Description of Artificial Sequence:  note = synthetic construct
aggctctcatttgttcccgagacgcctcgcagttcagacgtgactgttgatcccgctcctctgcgaccgctcaattggaattcaagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaacccattcatgtcgcataccctcaataaa

SEQ ID NO: 31 - DNA, 2264 bp, Artificial Sequence; identical to SEQ ID NO: 30
  Description of Artificial Sequence:  note = synthetic construct

SEQ ID NO: 32 - DNA, 1292 bp, Artificial Sequence
  Description of Artificial Sequence:  note = synthetic construct
agcgcaaacggctcgtcgcgcagtttctggcagaatcctcgcagcgctcgcaggaggcggcttcgcagcgtgagttctcggctgacccggtcatcaaaagcaagacttcccagaaatacatggcgctcgtcaactggctcgtggagcacggcatcacttccgagaagcagtggatccaggaaaatcaggagagctacctctccttcaactccaccggcaactctcggagccagatcaaggccgcgctcgacaacgcgaccaaaattatgagtctgacaaaaagcgcggtggactacctcgtggggagctccgttcccgaggacatttcaaaaaacagaatctggcaaatttttgagatgaatggctacgacccggcctacgcgggatccatcctctacggctggtgtcagcgctccttcaacaagaggaacaccgtctggctctacggacccgccacgaccggcaagaccaacatcgcggaggccatcgcccacactgtgcccttttacggctgcgtgaactggaccaatgaaaactttccctttaatgactgtgtggacaaaatgctcatttggtgggaggagggaaagatgaccaacaaggtggttgaatccgccaaggccatcctggggggctcaaaggtgcgggtcgatcagaaatgtaaatcctctgttcaaattgattctacccctgtcattgtaacttccaatacaaacatgtgtgtggtggtggatgggaattccacgacctttgaacaccagcagccgctggaggaccgcatgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctcaattggaattcaaggtatgattgcaaatgtgactatcatgctcaatttgacaacatttctaacaaatgtgatgaatgtgaatatttgaatcggggcaaaaatggatgtatctgtcacaatgtaactcactgtcaaatttgtcatgggattcccccctgggaaaaggaaaacttgtcagattttggggattttgacgatgccaataaagaacagtaa

SEQ ID NO: 33 - DNA, 1870 bp, Artificial Sequence
  Description of Artificial Sequence:  note = synthetic construct
attctttgctctggactgctagaggaccctcgctgccatggctaccttctatgaagtcattgttcgcgtcccatttgacgtggaggaacatctgcctggaatttctgacagctttgtggactgggtaactggtcaaatttgggagctgcctccagagtcagatttaaatttgactctggttgaacagcctcagttgacggtggctgatagaattcgccgcgtgttcctgtacgagtggaacaaattttccaagcaggagtccaaattctttgtgcagtttgaaaagggatctgaatattttcatctgcacacgcttgtggagacctccggcatctcttccatggtcctcggccgctacgtgagtcagattcgcgcccagctggtgaaagtggtcttccagggaattgaaccccagatcaacgactgggtcgccatcaccaaggtaaagaagggcggagccaataaggtggtggattctgggtatattcccgcctacctgctgccgaaggtccaaccggagcttcagtgggcgtggacaaacctggacgagtataaattggccgccctgaatctggaggagcgcaaacggctcgtcgcgcagtttctggcagaatcctcgcagcgctcgcaggaggcggcttcgcagcgtgagttctcggctgacccggtcatcaaaagcaagacttcccagaaatacatggcgctcgtcaactggctcgtggagcacggcatcacttccgagaagcagtggatccaggaaaatcaggagagctacctctccttcaactccaccggcaactctcggagccagatcaaggccgcgctcgacaacgcgaccaaaattatgagtctgacaaaaagcgcggtggactacctcgtggggagctccgttcccgaggacatttcaaaaaacagaatctggcaaatttttgagatgaatggctacgacccggcctacgcgggatccatcctctacggctggtgtcagcgctccttcaacaagaggaacaccgtctggctctacggacccgccacgaccggcaagaccaacatcgcggaggccatcgcccacactgtgcccttttacggctgcgtgaactggaccaatgaaaactttccctttaatgactgtgtggacaaaatgctcatttggtgggaggagggaaagatgaccaacaaggtggttgaatccgccaaggccatcctggggggctcaaaggtgcgggtcgatcagaaatgtaaatcctctgttcaaattgattctacccctgtcattgtaacttccaatacaaacatgtgtgtggtggtggatgggaattccacgacctttgaacaccagcagccgctggaggaccgcatgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctcaattggaattcaaggtatgattgcaaatgtgactatcatgctcaatttgacaacatttctaacaaatgtgatgaatgtgaatatttgaatcggggcaaaaatggatgtatctgtcacaatgtaactcactgtcaaatttgtcatgggattcccccctgggaaaaggaaaacttgtcagattttggggattttgacgatgccaataaagaacagtaa

SEQ ID NO: 34 - protein, 330 aa, Artificial Sequence
  Description of Artificial Sequence:  note = synthetic construct
MALVNWLVEHGITSEKQWIQENQESYLSFNSTGNSRSQIKAALDNATKIMSLTKSAVDYLVGSSVPEDISKNRIWQIFEMNGYDPAYAGSILYGWCQRSFNKRNTVWLYGPATTGKTNIAEAIAHTVPFYGCVNWTNENFPFNDCVDKMLIWWEEGKMTNKVVESAKAILGGSKVRVDQKCKSSVQIDSTPVIVTSNTNMCVVVDGNSTTFEHQQPLEDRMFKFELTKRLPPDFGKITKQEVKDFFAWAKVNQVPVTHEFKVPRELAGTKGAEKSLKRPLGDVTNTSYKSLEKRARLSFVPETPRSSDVTVDPAPLRPLNWNSRLVGRSW

SEQ ID NO: 35 - DNA, 1115 bp, Artificial Sequence
  Description of Artificial Sequence:  note = synthetic construct
aggagcgcaaacggctcgtcgcgcagtttctggcagaatcctcgcagcgctcgcaggaggcggcttcgcagcgtgagttctcggctgacccggtcatcaaaagcaagacttcccagaaatacatggcgctcgtcaactggctcgtggagcacggcatcacttccgagaagcagtggatccaggaaaatcaggagagctacctctccttcaactccaccggcaactctcggagccagatcaaggccgcgctcgacaacgcgaccaaaattatgagtctgacaaaaagcgcggtggactacctcgtggggagctccgttcccgaggacatttcaaaaaacagaatctggcaaatttttgagatgaatggctacgacccggcctacgcgggatccatcctctacggctggtgtcagcgctccttcaacaagaggaacaccgtctggctctacggacccgccacgaccggcaagaccaacatcgcggaggccatcgcccacactgtgcccttttacggctgcgtgaactggaccaatgaaaactttccctttaatgactgtgtggacaaaatgctcatttggtgggaggagggaaagatgaccaacaaggtggttgaatccgccaaggccatcctggggggctcaaaggtgcgggtcgatcagaaatgtaaatcctctgttcaaattgattctacccctgtcattgtaacttccaatacaaacatgtgtgtggtggtggatgggaattccacgacctttgaacaccagcagccgctggaggaccgcatgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctcaattggaattcaagattggttggaagaagttggtga

SEQ ID NO: 36 - protein, 550 aa, Artificial Sequence
  Description of Artificial Sequence:  note = synthetic construct
MATFYEVIVRVPFDVEEHLPGISDSFVDWVTGQIWELPPESDLNLTLVEQPQLTVADRIRRVFLYEWNKFSKQESKFFVQFEKGSEYFHLHTLVETSGISSMVLGRYVSQIRAQLVKVVFQGIEPQINDWVAITKVKKGGANKVVDSGYIPAYLLPKVQPELQWAWTNLDEYKLAALNLEERKRLVAQFLAESSQRSQEAASQREFSADPVIKSKTSQKYMALVNWLVEHGITSEKQWIQENQESYLSFNSTGNSRSQIKAALDNATKIMSLTKSAVDYLVGSSVPEDISKNRIWQIFEMNGYDPAYAGSILYGWCQRSFNKRNTVWLYGPATTGKTNIAEAIAHTVPFYGCVNWTNENFPFNDCVDKMLIWWEEGKMTNKVVESAKAILGGSKVRVDQKCKSSVQIDSTPVIVTSNTNMCVVVDGNSTTFEHQQPLEDRMFKFELTKRLPPDFGKITKQEVKDFFAWAKVNQVPVTHEFKVPRELAGTKGAEKSLKRPLGDVTNTSYKSLEKRARLSFVPETPRSSDVTVDPAPLRPLNWNSRLVGRSW

SEQ ID NO: 37 - DNA, 1690 bp, Artificial Sequence
  Description of Artificial Sequence:  note = synthetic construct
attctttgctctggactgctagaggaccctcgctgccatggctaccttctatgaagtcattgttcgcgtcccatttgacgtggaggaacatctgcctggaatttctgacagctttgtggactgggtaactggtcaaatttgggagctgcctccagagtcagatttaaatttgactctggttgaacagcctcagttgacggtggctgatagaattcgccgcgtgttcctgtacgagtggaacaaattttccaagcaggagtccaaattctttgtgcagtttgaaaagggatctgaatattttcatctgcacacgcttgtggagacctccggcatctcttccatggtcctcggccgctacgtgagtcagattcgcgcccagctggtgaaagtggtcttccagggaattgaaccccagatcaacgactgggtcgccatcaccaaggtaaagaagggcggagccaataaggtggtggattctgggtatattcccgcctacctgctgccgaaggtccaaccggagcttcagtgggcgtggacaaacctggacgagtataaattggccgccctgaatctggaggagcgcaaacggctcgtcgcgcagtttctggcagaatcctcgcagcgctcgcaggaggcggcttcgcagcgtgagttctcggctgacccggtcatcaaaagcaagacttcccagaaatacatggcgctcgtcaactggctcgtggagcacggcatcacttccgagaagcagtggatccaggaaaatcaggagagctacctctccttcaactccaccggcaactctcggagccagatcaaggccgcgctcgacaacgcgaccaaaattatgagtctgacaaaaagcgcggtggactacctcgtggggagctccgttcccgaggacatttcaaaaaacagaatctggcaaatttttgagatgaatggctacgacccggcctacgcgggatccatcctctacggctggtgtcagcgctccttcaacaagaggaacaccgtctggctctacggacccgccacgaccggcaagaccaacatcgcggaggccatcgcccacactgtgcccttttacggctgcgtgaactggaccaatgaaaactttccctttaatgactgtgtggacaaaatgctcatttggtgggaggagggaaagatgaccaacaaggtggttgaatccgccaaggccatcctggggggctcaaaggtgcgggtcgatcagaaatgtaaatcctctgttcaaattgattctacccctgtcattgtaacttccaatacaaacatgtgtgtggtggtggatgggaattccacgacctttgaacaccagcagccgctggaggaccgcatgttcaaatttgaactgactaagcggctcccgccagattttggcaagattactaagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccaggctctcatttgttcccgagacgcctcgcagttcagacgtgactgttgatcccgctcctctgcgaccgctcaattggaattcaagattggttggaagaagttggtga

SEQ ID NO: 38 - DNA, 145 bp, Artificial Sequence
  Description of Artificial Sequence:  note = synthetic construct
ccatcaccaaggtaaagaagggcggagccaataaggtggtggattctgggtatattcccgcctacctgctgccgaaggtccaaccggagcttcagtgggcgtggacaaacctggacgagtataaattggccgccctgaatctgga

SEQ ID NO: 39 - DNA, 174 bp, Artificial Sequence
  Description of Artificial Sequence:  note = synthetic construct
taagcaggaagtcaaggacttttttgcttgggcaaaggtcaatcaggtgccggtgactcacgagtttaaagttcccagggaattggcgggaactaaaggggcggagaaatctctaaaacgcccactgggtgacgtcaccaatactagctataaaagtctggagaagcgggccag

SEQ ID NO: 40 - DNA, 187 bp, Artificial Sequence
  Description of Artificial Sequence:  note = synthetic construct
cactctcaagcaagggggttttgtaagcagtgatgtcataatgatgtaatgcttattgtcacgcgatagttaatgattaacagtcatgtgatgtgttttatccaataggaagaaagcgcgcgtatgagttctcgcgagacttccggggtataaaagaccgagtgaacgagcccgccgccattctttg

SEQ ID NO: 41 - DNA, 168 bp, Artificial Sequence
  Description of Artificial Sequence:  note = synthetic construct
aaacctccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacaggggggagagtgcca

SEQ ID NO: 42 - DNA, 168 bp, Artificial Sequence
  Description of Artificial Sequence:  note = synthetic construct
aaacctccttgcttgagagtgtggcactctcccccctgtcgcgttcgctcgctcgctggctcgtttgggggggcgacggccagagggccgtcgtctgccggctctttgagctgccacccccccaaacgagccagcgagcgagcgaacgcgacaggggggagagtgcca

SEQ ID NO: 43 - DNA, 8 bp, Artificial Sequence
  Description of Artificial Sequence:  note = synthetic construct
cggtgtgg

SEQ ID NO: 44 - DNA, 8 bp, Artificial Sequence
  Description of Artificial Sequence:  note = synthetic construct
cggtgtga

SEQ ID NO: 45 - DNA, 21 bp, Artificial Sequence
  Description of Artificial Sequence:  note = synthetic construct
caaaacctccttgcttgagag

SEQ ID NO: 46 - DNA, 4675 bp, Artificial Sequence
  Description of Artificial Sequence:  note = synthetic construct
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcctgtattagaggtcacgtgagtgttttgcgacattttgcgacaccatgtggtcacgctgggtatttaagcccgagtgagcacgcagggtctccattttgaagcgggaggtttgaacgcgcagccgccatgccggggttttacgagattgtgattaaggtccccagcgaccttgacgggcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgtacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctccggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccggccatggcaagccacaaggacgatgaagaaaagttttttcctcagagcggggttctcatctttgggaagcaaggctcagagaaaacaaatgtgaacattgaaaaggtcatgattacagacgaagaggaaatcggaacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacacggtcagcgtggagatcgagtgggagctgcagaaggaaaacagcaaacgctggaatcccgaaattcagtacacttccaactacaacaagtctgttaatcgtggacttaccgtggatactaatggcgtgtattcagagcctcgccccattggcaccagatacctgactcgtaatctgtaattgcttgttaatcaataaaccgtttaattcgtttcagttgaactttggtctctgcgtatttctttcttatctagtttccatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 47 - DNA, 4694 bp, Artificial Sequence
  Description of Artificial Sequence:  note = synthetic construct
gtggcactcccccccctgtcgcgttcgctcgttcgctggctcgattgggggggtggcagctcaaagagctgccagacgacggccctctgggccgtcgcccccccaatcgagccagcgaacgagcgaacgcgacaggggggggagtgccacactctctagcaagggggttttgtaggtggtgatgtcattgttgatgtcattatagttgtcacgcgatagttaatgattaacagtcatgtgatgtgtgttatccaataggatgaaagcgcgcgaatgagatctcgcgagacttccggggtataaaaggggtgagtgaacgagcccgccgccattctctgctctggactgctagaggaccctcgctgccatggctaccttctatgaagtcattgttcgcgttccatttgatgtggaagagcacctgcctggaatttctgacaactttgtagactgggtaactggtcaaatttgggagctgcctcccgagtcagatttgaatttgactctgattgagcagcctcagctgacggtggctgacagaattcgccgcgtgttcctgtacgagtggaacaaattttccaagcaggagagcaaattctttgtgcagtttgaaaagggatctgaatattttcatctgcacacgctcgtggagacctccggcatctcttctatggtccttggccgctacgtgagtcagattcgcgcccagctggtgaaggtggtgttccagaacattgagccgcggattaacgactgggtcgccatcaccaaggtaaagaagggcggagccaataaggtggtggattctgggtatattcccgcctacctgctgccgaaggtccaaccagagcttcagtgggcgtggactaacctcgaagagtataaattggccgccctcaatctggaggagcgcaaacggctcgtcgctcagtttcagcttgagtcctcgcagcgctcgcaagaggcatcttcccagagggacgtttcggctgacccggtcatcaagagcaagacttcccagaaatacatggcgctggtaagctggctggtggaacatggcatcacttccgagaagcagtggattcaggagaatcaggagagctacctgtccttcaactccacgggaaactctcggagccagattaaagccgcgcttgacaacgcgtcaaaaattatgagtctgaccaaatctgcctcagactatctcgtgggacagactgttccagaggacatttctgaaaacagaatctggcagatttttgatctcaacggctacgacccggcatacgcgggctctgttctctacggctggtgcactcgcgcctttggaaagaggaacaccgtctggctgtatggacccgcgaccaccggaaagaccaacatcgcggaagccatctctcacaccgtgcccttttatggctgtgtgaactggactaatgagaactttccctttaatgactgtgtggaaaaaatgttgatctggtgggaggagggaaagatgaccagcaaggtggtggaacccgccaaggccatcttgggggggtctagagtacgagtggatcaaaaatgtaaatcctctgtacaagtagactctaccccggtgattatcacctccaatactaacatgtgtgtggtggtggatgggaactccacgacctttgaacaccagcagccgctggaagaccgcatgttcagatttgaactcatgcggcggctcccgccagattttggcaagattaccaagcaggaagtcaaagacttttttgcttgggcaaaggtcaaccaggtgccggtgactcacgagtttatggttcccaagaaagtggcgggaactgagagggcggagacttctagaaaacgcccactggatgacgtcaccaataccaactataaaagtccggagaagcgggcccggctctcagttgttcctgagacgcctcgcagttcagacgtgcctgtagagcccgctcctctgcgacctctcaactggtcttccaggtatgaatgcagatgtgactatcatgctaaatttgactctgtaacgggggaatgtgacgagtgtgaatatttgaatcggggcaaaaatggctgtatctttcataatgctacacattgtcaaatttgtcacgctgttcctccatgggaaaaggaaaatgtgtcagattttaatgattttgatgactgtaataaagagcagtaaataaagtgagtagtcatgtcttttgttgaccaccctccagattggttggaatcgatcggcgacggctttcgtgaatttctcggccttgaggcgggtcccccgaaacccaaggccaatcaacagaagcaagataacgctcgaggtcttgtgcttcctgggtacaagtatcttggtcctgggaacggccttgataagggcgatcctgtcaattttgctgacgaggttgcccgagagcacgacctctcctaccagaaacagcttgaggcgggcgataacccttacctcaagtacaaccacgcggacgcagagtttcaggagaaactcgcttctgacacttcttttgggggaaaccttgggaaggctgttttccaggctaaaaagaggattctcgaacctcttggcctggttgagacgccggataaaacggcgcctgcggcaaaaaagaggcctctagagcagagtcctcaagagccagactcctcgagcggagttggcaagaaaggcaaacagcctgccagaaagagactcaactttgacgacgaacctggagccggagacgggcctcccccagaaggaccatcttccggagctatgtctactgagactgaaatgcgtgcagcagctggcggaaatggtggcgatgcgggacaaggtgccgagggagtgggtaatgcctccggtgattggcattgcgattccacttggtcagagagccacgtcaccaccacctcaacccgcacctgggtcctgccgacctacaacaaccacctgtacctgcggctcggctcgagcaacgccagcgacaccttcaacggattctccaccccctggggatactttgactttaaccgcttccactgccacttctcgccaagagactggcaaaggctcatcaacaaccactggggactgcgccccaaaagcatgcaagtccgcatcttcaacatccaagttaaggaggtcacgacgtctaacggggagacgaccgtatccaacaacctcaccagcacggtccagatctttgcggacagcacgtacgagctcccgtacgtgatggatgcaggtcaggagggcagcttgcctcctttccccaacgacgtgttcatggtgcctcagtacgggtactgcggactggtaaccggaggcagctctcaaaaccagacagacagaaatgccttctactgtctggagtactttcccagccagatgctgagaaccggaaacaactttgagatggtgtacaagtttgaaaacgtgcccttccactccatgtacgctcacagccagagcctggataggctgatgaacccgctgctggaccagtacctgtgggagctccagtctaccacctctggaggaactctcaaccagggcaattcagccaccaactttgccaagctgaccaaaacaaacttttctggctaccgcaaaaactggctcccggggcccatgatgaagcagcagagattctccaagactgccagtcaaaactacaagattccccagggaagaaacaacagtctgctccattatgagaccagaactaccctcgacggaagatggagcaattttgccccgggaacggccatggcaaccgcagccaacgacgccaccgacttctctcaggcccagctcatctttgcggggcccaacatcaccggcaacaccaccacagatgccaataacctgatgttcacttcagaagatgaacttagggccaccaacccccgggacactgacctgtttggccacctggcaaccaaccagcaaaacgccaccaccgttcctaccgtagacgacgtggacggagtcggcgtgtacccgggaatggtgtggcaggacagagacatttactaccaagggcccatttgggccaaaattccacacacggatggacactttcacccgtctcctctcattggcggatttggactgaaaagcccgcctccacaaatattcatcaaaaacactcctgtacccgccaatcccgcaacgaccttctctccggccagaatcaacagcttcatcacccagtacagcaccggacaggtggctgtcaaaatagaatgggaaatccagaaggagcggtccaagagatggaacccagaggtccagttcacgtccaactacggagcacaggactcgcttctctgggctcccgacaacgccggagcctacaaagagcccagggccattggatcccgatacctcaccaaccacctctagcccaattctgttgcataccctcaataaaccgtgtattcgtttcagtaaaatactgcctcttgtggtcattcggcgtacaacagcttacaacaacaacaaaacccccttgctagagagtgtggcactcccccccctgtcgcgttcgctcgttcgctggctcgattgggggggtggcagctcaaagagctgccagacgacggccctctgggccgtcgcccccccaatcgagccagcgaacgagcgaacgcgacaggggggggagtgccac

SEQ ID NO: 48 - DNA, 1833 bp, Artificial Sequence
  Description of Artificial Sequence:  note = synthetic construct
atggctaccttctatgaagtcattgttcgcgttccatttgatgtggaagagcacctgcctggaatttctgacaactttgtagactgggtaactggtcaaatttgggagctgcctcccgagtcagatttgaatttgactctgattgagcagcctcagctgacggtggctgacagaattcgccgcgtgttcctgtacgagtggaacaaattttccaagcaggagagcaaattctttgtgcagtttgaaaagggatctgaatattttcatctgcacacgctcgtggagacctccggcatctcttctatggtccttggccgctacgtgagtcagattcgcgcccagctggtgaaggtggtgttccagaacattgagccgcggattaacgactgggtcgccatcaccaaggtaaagaagggcggagccaataaggtggtggattctgggtatattcccgcctacctgctgccgaaggtccaaccagagcttcagtgggcgtggactaacctcgaagagtataaattggccgccctcaatctggaggagcgcaaacggctcgtcgctcagtttcagcttgagtcctcgcagcgctcgcaagaggcatcttcccagagggacgtttcggctgacccggtcatcaagagcaagacttcccagaaatacatggcgctggtaagctggctggtggaacatggcatcacttccgagaagcagtggattcaggagaatcaggagagctacctgtccttcaactccacgggaaactctcggagccagattaaagccgcgcttgacaacgcgtcaaaaattatgagtctgaccaaatctgcctcagactatctcgtgggacagactgttccagaggacatttctgaaaacagaatctggcagatttttgatctcaacggctacgacccggcatacgcgggctctgttctctacggctggtgcactcgcgcctttggaaagaggaacaccgtctggctgtatggacccgcgaccaccggaaagaccaacatcgcggaagccatctctcacaccgtgcccttttatggctgtgtgaactggactaatgagaactttccctttaatgactgtgtggaaaaaatgttgatctggtgggaggagggaaagatgaccagcaaggtggtggaacccgccaaggccatcttgggggggtctagagtacgagtggatcaaaaatgtaaatcctctgtacaagtagactctaccccggtgattatcacctccaatactaacatgtgtgtggtggtggatgggaactccacgacctttgaacaccagcagccgctggaagaccgcatgttcagatttgaactcatgcggcggctcccgccagattttggcaagattaccaagcaggaagtcaaagacttttttgcttgggcaaaggtcaaccaggtgccggtgactcacgagtttatggttcccaagaaagtggcgggaactgagagggcggagacttctagaaaacgcccactggatgacgtcaccaataccaactataaaagtccggagaagcgggcccggctctcagttgttcctgagacgcctcgcagttcagacgtgcctgtagagcccgctcctctgcgacctctcaactggtcttccaggtatgaatgcagatgtgactatcatgctaaatttgactctgtaacgggggaatgtgacgagtgtgaatatttgaatcggggcaaaaatggctgtatctttcataatgctacacattgtcaaatttgtcacgctgttcctccatgggaaaaggaaaatgtgtcagattttaatgattttgatgactgtaataaagagcagtaa

SEQ ID NO: 49 - protein, 610 aa, Artificial Sequence
  Description of Artificial Sequence:  note = synthetic construct
MATFYEVIVRVPFDVEEHLPGISDNFVDWVTGQIWELPPESDLNLTLIEQPQLTVADRIRRVFLYEWNKFSKQESKFFVQFEKGSEYFHLHTLVETSGISSMVLGRYVSQIRAQLVKVVFQNIEPRINDWVAITKVKKGGANKVVDSGYIPAYLLPKVQPELQWAWTNLEEYKLAALNLEERKRLVAQFQLESSQRSQEASSQRDVSADPVIKSKTSQKYMALVSWLVEHGITSEKQWIQENQESYLSFNSTGNSRSQIKAALDNASKIMSLTKSASDYLVGQTVPEDISENRIWQIFDLNGYDPAYAGSVLYGWCTRAFGKRNTVWLYGPATTGKTNIAEAISHTVPFYGCVNWTNENFPFNDCVEKMLIWWEEGKMTSKVVEPAKAILGGSRVRVDQKCKSSVQVDSTPVIITSNTNMCVVVDGNSTTFEHQQPLEDRMFRFELMRRLPPDFGKITKQEVKDFFAWAKVNQVPVTHEFMVPKKVAGTERAETSRKRPLDDVTNTNYKSPEKRARLSVVPETPRSSDVPVEPAPLRPLNWSSRYECRCDYHAKFDSVTGECDECEYLNRGKNGCIFHNATHCQICHAVPPWEKENVSDFNDFDDCNKEQ

SEQ ID NO: 50 - DNA, 1173 bp, Artificial Sequence
  Description of Artificial Sequence:  note = synthetic construct
atggcgctggtaagctggctggtggaacatggcatcacttccgagaagcagtggattcaggagaatcaggagagctacctgtccttcaactccacgggaaactctcggagccagattaaagccgcgcttgacaacgcgtcaaaaattatgagtctgaccaaatctgcctcagactatctcgtgggacagactgttccagaggacatttctgaaaacagaatctggcagatttttgatctcaacggctacgacccggcatacgcgggctctgttctctacggctggtgcactcgcgcctttggaaagaggaacaccgtctggctgtatggacccgcgaccaccggaaagaccaacatcgcggaagccatctctcacaccgtgcccttttatggctgtgtgaactggactaatgagaactttccctttaatgactgtgtggaaaaaatgttgatctggtgggaggagggaaagatgaccagcaaggtggtggaacccgccaaggccatcttgggggggtctagagtacgagtggatcaaaaatgtaaatcctctgtacaagtagactctaccccggtgattatcacctccaatactaacatgtgtgtggtggtggatgggaactccacgacctttgaacaccagcagccgctggaagaccgcatgttcagatttgaactcatgcggcggctcccgccagattttggcaagattaccaagcaggaagtcaaagacttttttgcttgggcaaaggtcaaccaggtgccggtgactcacgagtttatggttcccaagaaagtggcgggaactgagagggcggagacttctagaaaacgcccactggatgacgtcaccaataccaactataaaagtccggagaagcgggcccggctctcagttgttcctgagacgcctcgcagttcagacgtgcctgtagagcccgctcctctgcgacctctcaactggtcttccaggtatgaatgcagatgtgactatcatgctaaatttgactctgtaacgggggaatgtgacgagtgtgaatatttgaatcggggcaaaaatggctgtatctttcataatgctacacattgtcaaatttgtcacgctgttcctccatgggaaaaggaaaatgtgtcagattttaatgattttgatgactgtaataaagagcagtaa

SEQ ID NO: 51 - protein, 390 aa, Artificial Sequence
  Description of Artificial Sequence:  note = synthetic construct
MALVSWLVEHGITSEKQWIQENQESYLSFNSTGNSRSQIKAALDNASKIMSLTKSASDYLVGQTVPEDISENRIWQIFDLNGYDPAYAGSVLYGWCTRAFGKRNTVWLYGPATTGKTNIAEAISHTVPFYGCVNWTNENFPFNDCVEKMLIWWEEGKMTSKVVEPAKAILGGSRVRVDQKCKSSVQVDSTPVIITSNTNMCVVVDGNSTTFEHQQPLEDRMFRFELMRRLPPDFGKITKQEVKDFFAWAKVNQVPVTHEFMVPKKVAGTERAETSRKRPLDDVTNTNYKSPEKRARLSVVPETPRSSDVPVEPAPLRPLNWSSRYECRCDYHAKFDSVTGECDECEYLNRGKNGCIFHNATHCQICHAVPPWEKENVSDFNDFDDCNKEQ

SEQ ID NO: 52 - DNA, 2211 bp, Artificial Sequence
  Description of Artificial Sequence:  note = synthetic construct
atgtcttttgttgaccaccctccagattggttggaatcgatcggcgacggctttcgtgaatttctcggccttgaggcgggtcccccgaaacccaaggccaatcaacagaagcaagataacgctcgaggtcttgtgcttcctgggtacaagtatcttggtcctgggaacggccttgataagggcgatcctgtcaattttgctgacgaggttgcccgagagcacgacctctcctaccagaaacagcttgaggcgggcgataacccttacctcaagtacaaccacgcggacgcagagtttcaggagaaactcgcttctgacacttcttttgggggaaaccttgggaaggctgttttccaggctaaaaagaggattctcgaacctcttggcctggttgagacgccggataaaacggcgcctgcggcaaaaaagaggcctctagagcagagtcctcaagagccagactcctcgagcggagttggcaagaaaggcaaacagcctgccagaaagagactcaactttgacgacgaacctggagccggagacgggcctcccccagaaggaccatcttccggagctatgtctactgagactgaaatgcgtgcagcagctggcggaaatggtggcgatgcgggacaaggtgccgagggagtgggtaatgcctccggtgattggcattgcgattccacttggtcagagagccacgtcaccaccacctcaacccgcacctgggtcctgccgacctacaacaaccacctgtacctgcggctcggctcgagcaacgccagcgacaccttcaacggattctccaccccctggggatactttgactttaaccgcttccactgccacttctcgccaagagactggcaaaggctcatcaacaaccactggggactgcgccccaaaagcatgcaagtccgcatcttcaacatccaagttaaggaggtcacgacgtctaacggggagacgaccgtatccaacaacctcaccagcacggtccagatctttgcggacagcacgtacgagctcccgtacgtgatggatgcaggtcaggagggcagcttgcctcctttccccaacgacgtgttcatggtgcctcagtacgggtactgcggactggtaaccggaggcagctctcaaaaccagacagacagaaatgccttctactgtctggagtactttcccagccagatgctgagaaccggaaacaactttgagatggtgtacaagtttgaaaacgtgcccttccactccatgtacgctcacagccagagcctggataggctgatgaacccgctgctggaccagtacctgtgggagctccagtctaccacctctggaggaactctcaaccagggcaattcagccaccaactttgccaagctgaccaaaacaaacttttctggctaccgcaaaaactggctcccggggcccatgatgaagcagcagagattctccaagactgccagtcaaaactacaagattccccagggaagaaacaacagtctgctccattatgagaccagaactaccctcgacggaagatggagcaattttgccccgggaacggccatggcaaccgcagccaacgacgccaccgacttctctcaggcccagctcatctttgcggggcccaacatcaccggcaacaccaccacagatgccaataacctgatgttcacttcagaagatgaacttagggccaccaacccccgggacactgacctgtttggccacctggcaaccaaccagcaaaacgccaccaccgttcctaccgtagacgacgtggacggagtcggcgtgtacccgggaatggtgtggcaggacagagacatttactaccaagggcccatttgggccaaaattccacacacggatggacactttcacccgtctcctctcattggcggatttggactgaaaagcccgcctccacaaatattcatcaaaaacactcctgtacccgccaatcccgcaacgaccttctctccggccagaatcaacagcttcatcacccagtacagcaccggacaggtggctgtcaaaatagaatgggaaatccagaaggagcggtccaagagatggaacccagaggtccagttcacgtccaactacggagcacaggactcgcttctctgggctcccgacaacgccggagcctacaaagagcccagggccattggatcccgatacctcaccaaccacctctag

SEQ ID NO: 53 - protein, 736 aa, Artificial Sequence
  Description of Artificial Sequence:  note = synthetic construct
MSFVDHPPDWLESIGDGFREFLGLEAGPPKPKANQQKQDNARGLVLPGYKYLGPGNGLDKGDPVNFADEVAREHDLSYQKQLEAGDNPYLKYNHADAEFQEKLASDTSFGGNLGKAVFQAKKRILEPLGLVETPDKTAPAAKKRPLEQSPQEPDSSSGVGKKGKQPARKRLNFDDEPGAGDGPPPEGPSSGAMSTETEMRAAAGGNGGDAGQGAEGVGNASGDWHCDSTWSESHVTTTSTRTWVLPTYNNHLYLRLGSSNASDTFNGFSTPWGYFDFNRFHCHFSPRDWQRLINNHWGLRPKSMQVRIFNIQVKEVTTSNGETTVSNNLTSTVQIFADSTYELPYVMDAGQEGSLPPFPNDVFMVPQYGYCGLVTGGSSQNQTDRNAFYCLEYFPSQMLRTGNNFEMVYKFENVPFHSMYAHSQSLDRLMNPLLDQYLWELQSTTSGGTLNQGNSATNFAKLTKTNFSGYRKNWLPGPMMKQQRFSKTASQNYKIPQGRNNSLLHYETRTTLDGRWSNFAPGTAMATAANDATDFSQAQLIFAGPNITGNTTTDANNLMFTSEDELRATNPRDTDLFGHLATNQQNATTVPTVDDVDGVGVYPGMVWQDRDIYYQGPIWAKIPHTDGHFHPSPLIGGFGLKSPPPQIFIKNTPVPANPATTFSPARINSFITQYSTGQVAVKIEWEIQKERSKRWNPEVQFTSNYGAQDSLLWAPDNAGAYKEPRAIGSRYLTNHL

SEQ ID NO: 54 - DNA, 1803 bp, Artificial Sequence
  Description of Artificial Sequence:  note = synthetic construct
acggcgcctgcggcaaaaaagaggcctctagagcagagtcctcaagagccagactcctcgagcggagttggcaagaaaggcaaacagcctgccagaaagagactcaactttgacgacgaacctggagccggagacgggcctcccccagaaggaccatcttccggagctatgtctactgagactgaaatgcgtgcagcagctggcggaaatggtggcgatgcgggacaaggtgccgagggagtgggtaatgcctccggtgattggcattgcgattccacttggtcagagagccacgtcaccaccacctcaacccgcacctgggtcctgccgacctacaacaaccacctgtacctgcggctcggctcgagcaacgccagcgacaccttcaacggattctccaccccctggggatactttgactttaaccgcttccactgccacttctcgccaagagactggcaaaggctcatcaacaaccactggggactgcgccccaaaagcatgcaagtccgcatcttcaacatccaagttaaggaggtcacgacgtctaacggggagacgaccgtatccaacaacctcaccagcacggtccagatctttgcggacagcacgtacgagctcccgtacgtgatggatgcaggtcaggagggcagcttgcctcctttccccaacgacgtgttcatggtgcctcagtacgggtactgcggactggtaaccggaggcagctctcaaaaccagacagacagaaatgccttctactgtctggagtactttcccagccagatgctgagaaccggaaacaactttgagatggtgtacaagtttgaaaacgtgcccttccactccatgtacgctcacagccagagcctggataggctgatgaacccgctgctggaccagtacctgtgggagctccagtctaccacctctggaggaactctcaaccagggcaattcagccaccaactttgccaagctgaccaaaacaaacttttctggctaccgcaaaaactggctcccggggcccatgatgaagcagcagagattctccaagactgccagtcaaaactacaagattccccagggaagaaacaacagtctgctccattatgagaccagaactaccctcgacggaagatggagcaattttgccccgggaacggccatggcaaccgcagccaacgacgccaccgacttctctcaggcccagctcatctttgcggggcccaacatcaccggcaacaccaccacagatgccaataacctgatgttcacttcagaagatgaacttagggccaccaacccccgggacactgacctgtttggccacctggcaaccaaccagcaaaacgccaccaccgttcctaccgtagacgacgtggacggagtcggcgtgtacccgggaatggtgtggcaggacagagacatttactaccaagggcccatttgggccaaaattccacacacggatggacactttcacccgtctcctctcattggcggatttggactgaaaagcccgcctccacaaatattcatcaaaaacactcctgtacccgccaatcccgcaacgaccttctctccggccagaatcaacagcttcatcacccagtacagcaccggacaggtggctgtcaaaatagaatgggaaatccagaaggagcggtccaagagatggaacccagaggtccagttcacgtccaactacggagcacaggactcgcttctctgggctcccgacaacgccggagcctacaaagagcccagggccattggatcccgatacctcaccaaccacctctag

SEQ ID NO: 55 - protein, 600 aa, Artificial Sequence
  Description of Artificial Sequence:  note = synthetic construct
TAPAAKKRPLEQSPQEPDSSSGVGKKGKQPARKRLNFDDEPGAGDGPPPEGPSSGAMSTETEMRAAAGGNGGDAGQGAEGVGNASGDWHCDSTWSESHVTTTSTRTWVLPTYNNHLYLRLGSSNASDTFNGFSTPWGYFDFNRFHCHFSPRDWQRLINNHWGLRPKSMQVRIFNIQVKEVTTSNGETTVSNNLTSTVQIFADSTYELPYVMDAGQEGSLPPFPNDVFMVPQYGYCGLVTGGSSQNQTDRNAFYCLEYFPSQMLRTGNNFEMVYKFENVPFHSMYAHSQSLDRLMNPLLDQYLWELQSTTSGGTLNQGNSATNFAKLTKTNFSGYRKNWLPGPMMKQQRFSKTASQNYKIPQGRNNSLLHYETRTTLDGRWSNFAPGTAMATAANDATDFSQAQLIFAGPNITGNTTTDANNLMFTSEDELRATNPRDTDLFGHLATNQQNATTVPTVDDVDGVGVYPGMVWQDRDIYYQGPIWAKIPHTDGHFHPSPLIGGFGLKSPPPQIFIKNTPVPANPATTFSPARINSFITQYSTGQVAVKIEWEIQKERSKRWNPEVQFTSNYGAQDSLLWAPDNAGAYKEPRAIGSRYLTNHL

SEQ ID NO: 56 - DNA, 1617 bp, Artificial Sequence
  Description of Artificial Sequence:  note = synthetic construct
atgcgtgcagcagctggcggaaatggtggcgatgcgggacaaggtgccgagggagtgggtaatgcctccggtgattggcattgcgattccacttggtcagagagccacgtcaccaccacctcaacccgcacctgggtcctgccgacctacaacaaccacctgtacctgcggctcggctcgagcaacgccagcgacaccttcaacggattctccaccccctggggatactttgactttaaccgcttccactgccacttctcgccaagagactggcaaaggctcatcaacaaccactggggactgcgccccaaaagcatgcaagtccgcatcttcaacatccaagttaaggaggtcacgacgtctaacggggagacgaccgtatccaacaacctcaccagcacggtccagatctttgcggacagcacgtacgagctcccgtacgtgatggatgcaggtcaggagggcagcttgcctcctttccccaacgacgtgttcatggtgcctcagtacgggtactgcggactggtaaccggaggcagctctcaaaaccagacagacagaaatgccttctactgtctggagtactttcccagccagatgctgagaaccggaaacaactttgagatggtgtacaagtttgaaaacgtgcccttccactccatgtacgctcacagccagagcctggataggctgatgaacccgctgctggaccagtacctgtgggagctccagtctaccacctctggaggaactctcaaccagggcaattcagccaccaactttgccaagctgaccaaaacaaacttttctggctaccgcaaaaactggctcccggggcccatgatgaagcagcagagattctccaagactgccagtcaaaactacaagattccccagggaagaaacaacagtctgctccattatgagaccagaactaccctcgacggaagatggagcaattttgccccgggaacggccatggcaaccgcagccaacgacgccaccgacttctctcaggcccagctcatctttgcggggcccaacatcaccggcaacaccaccacagatgccaataacctgatgttcacttcagaagatgaacttagggccaccaacccccgggacactgacctgtttggccacctggcaaccaaccagcaaaacgccaccaccgttcctaccgtagacgacgtggacggagtcggcgtgtacccgggaatggtgtggcaggacagagacatttactaccaagggcccatttgggccaaaattccacacacggatggacactttcacccgtctcctctcattggcggatttggactgaaaagcccgcctccacaaatattcatcaaaaacactcctgtacccgccaatcccgcaacgaccttctctccggccagaatcaacagcttcatcacccagtacagcaccggacaggtggctgtcaaaatagaatgggaaatccagaaggagcggtccaagagatggaacccagaggtccagttcacgtccaactacggagcacaggactcgcttctctgggctcccgacaacgccggagcctacaaagagcccagggccattggatcccgatacctcaccaaccacctctag

SEQ ID NO: 57 - protein, 538 aa, Artificial Sequence
  Description of Artificial Sequence:  note = synthetic construct
MRAAAGGNGGDAGQGAEGVGNASGDWHCDSTWSESHVTTTSTRTWVLPTYNNHLYLRLGSSNASDTFNGFSTPWGYFDFNRFHCHFSPRDWQRLINNHWGLRPKSMQVRIFNIQVKEVTTSNGETTVSNNLTSTVQIFADSTYELPYVMDAGQEGSLPPFPNDVFMVPQYGYCGLVTGGSSQNQTDRNAFYCLEYFPSQMLRTGNNFEMVYKFENVPFHSMYAHSQSLDRLMNPLLDQYLWELQSTTSGGTLNQGNSATNFAKLTKTNFSGYRKNWLPGPMMKQQRFSKTASQNYKIPQGRNNSLLHYETRTTLDGRWSNFAPGTAMATAANDATDFSQAQLIFAGPNITGNTTTDANNLMFTSEDELRATNPRDTDLFGHLATNQQNATTVPTVDDVDGVGVYPGMVWQDRDIYYQGPIWAKIPHTDGHFHPSPLIGGFGLKSPPPQIFIKNTPVPANPATTFSPARINSFITQYSTGQVAVKIEWEIQKERSKRWNPEVQFTSNYGAQDSLLWAPDNAGAYKEPRAIGSRYLTNHL

SEQ ID NO: 58 - DNA, 150 bp, Artificial Sequence
  Description of Artificial Sequence:  note = synthetic construct
gtggcactcccccccctgtcgcgttcgctcgttcgctggctcgattgggggggtggcagctcaaagagctgccagacgacggccctctgggccgtcgcccccccaatcgagccagcgaacgagcgaacgcgacaggggggggagtgccac

SEQ ID NO: 59 - DNA, 20 bp, Artificial Sequence
  Description of Artificial Sequence:  note = synthetic construct
ctctagcaagggggttttgt

SEQ ID NO: 60 - DNA, 7 bp, Artificial Sequence
  Description of Artificial Sequence:  note = synthetic construct
agtgtgg

SEQ ID NO: 61 - DNA, 158 bp, Artificial Sequence
  Description of Artificial Sequence:  note = synthetic construct
aggtggtgatgtcattgttgatgtcattatagttgtcacgcgatagttaatgattaacagtcatgtgatgtgtgttatccaataggatgaaagcgcgcgaatgagatctcgcgagacttccggggtataaaaggggtgagtgaacgagcccgccgcca

SEQ ID NO: 62 - DNA, 112 bp, Artificial Sequence
  Description of Artificial Sequence:  note = synthetic construct
ggtggattctgggtatattcccgcctacctgctgccgaaggtccaaccagagcttcagtgggcgtggactaacctcgaagagtataaattggccgccctcaatctggaggag

SEQ ID NO: 63 - DNA, 169 bp, Artificial Sequence
  Description of Artificial Sequence:  note = synthetic construct
agtcaaagacttttttgcttgggcaaaggtcaaccaggtgccggtgactcacgagtttatggttcccaagaaagtggcgggaactgagagggcggagacttctagaaaacgcccactggatgacgtcaccaataccaactataaaagtccggagaagcgggcccggctc

SEQ ID NO: 64 - DNA, 4721 bp, Artificial Sequence
  Description of Artificial Sequence:  note = synthetic construct
ttggccactccctctatgcgcgctcgctcgctcggtggggcctgcggaccaaaggtccgcagacggcagagctctgctctgccggccccaccgagcgagcgagcgcgcatagagggagtggccaactccatcactaggggtaccgcgaagcgcctcccacgctgccgcgtcagcgctgacgtaaatcacgtcataggggagtggtcctgtattagctgtcacgtgagtgcttttgcgacattttgcgacaccacgtggccatttgaggtatatatggccgagtgagcgagcaggatctccattttgaccgcgaaatttgaacgagcagcagccatgccgggtttctacgagatcgtgatcaaggtgccgagcgacctggacgagcacctgccgggcatttctgactcgtttgtgaactgggtggccgagaaggaatgggagctgcccccggattctgacatggatctgaatctgatcgagcaggcacccctgaccgtggccgagaagctgcagcgcgacttcctggtccaatggcgccgcgtgagtaaggccccggaggccctgttctttgttcagttcgagaagggcgagagctacttccaccttcacgttctggtggagaccacgggggtcaagtccatggtgctaggccgcttcctgagtcagattcgggagaagctggtccagaccatctaccgcggggtcgagcccacgctgcccaactggttcgcggtgaccaagacgcgtaatggcgccggcggggggaacaaggtggtggacgagtgctacatccccaactacctcctgcccaagacccagcccgagctgcagtgggcgtggactaacatggaggagtatataagcgcgtgtttgaacctggccgaacgcaaacggctcgtggcgcagcacctgacccacgtcagccagacgcaggagcagaacaaggagaatctgaaccccaattctgacgcgcccgtgatcaggtcaaaaacctccgcgcgctacatggagctggtcgggtggctggtggaccggggcatcacctccgagaagcagtggatccaggaggaccaggcctcgtacatctccttcaacgccgcctccaactcgcggtcccagatcaaggccgcgctggacaatgccggcaagatcatggcgctgaccaaatccgcgcccgactacctggtggggccctcgctgcccgcggacattaaaaccaaccgcatctaccgcatcctggagctgaacgggtacgatcctgcctacgccggctccgtctttctcggctgggcccagaaaaagttcgggaagcgcaacaccatctggctgtttgggcccgccaccaccggcaagaccaacattgcggaagccatcgcccacgccgtgcccttctacggctgcgtcaactggaccaatgagaactttcccttcaacgattgcgtcgacaagatggtgatctggtgggaggagggcaagatgacggccaaggtcgtggagtccgccaaggccattctcggcggcagcaaggtgcgcgtggaccaaaagtgcaagtcgtccgcccagatcgaccccacccccgtgatcgtcacctccaacaccaacatgtgcgccgtgattgacgggaacagcaccaccttcgagcaccagcagccgttgcaggaccggatgttcaaatttgaactcacccgccgtctggagcacgactttggcaaggtgacgaagcaggaagtcaaagagttcttccgctgggccagtgatcacgtgaccgaggtggcgcatgagttctacgtcagaaagggcggagccagcaaaagacccgcccccgatgacgcggatataagcgagcccaagcgggcctgcccctcagtcgcggatccatcgacgtcagacgcggaaggagctccggtggactttgccgacaggtaccaaaacaaatgttctcgtcacgcgggcatgattcagatgctgtttccctgcaaaacgtgcgagagaatgaatcagaatttcaacatttgcttcacacacggggtcagagactgtttagagtgtttccccggcgtgtcagaatctcaaccggtcgtcagaaaaaagacgtatcggaaactctgcgcgattcatcatctgctggggcgggcgcccgagattgcttgctcggcctgcgacctggtcaacgtggacctggacgactgcgtttctgagcaataaatgacttaaaccaggtatggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctgtaattgcatgttaatcaataaaccggttgattcgtttcagttgaactttggtctcctgtgcttcttatcttatcggtttccatagcaactggttacacattaactgcttgggtgcgcttcacgataagaacactgacgtcaccgcggtacccctagtgatggagttggccactccctctatgcgcgctcgctcgctcggtggggcctgcggaccaaaggtccgcagacggcagagctctgctctgccggccccaccgagcgagcgagcgcgcatagagggagtggccaa

SEQ ID NO: 65 - protein, 623 aa, Artificial Sequence
  Description of Artificial Sequence:  note = synthetic construct
MPGFYEIVIKVPSDLDEHLPGISDSFVNWVAEKEWELPPDSDMDLNLIEQAPLTVAEKLQRDFLVQWRRVSKAPEALFFVQFEKGESYFHLHVLVETTGVKSMVLGRFLSQIREKLVQTIYRGVEPTLPNWFAVTKTRNGAGGGNKVVDECYIPNYLLPKTQPELQWAWTNMEEYISACLNLAERKRLVAQHLTHVSQTQEQNKENLNPNSDAPVIRSKTSARYMELVGWLVDRGITSEKQWIQEDQASYISFNAASNSRSQIKAALDNAGKIMALTKSAPDYLVGPSLPADIKTNRIYRILELNGYDPAYAGSVFLGWAQKKFGKRNTIWLFGPATTGKTNIAEAIAHAVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLEHDFGKVTKQEVKEFFRWASDHVTEVAHEFYVRKGGASKRPAPDDADISEPKRACPSVADPSTSDAEGAPVDFADRYQNKCSRHAGMIQMLFPCKTCERMNQNFNICFTHGVRDCLECFPGVSESQPVVRKKTYRKLCAIHHLLGRAPEIACSACDLVNVDLDDCVSEQ

SEQ ID NO: 66 - protein, 737 aa, Artificial Sequence
  Description of Artificial Sequence:  note = synthetic construct
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 67 - protein, 8 aa, Artificial Sequence
  Description of Artificial Sequence:  note = synthetic construct
GSSNASDT

SEQ ID NO: 68 - protein, 14 aa, Artificial Sequence
  Description of Artificial Sequence:  note = synthetic construct
TTSGGTLNQGNSAT

SEQ ID NO: 69 - protein, 6 aa, Artificial Sequence
  Description of Artificial Sequence:  note = synthetic construct
NGRAHA

SEQ ID NO: 70 - protein, 7 aa, Artificial Sequence
  Description of Artificial Sequence:  note = synthetic construct
SIGYPLP

SEQ ID NO: 71 - protein, 10 aa, Artificial Sequence
  Description of Artificial Sequence:  note = synthetic construct
KFNKPFVFLI

SEQ ID NO: 72 - protein, 22 aa, Artificial Sequence
  Description of Artificial Sequence:  note = synthetic construct
NISLDNPLENPSSLFDLVARIK